SEQUENCE LISTING (38 sequences)

SEQ ID NO: 1 - DNA, 804 bp, unidentified
  misc_feature 1..804: note = C15EN2
  source 1..804: mol_type = other DNA
atggcggattatgcgaaaggcccggcaccgaccgttgcgctgctgacagcaccggcaggtccgtatcagaccgcaacctataaaatcgcagcagcagatgcaaaagcctttaactatggtggtgcaaccgttcattatccggttgcaaaagcaccgggtgaaacctttggtgttgttgttatggttccgggttttctggcatttcaggcagtttatgattggctggttagccgtgttgcaagccatggttttgttgttgtgaatatggataccgttggtcgtggtgattatccggatgaacgtgcagaacaggcagccggtgcactgcgtcatgttgttagcctggcacaggcaggcaccgcaccgtatgcaccggttgcagataccagccgtcgtgcagttatgggtaatagcatgggtggtggtgccagcctgagcgcagcagttgttgatccgacactgaaagccgttgttgccctgcagccgtggcataccaccaaaagttttaccggtgttgcgagcccgacactggttgttgcatgtgaaaaagatagcattgcaccgaacaaaagccattccgatgtgttttataacagcctgtctgcagcactgcctcgtgcagaaattgaaatgcgtggtgcaggtcatctgtgtgcaacctttctggcaaccgcagcacagaaaaccaccatggcaaaaagcagcgttgcatggctgaaacgttttctggataatgatatgcgttatgacgccatggttaaaggtggtattgatcagggtgaatttagccgttttgtggtgtaa

SEQ ID NO: 2 - protein, 267 aa, unidentified
  REGION 1..267: note = C15EN2
MADYAKGPAPTVALLTAPAGPYQTATYKIAAADAKAFNYGGATVHYPVAKAPGETFGVVVMVPGFLAFQAVYDWLVSRVASHGFVVVNMDTVGRGDYPDERAEQAAGALRHVVSLAQAGTAPYAPVADTSRRAVMGNSMGGGASLSAAVVDPTLKAVVALQPWHTTKSFTGVASPTLVVACEKDSIAPNKSHSDVFYNSLSAALPRAEIEMRGAGHLCATFLATAAQKTTMAKSSVAWLKRFLDNDMRYDAMVKGGIDQGEFSRFVV

SEQ ID NO: 3 - DNA, 969 bp, unidentified
  misc_feature 1..969: note = RR1102
  source 1..969: mol_type = other DNA
atgagtgaaaatatgaatcggccattagattattctcagaaggccaattggtatcagattccagaagttacaaaggaattcgacacattttatgtatatgcaaccgagtacatcctgagtagcatggaggagggtgctccggattatgctgatatggaaaatgccgaaatgctggagggtgctgctgcagaatatatgttgcacgccaccgcatacgcagattccaccaacgtgttcatgccttactaccgtcaagtaggcttgcgttatgccggggtcgtctggaagagggacggaatcttcgatgcctccgttgctggtatgccttatggcgacattgttgccgcattagactattacttcgagcactacaacaacggccgcccattcatcatcgctggccatagtcagggatcggctattattaagatggtgctaaaaaaatattttgtggagcaccccgattactacaagcgtatgattgccgcttacccgattggctacgctttcacgaaggacgagttcaagacatacccacatatgaagtttgccactggtgagtgcgatacgggtgttatcatcacctggaataccgaaggacctaagaacagggaagtaaacgctgatacgtgtgtgctgcagccgggttctatgagtataaacccgcttaactggaaactggacgacacttatgctccggccagcatgaatctggggtctctttttccgaacaaagataccggcaaactggagattcaggacctgggtgcggacgcacaggtgttccctgaccgcggagtggtagttacccatgccatgggtgaggaaatgaccgaggagaccgccaaagtggcagccgaattcttcggccctgatggccgtcacggcgaagattacgtacttttctattgcaacattaaggataatgtggctaaacgtatctcggcctatatggcaaaccgcaagtag

SEQ ID NO: 4 - protein, 322 aa, unidentified
  REGION 1..322: note = RR1102
MSENMNRPLDYSQKANWYQIPEVTKEFDTFYVYATEYILSSMEEGAPDYADMENAEMLEGAAAEYMLHATAYADSTNVFMPYYRQVGLRYAGVVWKRDGIFDASVAGMPYGDIVAALDYYFEHYNNGRPFIIAGHSQGSAIIKMVLKKYFVEHPDYYKRMIAAYPIGYAFTKDEFKTYPHMKFATGECDTGVIITWNTEGPKNREVNADTCVLQPGSMSINPLNWKLDDTYAPASMNLGSLFPNKDTGKLEIQDLGADAQVFPDRGVVVTHAMGEEMTEETAKVAAEFFGPDGRHGEDYVLFYCNIKDNVAKRISAYMANRK

SEQ ID NO: 5 - DNA, 963 bp, unidentified
  misc_feature 1..963: note = N20O1
  source 1..963: mol_type = other DNA
atggaaacaactaccgattactcccagaaggctaactggtgtaaattcccccaaattaccaaggacgtagatacgttctacatatttgctactgagtatatcatgggaagctttgaagaaggtgccccagattatggtacactcgacaatcaagagatgatagagggtatgaatgttgagtatttagtacatgcaacaacatatgccgactcaaccaatgtgttcgctccatactaccgccagactggtctaaggtttgctggtgacatatataagagagatggcaatttcgaggctgcgcttatcggtacgcccattgatgacatcatggcagccctcgactactatttcgagcactacaacgagggccgtccgttcatcatcgcaggtcacagccaaggctccgcattggtcaaggtggtattgatgaaatacttcaaggaacacccagactattaccggcgcatggtggctgcctatgttataggttactccgtcacaaaagaagaccttgagaactatccgtatctgaaattcgctactggcgagagtgatgcaggggtcattgtcagttggaacaccgaaggtccaaagaatgttgaggagaatactaagaccgttgtgctgctaccaaacgctatcagcatcaacccgctgaactggaagcgcgacgagacttatgcgcccgccagcgagaatctggggtcgcttgtggtgaacgaagaaaccggagagcctgagattggtgatcttggtgcggacgcacaggtgaatcttgcccgaggcacaatcgtgacgcacgctaaagtagtcccgatgcccgaggatgccgccaaggtagccgcagagttcttcggacccgatggccgtcatggtgaagactatacatacttctacaacaatatcaaggacaacgttgccaagcgcatcgccaccttcaagacgaatatgcgacaagattag

SEQ ID NO: 6 - protein, 320 aa, unidentified
  REGION 1..320: note = N20O1
METTTDYSQKANWCKFPQITKDVDTFYIFATEYIMGSFEEGAPDYGTLDNQEMIEGMNVEYLVHATTYADSTNVFAPYYRQTGLRFAGDIYKRDGNFEAALIGTPIDDIMAALDYYFEHYNEGRPFIIAGHSQGSALVKVVLMKYFKEHPDYYRRMVAAYVIGYSVTKEDLENYPYLKFATGESDAGVIVSWNTEGPKNVEENTKTVVLLPNAISINPLNWKRDETYAPASENLGSLVVNEETGEPEIGDLGADAQVNLARGTIVTHAKVVPMPEDAAKVAAEFFGPDGRHGEDYTYFYNNIKDNVAKRIATFKTNMRQD

SEQ ID NO: 7 - DNA, 1401 bp, unidentified
  source 1..1401: mol_type = unassigned DNA
  misc_feature 1..1401: note = NYL2
atgaaaattgatcgtggcaccctgctgaaagttctgattctgctgtttaccgttagcggcaattttggttatgaagaaggtgtttttgcacagggtgttccggttgcaaaaaccgatcgtagcggtccgagcctgcagtttgattttccggatatgctggttggtgttgccgaatatgatgaaggtccgaccggcaccaccgttttctattttccgaatggtgttaaaggtgccgttgatatgcgtggtggtagtccgggtgaactgaatgccgaaatcctgcagaatgcatatgaaagccgtatggttaatgccgttgtttttagcggtggtagctggtatggtctgagcgcagcaaccggtgttgcaaatggtattaagaaaatgcgtgcaagcgcaggcaatgttgattttattgccggtgttgttggtgccatcatttttgatgttggtggtcgtcgttttagccgtgttacaccggatgatgcactgggtgaagcagcactgcgtagcgcagaagcaggttggtttccgctgggtgcacgtggtgcaggtcgttttgcaatgcagggtatctattttcatcgtggtccgggtgcagaaaattatgcaaattgggcacatagtggccaaggtggtgcatttcgtaccattggtccgacaaaaattggtgtttttaccgtgattaatgccctgggtagcgttgttgatcgtagtggtaatgttgttcgttgtggtcgtaatgatgcaggtcagaattgtccgagcattgccaataaactgaaaagctttaaaccgatcaatccgcataatggtgatccggcaagtggtccgaccagcaataccaccattacactggttgttaccaatcagaaactgccgtttagcgcactgcagcgtctggcaacccaggttcatagcagcatgggtcgtgcaattcagccgtttgccaccgcacaggatggtgatattctgtatgcagttagcaccgatgaaattgataatccgagcatgagcgcaatggatctgagcgttattgcaagcgaactggcatgggatgcagttctggcaagcgttccgaccattccggaacgtccgaaaccgctggcaagcaaaccggatcgtggtatgattcagaaatttgaaggcagctatcacttttacggtggcggtaaactgattgttaccgttgaagcaaatgcactggcagcacgttttgaaggtaatggtcgcatctatttcgacaaagataaaacctatgcaattaccgcagcacagaatgacctgtttattatcgaaagtccgggtcgtgaagttctgcgttttgatgaaatcgcaggtaaaattaccggtctgaccatgaatcctggtccgtgggaagttcgtagcgcaaaaggtctg

SEQ ID NO: 8 - protein, 467 aa, unidentified
  REGION 1..467: note = NYL2
MKIDRGTLLKVLILLFTVSGNFGYEEGVFAQGVPVAKTDRSGPSLQFDFPDMLVGVAEYDEGPTGTTVFYFPNGVKGAVDMRGGSPGELNAEILQNAYESRMVNAVVFSGGSWYGLSAATGVANGIKKMRASAGNVDFIAGVVGAIIFDVGGRRFSRVTPDDALGEAALRSAEAGWFPLGARGAGRFAMQGIYFHRGPGAENYANWAHSGQGGAFRTIGPTKIGVFTVINALGSVVDRSGNVVRCGRNDAGQNCPSIANKLKSFKPINPHNGDPASGPTSNTTITLVVTNQKLPFSALQRLATQVHSSMGRAIQPFATAQDGDILYAVSTDEIDNPSMSAMDLSVIASELAWDAVLASVPTIPERPKPLASKPDRGMIQKFEGSYHFYGGGKLIVTVEANALAARFEGNGRIYFDKDKTYAITAAQNDLFIIESPGREVLRFDEIAGKITGLTMNPGPWEVRSAKGL

SEQ ID NO: 9 - DNA, 753 bp, unidentified
  source 1..753: mol_type = unassigned DNA
  misc_feature 1..753: note = NYL5
agtccgcatagcggtgttagcctgtttgtttttccggaaggtagcattggtgcagttgatgttcgtggtggtgcaccgggtagccgtgaaaccgaagcactggcaccgggtcatgttaccgaacgtctggatggtctgtttctgagcggtggtagcgcacatggtctggcagcaggtagcatgctgacccagtatctgctgaatcaggatattggtattaaactgcgtccgcatgttccgcctgttccgattgttgccggtgcagttatttatgatctgacaccggaacatctgggtcatgatctgaaacaaatttatgaaggtctggcacaagaagcctgcggtaaactgggcaccaataaagcacagcatcgtgatggtgccggtgcgggtgcacgtgatgcattaggtaaaggtggtctggccaatgcaagctatcgcctggatgatattcatgttggtgttctggttgttgcaaatccggttggtcaggttggtgcacagggtccagttagcctgggtagcaccaaactgcgtgatctgaatctgagcaccaccaatgccggtgccaataccagtattggtgttgttgccaccaatctgaaactgagtcgtctgcagctgaaacgtgttgcaattatggcacatgatggcctggcacgtgcaattcgtccggttcatacaccgcatgatggtgatctgctgtttgcagttagcaccaatcagctgggccacgatctgccggaagcactg

SEQ ID NO: 10 - protein, 251 aa, unidentified
  REGION 1..251: note = NYL5
SPHSGVSLFVFPEGSIGAVDVRGGAPGSRETEALAPGHVTERLDGLFLSGGSAHGLAAGSMLTQYLLNQDIGIKLRPHVPPVPIVAGAVIYDLTPEHLGHDLKQIYEGLAQEACGKLGTNKAQHRDGAGAGARDALGKGGLANASYRLDDIHVGVLVVANPVGQVGAQGPVSLGSTKLRDLNLSTTNAGANTSIGVVATNLKLSRLQLKRVAIMAHDGLARAIRPVHTPHDGDLLFAVSTNQLGHDLPEAL

SEQ ID NO: 11 - DNA, 951 bp, unidentified
  source 1..951: mol_type = unassigned DNA
  misc_feature 1..951: note = NYL9
atgaataataccattctggatgttaatggtctgagcgttggtcaggcacatgataatgaagttaaaaccggtgttaccgttgttctgccggataaaccggttacctgtagcgttgatgttcgtggtggtggtccgggtacacgtgaaaccgaaacactgaatgatggtggtctgattgataaagttcatgccatttgcctgagcggtggtagcgtttatggtctggcagcagcagatggtgtttgtgcatggctgggtaaaaagcgtattggttatgaagttggtccgagtccgattccggttagcccgattgttccgagcgcaattctgtttgataatgcaaatggtggcaataagaactggggtattaatccgccttttcgtagcctgggtattcgtgcatgtgaaagcgttagcaatcatctgaccctgggtcgtattggtgcaggttatggtgcaatgagcggtatctataatggtggtattggtagcgcaagcgaagccattaatgaatttaccgttggtgcactgattgcagcaaatagcgttggtagcccgtttatgcctggcaccaaatgtccgtgggcatggccgtatgaagtgaataacgaatttggtggtaaacgccctccgaaaaattacaaatatgcacatccgcaggataccaaactgaattttctgaatcgtgcaggtcagaataccgttattggtgttgttgcaattgatgcaaatctgacccagaatcagctgaaacgtattgcaattatggcacaggatggtattgccatggccgttcagccgagccatacaccgctggatggtgataccatttttgcagttagcaccggtcataaaagctgtgataatccgaccattctgagcgaactgggtagcgcagcagcacgttgtgttgcacgtagtctgacccgtgcagtttatgaaggtagcaaa

SEQ ID NO: 12 - protein, 317 aa, unidentified
  REGION 1..317: note = NYL9
MNNTILDVNGLSVGQAHDNEVKTGVTVVLPDKPVTCSVDVRGGGPGTRETETLNDGGLIDKVHAICLSGGSVYGLAAADGVCAWLGKKRIGYEVGPSPIPVSPIVPSAILFDNANGGNKNWGINPPFRSLGIRACESVSNHLTLGRIGAGYGAMSGIYNGGIGSASEAINEFTVGALIAANSVGSPFMPGTKCPWAWPYEVNNEFGGKRPPKNYKYAHPQDTKLNFLNRAGQNTVIGVVAIDANLTQNQLKRIAIMAQDGIAMAVQPSHTPLDGDTIFAVSTGHKSCDNPTILSELGSAAARCVARSLTRAVYEGSK

SEQ ID NO: 13 - DNA, 990 bp, unidentified
  source 1..990: mol_type = unassigned DNA
  misc_feature 1..990: note = NYL10
atgagcaaacatcagcctgcaccgcctcgtaccagcagtccgattggtaatgatcatcgtcatcgtccggcagttctgggtgcagccggtgatggttgggttgaatttgatctgcctggtgttgcaattggcaccgcagaatatccggatggtccgaccggtgttaccgttctgagcctgccaggtggtgcacgtaccgcagttgataaacgtggtggtgcagttggtgttagcggtggttttccgtatcatcatgcactgtgttttgccggtggtagcagctttggtctggcagcagcaagcggtgtttatgatgaactgctgcgtcgtaatggttatgcaaccgattttggtagcctgccggttgtgagcggtgcagtggtttatgattttacaccgcgtgataatgcagtttatcctgatgcacagctgggtgccgaaggtctgcgtgcagttcgtaccgatcgtgttgccgttggtcgtgttggtgccggtgtgggtgcaaccgttggtaaagttgataatgcccgtgcagaatggggtggtcagggtgcagcatttgcacgcgttggtgcagcacgtctgctggttctgaccgttgttaatgcaatgggtgttgttgttgatcgtgccggtgaagttgttcgtggtaatctggatccggaaaccggtcgtcgtcgtcatcctgccgaagattatgcagaactgattgcagcagccacaccggatctggcaccgcctggtggtaataccacactgaccgcagtggttaccaatgttcgtatgaccgataccgaactggcacagtttgcaacccaggttcatagcagcatgcatcgtgcaattcagccgtttcataccattgcagatggtgataccctgtttgcactgaccaccgatgaagtgaccgttccggcaccgagcctgcgtctgggcaccgttgcagcagaactggcatgggatgccgttctggcaagcgttgataccccg

SEQ ID NO: 14 - protein, 330 aa, unidentified
  REGION 1..330: note = NYL10
MSKHQPAPPRTSSPIGNDHRHRPAVLGAAGDGWVEFDLPGVAIGTAEYPDGPTGVTVLSLPGGARTAVDKRGGAVGVSGGFPYHHALCFAGGSSFGLAAASGVYDELLRRNGYATDFGSLPVVSGAVVYDFTPRDNAVYPDAQLGAEGLRAVRTDRVAVGRVGAGVGATVGKVDNARAEWGGQGAAFARVGAARLLVLTVVNAMGVVVDRAGEVVRGNLDPETGRRRHPAEDYAELIAAATPDLAPPGGNTTLTAVVTNVRMTDTELAQFATQVHSSMHRAIQPFHTIADGDTLFALTTDEVTVPAPSLRLGTVAAELAWDAVLASVDTP

SEQ ID NO: 15 - DNA, 1098 bp, unidentified
  source 1..1098: mol_type = unassigned DNA
  misc_feature 1..1098: note = NYL11
atgagcctgcctgcactgagcaatgatagctatgcactgcattttccggataatccgaatctggttccgattaccgaaccgcctaaagatggtcgtagcctggatctgagcgattgggcaacctttaaaatcggtattgcagaatatccgagcggtccgaccggtagcaccgtttttatctttagcggtggtaatgcaatttgctatccggatgttcgtggtggtagtccgggtacactgatggcagcaaccggtggtaaaaccgatgcaattgttctgaccggtggttcatgttttggtctggaagcagccaccggtgttaccgcagaactgctggcacagcagggttataccgcagattggcagaccattccgatcacacgtagcgcagttgtttatgacctgagcgcacgtccgaataccatttatcctgataaagcactgggtcgtgcagcagttcgtgcagcccgtccgggtgtttttccgatgggtcgtcgtggtgccggtggtgcagttagcgttggtaaaatggatggtcgtcgcgatatggcagaatgggcaggtcaaggtggtgcatatcgtaactttaatggtgttaaaatcgccgtgtttaccgttgttaatgccgttggtgttattgttgatcgtagcggcaccgttgtgcgtggtcattttgatgccaaaaataaccgtcgtctgagcctggaagaatatagcaccgcatatgcaagcaatagcaccaccgaaagcgttcatccgcgtccggatggtacaccgcgtaataccaccattacactgtttgttaccaatgccaaatttgaaagcgcatttgcagttgaggcctttaaaaacgttgtgaaaagcgtgcatgcaagcatggcacgtgcaattcagccgtttcatctgctgaccgatggtgataccttctttggtgttaccaccaatgaactggatgttgaatggaccgatctgcctccgcctgccggtgataatagcggtcgtgtgaaaatgagcacccaggttctgagctatttaggtggtgaactggcatgggatgcagttctgaatagctttgatgaacgtagcagtaccgttggt

SEQ ID NO: 16 - protein, 366 aa, unidentified
  REGION 1..366: note = NYL11
MSLPALSNDSYALHFPDNPNLVPITEPPKDGRSLDLSDWATFKIGIAEYPSGPTGSTVFIFSGGNAICYPDVRGGSPGTLMAATGGKTDAIVLTGGSCFGLEAATGVTAELLAQQGYTADWQTIPITRSAVVYDLSARPNTIYPDKALGRAAVRAARPGVFPMGRRGAGGAVSVGKMDGRRDMAEWAGQGGAYRNFNGVKIAVFTVVNAVGVIVDRSGTVVRGHFDAKNNRRLSLEEYSTAYASNSTTESVHPRPDGTPRNTTITLFVTNAKFESAFAVEAFKNVVKSVHASMARAIQPFHLLTDGDTFFGVTTNELDVEWTDLPPPAGDNSGRVKMSTQVLSYLGGELAWDAVLNSFDERSSTVG

SEQ ID NO: 17 - DNA, 1020 bp, unidentified
  source 1..1020: mol_type = unassigned DNA
  misc_feature 1..1020: note = NYL12
atgaccgttaatagcgcagcacgtcgtgcaccgcatctgccgagtgatggtcgtggtaatgaaagccagccgctgaccattgttagcaataaaccgggtattccgatcgagtttgattttccgggtgttgaaattggcaccgcagaatatgcagaaggtccgaccggttgtaccctggtttatgttccggcaggcgcacgtaccgcagttgatgaacgtggtggtgcaattggtgcaagcggtcgttatggttataatcatggtattctgctgagcggtggtagcagccatggtctgcaggcagcagccggtgttaccgcagaactgctgcagcgtggtggtaatcagaccggttttgatcagctgccgctgattagcggtgcagttgtttatgattttcctgcacgcggtaatgcaattcatcctgatgttgaactgggtcgtgcagcactgcgtgcagccgttccaggtcgtgccgcaagtggtcgtgttggtgcgggtgttagcgcaaccgttggtaaactggatcacgatcgtagcgaatatgccggtcagggtgttgcatttcgtcgtctgggtgatattcgtgttctggttgttaccattgtgaatgcagttggtgttgttgttgatcgtgatggtagcattgtgcgtggtaattgggatcgtgccagcggtgaacgtcgtctgccggttgttgattatgaacgtgcactggcagcaggtcgtatgcctgcaattccgagcggtaataccaccattaccgcagtggttaccaatgttcgtctgaatgatattgaactgaaccagtttgcaaaacaggtgcatagcagcatgcatcgtgcaattcagccgtttcatacagccgttgatggcgatattctgtttgcactgaccaccgatgaagttagcctgggtgccgaagaagcaagcgttcacggcacctataccgttacaccgaccgcactgggtgcagtggcaagcgaagttgcatgggatgcagttctggcaagcgcaaaa

SEQ ID NO: 18 - protein, 340 aa, unidentified
  REGION 1..340: note = NYL12
MTVNSAARRAPHLPSDGRGNESQPLTIVSNKPGIPIEFDFPGVEIGTAEYAEGPTGCTLVYVPAGARTAVDERGGAIGASGRYGYNHGILLSGGSSHGLQAAAGVTAELLQRGGNQTGFDQLPLISGAVVYDFPARGNAIHPDVELGRAALRAAVPGRAASGRVGAGVSATVGKLDHDRSEYAGQGVAFRRLGDIRVLVVTIVNAVGVVVDRDGSIVRGNWDRASGERRLPVVDYERALAAGRMPAIPSGNTTITAVVTNVRLNDIELNQFAKQVHSSMHRAIQPFHTAVDGDILFALTTDEVSLGAEEASVHGTYTVTPTALGAVASEVAWDAVLASAK

SEQ ID NO: 19 - DNA, 1146 bp, unidentified
  source 1..1146: mol_type = unassigned DNA
  misc_feature 1..1146: note = NYL14
atgagcgcagcagcaccggcaacaggtgcaccgaccactccggcaaccgcaaccgccacagccaccgcaacagcaaccgaaggtagtgatgttccgtttattgatccggcagcaagtgatgcaggtcgtgcagcaccgcatcgtctgacaccgcctccgtattggggtgatcgtgttggtaatgatgatgttgaactggttccgagccatggtgttggtcgtggtctggttgcatttgattttccgggtgttgcagttggcaccgcagaatatgcaaatggtccgaccggtgcaaccgttattaccattgcgaatggtgcacgtaccagcgttgatgcacgtggtggtgcaattggtattattggtcgttatgaagaatttaaccacgccatttgttttgccggtggtagcagctatggtctggcagcggcaaccggtgttgatgaagaactgctggttcgtgaacagggtcgcgttggttggcaggatctgcgtagcgcaagcggtgcagttatttatgatctgggtgcacgtgataccggcattgttccggatgcagcactgggtcgtgccgcactggaagcagcacgtccggatgccattccggttggccgtgttggtgcaggtattgcagcaagcgcaggtaaaattgatctgagccgttgtgaatttaccggtcagggtgcagcatttcgtcagattggtgatattcgtattctggttgtgaccattgttaatccggttggtgttattgttgatcgtgatggtcgtgttgtgcgtggtaattatgatccggaaaccggtgaacgtcgtctgccggttgttgattatgaagcagccctggttgcgggtgaacagccggttaccgcagcaggtaataccaccattaccgcagttgttaccaatgtgaaactggatgataaagagctgcgtctgtttggtcgtcaggttcatagcagcatgcatcgtgcaattcagccgtttcataccaatcatgatggtgataccctgtttgcactgaccaccgatgaaattgcactgcctccggcaggtagcacccgtctgggcgaacaggcagttaatagcattgcactggcagcagttgcaagcgaactggcatgggatgcagttctggaagccggtcgt

SEQ ID NO: 20 - protein, 382 aa, unidentified
  REGION 1..382: note = NYL14
MSAAAPATGAPTTPATATATATATATEGSDVPFIDPAASDAGRAAPHRLTPPPYWGDRVGNDDVELVPSHGVGRGLVAFDFPGVAVGTAEYANGPTGATVITIANGARTSVDARGGAIGIIGRYEEFNHAICFAGGSSYGLAAATGVDEELLVREQGRVGWQDLRSASGAVIYDLGARDTGIVPDAALGRAALEAARPDAIPVGRVGAGIAASAGKIDLSRCEFTGQGAAFRQIGDIRILVVTIVNPVGVIVDRDGRVVRGNYDPETGERRLPVVDYEAALVAGEQPVTAAGNTTITAVVTNVKLDDKELRLFGRQVHSSMHRAIQPFHTNHDGDTLFALTTDEIALPPAGSTRLGEQAVNSIALAAVASELAWDAVLEAGR

SEQ ID NO: 21 - DNA, 948 bp, unidentified
  source 1..948: mol_type = unassigned DNA
  misc_feature 1..948: note = NYL18FL
gcagaaagcaatgataccctgcatccggttgcacgtaccgaagttgatgcaaaacgttggctggaagttgattttcctggtctggaaattggtgttgcagaatatccggaaggtccgaccggttgtaccgtttttcattttccggaagttgcaagcatgcatattgatgttcgtggtggtagtccgggtattaatggtgaacatctgaccctgaccaatgcaatttgttttgccggtggtagcctgtatggcctggaagcaagcaccggtgtggcagcagaactgtttggtcgtcgtggttatagcaccaaatggatggatattgcactggttgccggtgcaattatctttgattatggtcgtcgcgataatgccgtgtatcctgataaagatctgggtcgtgcagcagttcgtgcactgcgtccgggtcgttttccgcagggtccgcatggtgcaggttgtagcgcaaccgcaggtcatggtgccaccgcagaactggcaggtcaaggtggtgcaagccgtcgtattggtccgacacgtgttgcagtttttaccgttattaatgcctatggtgccattgttgatcgtaccggtgatgtggttcgtggtaatcgtgatccgaaaaccggtcgtcgtctgcgtgcagaacgtattgttcaagaaaccggtgcatatgatccgattgcagcacgtgcccgtggtgttacccagaataccaatctgacactggtggcaaccgatcagaaactggatccgctgcagctgcgtagcctggcacgtcaggttcatagcagtctggcacgtgcagtgcagccgtttcatacccgttgggatggtgatattctgtttgcactgagcacccagcagaccaccaatgaagcactggatgaagttagcctgggtgttgttgcaagcgaactggcatgggatgcagcactggcaagcttt

SEQ ID NO: 22 - protein, 316 aa, unidentified
  REGION 1..316: note = NYL18FL
AESNDTLHPVARTEVDAKRWLEVDFPGLEIGVAEYPEGPTGCTVFHFPEVASMHIDVRGGSPGINGEHLTLTNAICFAGGSLYGLEASTGVAAELFGRRGYSTKWMDIALVAGAIIFDYGRRDNAVYPDKDLGRAAVRALRPGRFPQGPHGAGCSATAGHGATAELAGQGGASRRIGPTRVAVFTVINAYGAIVDRTGDVVRGNRDPKTGRRLRAERIVQETGAYDPIAARARGVTQNTNLTLVATDQKLDPLQLRSLARQVHSSLARAVQPFHTRWDGDILFALSTQQTTNEALDEVSLGVVASELAWDAALASF

SEQ ID NO: 23 - DNA, 933 bp, unidentified
  source 1..933: mol_type = unassigned DNA
  misc_feature 1..933: note = NYL20FL
attagcaatgcaaataccgcactgaccgttgttacaccggcagaaaccccgagcctgtcatttgatctgccggaactgaccgtgggtattgcagaatatccggaaggtccgaccggttgtaccgttctgcgttttgcacgtgccctgcgtaccgcagttgatgttcgtggtggttgggttggtatgacccaggcatttaccgtttgtgatgcaatttgtctgaccggtggtagcctgattggtctggaaagcgtttatggtgttatggcaggtattctggcagaacgtgaatatagcctggcacagggtaatccggttgttaatggtgcagtgatttatgattatcgctttcgtgccaataccatccatccggatatggcactgggtcgtgcagcactggatgcagcacgtaccggtgcatttccgctgggtagccgtggtgcaggtcgtagcgcaagcgttggtaaagtgtatgattattcacgtgcagaaagcggtggtcagggtgcagcatatcgtcagattcgtgatgttcagctggcagtttttaccgtggttaatgcactgggtgttattgttgatcgtgcaggtcaggttgtgcgtggtaatctggatccgagcaccggtcgtcgtagcgattttgcacatgaagcagcaaccctgacctatgatagcgccagccgtagcaccgttccggaaaacaccacactgacagttgtggttaccaatcagaaactggatgatttttcactgggtcagctgggtcgtcaggttcatggtagcatggcacgcgcaattcagccgtttcataccgcacgtgatggtgatgttctgtatgcagttaccaccgatgaagttgatggtctgccgctgaccagcctgggtatcctggcaggcgaactggcatgggatgcagttctgagcgcagttgaa

SEQ ID NO: 24 - protein, 311 aa, unidentified
  REGION 1..311: note = NYL20FL
ISNANTALTVVTPAETPSLSFDLPELTVGIAEYPEGPTGCTVLRFARALRTAVDVRGGWVGMTQAFTVCDAICLTGGSLIGLESVYGVMAGILAEREYSLAQGNPVVNGAVIYDYRFRANTIHPDMALGRAALDAARTGAFPLGSRGAGRSASVGKVYDYSRAESGGQGAAYRQIRDVQLAVFTVVNALGVIVDRAGQVVRGNLDPSTGRRSDFAHEAATLTYDSASRSTVPENTTLTVVVTNQKLDDFSLGQLGRQVHGSMARAIQPFHTARDGDVLYAVTTDEVDGLPLTSLGILAGELAWDAVLSAVE

SEQ ID NO: 25 - DNA, 1029 bp, unidentified
  source 1..1029: mol_type = unassigned DNA
  misc_feature 1..1029: note = NYL24FL
gcatttgcagcactgctggcaccgctgcagcctgcaccggcatgtgcacagggtcaagattttcgtccgattgttaatcagggtgaacgcattctggaatttgattggagcgatattcgtgttggcaccgcagaatatgaagaaggtccgacaggcgcaaccgttattagctttggtcgtcgtgcactggttgcaatggatgcacgtggtggtggtccgggtacagttaatagcgattatattggtctgggttatgatctggcagaactggatgcagttgtttttagcggtggtagctggtatggtctggaaaccgttaccgcagttgcaagcgcaatgaaagatgatggttatcgtgatggtagcgcatttaccgatccgcctaatattgccctggttaccggtagcattatctttgattatggtccgcgtcgtctgaacgaaatttatccggattcacgtctggcacaggcagcatatcgtgcagcacagcctggtcgttttgcactgggtgccgaaggtgcaggtcgttttaccattagtggtggtttctttggttgtaatgcatatagcggtcaaggtggtgcatttcgtcagattggtgaactgaaaattgccgtttttaccgtggttaatgcctatggtgccgttgttgatcgtcagggtcgtattccgacctgttatagtggtccgaattggcctcctggtgcaggtattaccgatctgatgcaggcatatcctgatagcgcacgtcctggttggggtgaaaatgcacgtcgtcagaataccaccgttagcctggttgttattaatcagcgtatggcaccagccgaactgcatcgtctggcagttcaggttcataccagcatggcacgtggtattcagccgtttgcaaccctgtatgatggtgatgttctgtatgcagcaagcacccaagagctggatctgaatgaaagcggtctgagcagcgttgaaattagcgttattgcaagcgaactgatgtgggatgcaattctgagcgcagcaccg

SEQ ID NO: 26 - protein, 343 aa, unidentified
  REGION 1..343: note = NYL24FL
AFAALLAPLQPAPACAQGQDFRPIVNQGERILEFDWSDIRVGTAEYEEGPTGATVISFGRRALVAMDARGGGPGTVNSDYIGLGYDLAELDAVVFSGGSWYGLETVTAVASAMKDDGYRDGSAFTDPPNIALVTGSIIFDYGPRRLNEIYPDSRLAQAAYRAAQPGRFALGAEGAGRFTISGGFFGCNAYSGQGGAFRQIGELKIAVFTVVNAYGAVVDRQGRIPTCYSGPNWPPGAGITDLMQAYPDSARPGWGENARRQNTTVSLVVINQRMAPAELHRLAVQVHTSMARGIQPFATLYDGDVLYAASTQELDLNESGLSSVEISVIASELMWDAILSAAP

SEQ ID NO: 27 - DNA, 1068 bp, unidentified
  source 1..1068: mol_type = unassigned DNA
  misc_feature 1..1068: note = NYL25FL
gttctggcaaccgcaatgctgaccgcatgtgcagcactgccgcagagcgcagttgcagaaccgggtagcgttgcacaagaagatctgcctgcactgattaatgatggttttgatagcgaagcagcactgacctttgattggccgatgctggaaattggcaccggtcagtatgaagatggtccgaccggtctgacagttctgaaatttggtaaaaaggttaatgcagccgttgatagccgtggtggtggtccgggtacagttaatgcaaaatatctggaactgggttacaatcagccggaactggatgcaattgttctgagcggtggtagctggtatggtctggaagcagttaccgcagcaaataccgcactgctggatgatcagtatcgtggtggtcgttgggataatgttggtctggttgttggtagcatcatctatgattttggtggtcgccgtctgaatgaaatttatcctgataaacgtctggcacagctggcactgcgtgcagccgaaccgggtgtttttccgaatggtgcatatggtgcaggtcgtagcgcaatgaatggtggtatttttggttgtaatgcacgtagcggtcagggtagcgcatttcgtcagattggtgatgttaaaattgccgtttttaccgttgttaatgcactgggtagcgtgaccgatcgtgaaggtaatgttgttgcatgtcataaaggtagcaattggcctgaaaatctgaaagcaaccgatctgatgcgtgccgcaccggatagcgttcagcctggttggaatggtgatgaagaaagtccgggtgcagaacgtaaaaacaccacaattagcgttgttgtgaccaatcgtaccatgagtccggcagaactgcagcgtctggcagttcaggttcataccagcatggcacgtggtattcagccgtttgcaagcgaatttgatggtgatgtgctgtatgcagttagcaccaatgaagttgaaaccggtccgggtcagaccattccgagcatggaaatcggtgttgcagcaagcgaactgatgtgggatgccattctgaatgcagttccg

SEQ ID NO: 28 - protein, 356 aa, unidentified
  REGION 1..356: note = NYL25FL
VLATAMLTACAALPQSAVAEPGSVAQEDLPALINDGFDSEAALTFDWPMLEIGTGQYEDGPTGLTVLKFGKKVNAAVDSRGGGPGTVNAKYLELGYNQPELDAIVLSGGSWYGLEAVTAANTALLDDQYRGGRWDNVGLVVGSIIYDFGGRRLNEIYPDKRLAQLALRAAEPGVFPNGAYGAGRSAMNGGIFGCNARSGQGSAFRQIGDVKIAVFTVVNALGSVTDREGNVVACHKGSNWPENLKATDLMRAAPDSVQPGWNGDEESPGAERKNTTISVVVTNRTMSPAELQRLAVQVHTSMARGIQPFASEFDGDVLYAVSTNEVETGPGQTIPSMEIGVAASELMWDAILNAVP

SEQ ID NO: 29 - DNA, 858 bp, unidentified
  source 1..858: mol_type = unassigned DNA
  misc_feature 1..858: note = SOIL1
atgcctcgtaataccacactgaccgcactgcctggtgttcgtgttggtcatgcaaccgatcgtgaacgtcgtaccggcaccaccgttgttctggttgaaccgcctgcaatggcatttgcagatatgcgtggtggttggcctggtagctttgataccgcagcaagcgatctgggtaaaacctttattggtcgtcatgcactgtttctgaccggtggtgatgtttatggttatgacgcagcaattggtattcgtcgttatctgctggcacgtggtctggcacgtccgcgtggtggcggtaaaatgcctgcaattatgggcaccaacatttatgatgttcattttggtccgctggaaggtcgtgattataccgcactgggtgaacgtgcatgtggtgcagccagcgcacgtccggttccgcagggtacagttggtgcaggtacaggtgcagcagttggtccgtttgttgatcgtgcaggtctggcaggcaccaaaggtggtctgggtagcagcgcagcacgcgttggtcgttggaccgttggcaccattgcagttaccaattgtgttggtaatgttgttgatgttgcagcaggtcgtaccattgccggtacacatcgtcgtggtagcccgacctttctggaaatggatgatgttctggatgcctatctgcgtcgtagcgcagttcgtagcggtacaaccattggtgttgttggcaccgatgcaccgctggatcatgaacagctggcacgcctggtggaactggcacatgatggttttgcactggcaattcgtccggcacatctgagcaccgatggtgataccattctgggtttttgtaccggtcgccgtccggcaaaacgt

SEQ ID NO: 30 - protein, 286 aa, unidentified
  REGION 1..286: note = SOIL1
MPRNTTLTALPGVRVGHATDRERRTGTTVVLVEPPAMAFADMRGGWPGSFDTAASDLGKTFIGRHALFLTGGDVYGYDAAIGIRRYLLARGLARPRGGGKMPAIMGTNIYDVHFGPLEGRDYTALGERACGAASARPVPQGTVGAGTGAAVGPFVDRAGLAGTKGGLGSSAARVGRWTVGTIAVTNCVGNVVDVAAGRTIAGTHRRGSPTFLEMDDVLDAYLRRSAVRSGTTIGVVGTDAPLDHEQLARLVELAHDGFALAIRPAHLSTDGDTILGFCTGRRPAKR

SEQ ID NO: 31 - DNA, 909 bp, unidentified
  source 1..909: mol_type = unassigned DNA
  misc_feature 1..909: note = MARINE2
tatccgggtgaaaataatctgattaccgatgtggatggtattctggttggtaatgccgaaaatgaaattgttggcaccggcaccaccgttctgattccgcctgaaggtagcaccgcaagcgcagatgttcgtggtggtgcaccgggtacacgtgaagttggtgcactgaatccgtataatctggttgataccattgatgccattgttctgagcggtggtagcacctggggtttagatgcagcaagcagcgttgcaaactttattggtaaaaccggtcgtggttttaaaagcggcaatgatgttaaaaatgttccgatggttccgggtgcaattctgtttgatctgaataatggtggcaataaagattggggtagcgaaagcccgtatcataaactgggtgttgaagcaagccagaaagcatcacagaaatttgatctgggtaatgttggtgcaggttttggtgcaaaagcaggtagcctgaaaggtggtctgggtagcgcaagcattgaaaacaaatttggtctgaaaattggcggtctgtttgccgttaatagctttggtagcgttgttaatagcgataccggtcagttttggggtgcaagtgatgaactgaataacgaatttggtaatctgggtgctccgcagaaactgccgaccgatgttctggcaggtagcgccattggtcatgcactggaaaagaataataccaccattggtattgttgccaccaatgcaaaactggatagcaaaaccgcaaaacgcattgcaattatgagccataccggcatgagccgtgcaattcgtccgattcatacaccggttgatggtgatgttatttttgttgttagcaccaacacctatgataaagaactgagctatcgcgaaatcaacgaaattggtgaactgggc

SEQ ID NO: 32 - protein, 303 aa, unidentified
  REGION 1..303: note = MARINE2
YPGENNLITDVDGILVGNAENEIVGTGTTVLIPPEGSTASADVRGGAPGTREVGALNPYNLVDTIDAIVLSGGSTWGLDAASSVANFIGKTGRGFKSGNDVKNVPMVPGAILFDLNNGGNKDWGSESPYHKLGVEASQKASQKFDLGNVGAGFGAKAGSLKGGLGSASIENKFGLKIGGLFAVNSFGSVVNSDTGQFWGASDELNNEFGNLGAPQKLPTDVLAGSAIGHALEKNNTTIGIVATNAKLDSKTAKRIAIMSHTGMSRAIRPIHTPVDGDVIFVVSTNTYDKELSYREINEIGELG

SEQ ID NO: 33 - DNA, 18 bp, synthetic construct
  misc_feature 1..18: note = M13-Forward Primer
  source 1..18: mol_type = other DNA
tgtaaaacgacggccagt

SEQ ID NO: 34 - DNA, 17 bp, synthetic construct
  misc_feature 1..17: note = M13-Reverse Primer
  source 1..17: mol_type = other DNA
caggaaacagctatgac

SEQ ID NO: 35 - DNA, 40 bp, synthetic construct
  misc_feature 1..40: note = cl5en2 Forward Primer
  source 1..40: mol_type = other DNA
gaaaacctgtacttccagggtatggcggattatgcgaaag

SEQ ID NO: 36 - DNA, 46 bp, synthetic construct
  misc_feature 1..46: note = cl5en2 Reverse Primer
  source 1..46: mol_type = other DNA
tgacactatagaatactcaagcttacaccacaaaacggctaaattc

SEQ ID NO: 37 - DNA, 24 bp, synthetic construct
  misc_feature 1..24: note = ePCR reaction mix forward primer
  source 1..24: mol_type = other DNA
ggtaccgaaaacctgtacttccag

SEQ ID NO: 38 - DNA, 30 bp, synthetic construct
  misc_feature 1..30: note = ePCR reaction mix reverse primer
  source 1..30: mol_type = other DNA
ctgggatttaggtgacactatagaatactc

(19) United States
(12) Patent Application Publication
(10) Pub. No.: US 2026/0055398 A1
(43) Pub. Date: Feb. 26, 2026

(54) IMPROVED POLYMER DEGRADING ENZYMES

(71) Applicant: CAMBRIDGE ENTERPRISE LIMITED, Cambridge (GB)

(72) Inventors: Florian HOLLFELDER, Cambridge (GB); Mariana RANGEL PEREIRA, Cambridge (GB); Josephin HOLSTEIN, Cambridge (GB); Tomasz KAMINSKI, Cambridge (GB); Philip MAIR, Cambridge (GB); Fabrice GIELEN, Cambridge (GB)

(21) Appl. No.: 18/866,427

(22) PCT Filed: May 18, 2023
(86) PCT No.: PCT/GB2023/051312

§ 371 Date: Nov. 15, 2024

(30) Foreign Application Priority Data
May 18, 2022 (GB) 2207298.7

(51) Int. Cl. C12N 15/10; C40B 30/08

(52) U.S. Cl. CPC C12N 15/1072; C40B 30/08

(57) ABSTRACT

The invention relates to methods of identifying enzymes and methods of screening for optimised enzymes for degrading polymers such as plastics. The invention also relates to polymer degrading enzymes and nucleic acids encoding these enzymes as well as expression cassettes and host cells comprising the nucleic acids. The invention also relates to uses of these enzymes or the host cells comprising the nucleic acids encoding these enzymes to degrade polymers such as plastics. Lastly, the invention also relates to the crystal structure of plastic degrading enzymes and uses of these structures.

### FIELD OF THE INVENTION

The present invention relates to a fast throughput method for identifying and optimising novel polymer degrading enzymes, for example plastic degrading enzymes. The invention also relates to novel polymer, e.g. plastic degrading enzymes, crystal structures of these enzymes and methods of using the crystal structures.

### BACKGROUND

460 million tons of plastics are produced annually worldwide with 90% produced from fossil fuels. Around 79% accumulates in landfill or in the natural environment.

Poly(ethylene terephthalate) (PET) is the most abundant polyester plastic, with almost 70 million tons manufactured annually worldwide for use in textiles and packaging. If this plastic could be recycled, this would reduce fossil fuel consumption and landfill use.

Currently, only a small number of PETases (enzymes which degrade PET into its constituent monomers) are known. These were found by low throughput methods such as agarose-based screenings with soluble enzymes. Although these screening techniques were extensively used over recent decades to access the vast diversity provided by metagenomic libraries, the screening campaign can be laborious, cost and time-intensive.

### SUMMARY OF THE INVENTION

It is important to find more efficient PETases and generally more efficient plastic degrading enzymes as well as other enzymes which degrade polymers which accumulate in the environment.

The inventors have solved this problem in the field using a novel method to find and improve enzymes which degrade polymers, for example plastics such as PET.

Therefore, in a first aspect there is provided a method for identifying a plastic degrading enzyme or optimising a plastic degrading enzyme, the method comprising:
a) encapsulating a gene library into a plurality of microfluidic droplets, each microfluidic droplet comprising at least one gene and each microfluidic droplet additionally comprising:
i) an expression system for expressing the gene; and
ii) a plastic particle;
b) detecting degradation of the plastic particle in the microfluidic droplet; and
c) selecting microfluidic droplets where plastic degradation has been detected.

Detecting degradation may optionally be by detecting a reduction in light scattering by the plastic particle.

Optionally, the gene library is:
a) a metagenomic library; or
b) a directed evolution library.

If the gene library is a metagenomic library, optionally the method further comprises:
a) identifying the plastic degrading enzyme; and optionally
b) making a directed evolution library from the plastic degrading enzyme and repeating the method of the first aspect with the directed evolution library.

The method for optimising described above may optionally be preceded by a screening method, wherein the screening method comprises:
a) encapsulating a gene library into a plurality of microfluidic droplets, each microfluidic droplet comprising at least one gene and each microfluidic droplet additionally comprising:
   i) an expression system for expressing the gene; and
   ii) a soluble plastic mimic;
b) detecting cleavage of the soluble plastic mimic in the microfluidic droplet; and
c) selecting microfluidic droplets where cleavage has been detected.

In a further aspect, there is provided an esterase having at least 70% or more sequence identity to:
a) SEQ ID NO. 2; or
b) SEQ ID NO. 4; or
c) SEQ ID NO. 6.

In a further aspect, there is provided an isolated and/or recombinant nucleic acid encoding any of the plastic hydrolysing enzymes described herein; an expression cassette or vector comprising the isolated or recombinant nucleic acid; or a host cell comprising the isolated and/or recombinant nucleic acid or the expression cassette or the vector.

In a further aspect, there is provided use of any of the esterases described herein to degrade plastic.

In a further aspect, there is provided a crystal of an esterase as described herein and in accordance with the claims.

In a further aspect, there is provided a computer-readable data storage medium encoded with the atomic co-ordinates of the residues in Tables 2 or 3, or the atomic co-ordinates in FIG. 16 or 17.

In a further aspect, there is provided use of atomic co-ordinates with a root mean square deviation of less than 5Å from the backbone atoms of the residues in Table 2 or Table 3, or less than 5Å from the atomic co-ordinates in FIG. 16, optionally the co-ordinates of amino acids 12-325 of FIG. 16, or FIG. 17, optionally the co-ordinates of amino acids 2-267 of FIG. 17, to:
a) optimise the plastic degrading activity by computational design;
b) optimise the thermostability by computational design;
c) design a plastic hydrolysing enzyme; or
d) phase structural biology data obtained from protein.

In a further aspect there is also provided:
a method for screening for an optimised plastic degrading enzyme, the method comprising:
a) encapsulating a gene library with variant gene sequences of the plastic degrading enzyme into a plurality of microfluidic droplets, each microfluidic droplet comprising at least one variant gene sequence of the plastic degrading enzyme and each microfluidic droplet additionally comprising:
   i) an expression system for expressing the gene; and
   ii) a plastic particle;
b) detecting degradation of the plastic particle in the microfluidic droplet; and
c) selecting microfluidic droplets where plastic degradation has been detected.

In a further aspect there is also provided:
a method for identifying a plastic degrading enzyme, the method comprising:
a) encapsulating a gene library into a plurality of microfluidic droplets, each microfluidic droplet comprising at least one gene and each microfluidic droplet additionally comprising:
   i) an expression system for expressing the gene; and
   ii) a plastic particle;
b) detecting degradation of the plastic particle in the microfluidic droplet; and
c) selecting microfluidic droplets where plastic degradation has been detected.

In a further aspect there is also provided:
a hydrolase, optionally an esterase, having at least 70% or more sequence identity to:
a) SEQ ID NO. 2, optionally wherein the enzyme comprises any one or more of the following mutations in SEQ ID NO. 2:
i) A9S; and/or
ii) A118E; and/or
iii) A19T;
or
b) SEQ ID NO. 4;
or
c) SEQ ID NO. 6.

In a further aspect, there is also provided:
a polypeptide with 100% sequence identity to SEQ ID NO.s 2, 4 or 6, optionally with 100% sequence identity to SEQ ID NO. 2 except from the following mutations:
a) A9S; and/or
b) A118E; and/or
c) A19T; or
d) A118E and/or F221S and/or S235G; and/or
e) A118E and/or F221S; and/or
f) A19T and/or A118E and/or F221S; and/or
g) F221S.

In a further aspect, there is also provided a hydrolase having at least 70% identity to SEQ ID NO.s 8, 10, 12, 14, 16, 18, 20, 22, 24, 26, 28, 30 or 32 or a polypeptide having 100% identity to SEQ ID NO.s 8, 10, 12, 14, 16, 18, 20, 22, 24, 26, 28, 30 or 32.

### DETAILED DESCRIPTION

### The Method

### Encapsulating

Encapsulation in a microfluidic droplet may be carried out with a droplet generation module.

### Gene Library

By gene library is meant 2 or more genes. i.e. two or more different nucleic acid sequences. The gene library may be in the form of a metagenomic library. That is, a library comprising sequences from environmental samples. This type of gene library may comprise genes which differ substantially from each other both structurally and functionally as these are random sequences collected from environmental samples.

Alternatively, the gene library may be in the form of a directed evolution library. In a directed evolution library, the gene library is derived from one original nucleic acid sequence which has been mutated to form variants of this original gene sequence, i.e. it is a variant library or randomised DNA library. Two or more genes from this library may include the original gene for example and a mutant variant with one or more nucleic acid changes to the original sequence.

The one or more genes in each microfluidic droplet are from this gene library. The genes may be cloned into a vector in the library. That is the library may comprise a plurality of vectors, each comprising one or more genes. Each gene in the library may also have a promoter. That is, the gene is part of an expression cassette.

### Expression System

By expression system is meant the components required to transcribe and translate the gene into an enzyme.

The expression system may be in vitro translation expression system, or a cell where the cell comprises an expression system. For example, a bacterial cell.

Lysis buffer to lyse the cell may also be encapsulated if the expression system is a cell.

Where a cell is used to express the gene, the gene may be cloned into a vector and transformed into the cell for expression. That is, the expression system and gene are for example a bacterial cell comprising a vector, the vector comprising a gene cloned into the vector. The vector may comprise a promoter for expressing the gene.

After encapsulation, the droplets may be incubated to allow the gene to be expressed (and/or the plastic particle to be degraded). For example, incubated for a period of time ranging from 30 seconds to 4 weeks. For example, 1-5 hours, 24 hours or 48 hours. Incubations may be done on-chip if a short incubation time is used. Lysis buffer may be added to the droplet after the incubation time, for example by picoinjection. Alternatively, the gene may be expressed in a cell prior to incorporation of the cell in the droplet. In this way, lysis buffer can be incorporated into the droplet with the cell.

### Plastic Particle

The plastic particle is solid. The particle may be 50-1000 nm in size. For example, 100-200 nm, for example, 100, 200, 300, 400, 500, 600, 700, 800, 900 or 1000 nm or any range from these sizes. Dynamic light scattering may be used to verify the uniform size of the particle. The polydispersity of the particles may be below 1. That is, the particles are monodisperse. The crystallinity of the particles may be between 1-40% (portion of sample that is crystalline, i.e. 8% means 8% of the sample is crystalline and the rest is amorphous). For example, 1, 2, 3, 4, 5, 6, 7, 8, 10, 15, 20, 25, 30, 35 or 40% or a range of any of these values. This can be measured by Differential Scanning calorimetry (DSC). With this technique the glass transition temperature (Tg), the melting temperature (Tm) and the heats of melting ΔHm and of cold crystallization ΔHc can be determined. By comparing to the heats of melting for a 100% crystalline sample, the percentage crystallinity can be calculated. The microfluidic droplet may contain one or more plastic particles.

The particle may be made by precipitation and solvent evaporation of the plastic substrate. The plastic substrate may be in amorphous or crystalline form prior to precipitation and solvent evaporation.

Where the plastic substrate is for example PET or polyamide, the solvent may be 1,1,1,3,3,3-hexafluoro-2-propanol or a mixture of chloroform and trifluoroacetic acid. Where the plastic substrate is PCL, PES or PLA, the solvent may be dichloromethane (DCM). Where the plastic substrate is polyvinyl chloride (PVC), tetrahydrofuran (THF) may be used as solvent.

When suspending the nanoparticle in the microfluidic droplet, the nanoparticle may be suspended in Tween (polysorbate), for example Tween-80. The resulting concentration in the droplet prior to scattering may be 0.1-0.5% Tween (Tween to volume of droplet) or for example, 0.05-1%. Other surfactants may also be used, for example, Triton, SDS or poly(vinyl alcohol).

### Detecting Degradation

Detection of degradation may be done by light scattering. If the droplet contains an enzyme with plastic degrading activity, the light scattering from the particle will be less compared with a droplet which does not contain an enzyme with plastic degrading activity. By degradation is meant breaking up of the polymer particle into constitute monomers.

### Selecting

By selecting is meant selecting the droplets which contain an enzyme with plastic degradation activity and isolating them from droplets which have not exhibited plastic degradation.

### Identifying the Enzyme

The plastic degrading enzyme may be identified from the gene sequence in the droplet. The identity of the gene may be verified by sequencing of the nucleic acid comprising the gene in the droplet. This can be done by recovering and purifying the DNA of the gene from the droplet, followed by sequencing the DNA.

### Screening Method

Optionally, a first screening method may be carried out to detect enzymes which may have plastic degradation properties. The first screening method uses a soluble plastic mimic. When cleaved, the soluble plastic mimic releases a signal that can be detected. For example, when screening for a plastic degrading enzyme which degrades a plastic comprising ester bonds, the soluble plastic mimic may be an ester (rather than a polymer) covalently bonded to a signalling molecule, for example fluorescein. For example, the soluble plastic mimic may be a fluorescein ester, for example, fluorescein dihexanoate. When this soluble plastic mimic is cleaved, an increase in fluorescence may be detected.

A gene sequence of a plastic degrading enzyme may be identified from the microfluidic droplets where cleavage has been detected. The enzyme encoded by this gene sequence may then be optimised as described below.

### Optimising the Enzymes Further

By screening for an optimised enzyme is meant searching for and optionally identifying sequence variants (mutants) which have polymer, e.g. plastic, degrading activity. The method may further comprise selecting an optimised variant. The variant may be improved from the original plastic degrading enzyme in a variety of ways, for example, improved functionally, compared to the original enzyme. The improved function may be better polymer degrading activity or thermal stability or other functional improvement.

By directed evolution is meant mutation of a nucleotide sequence encoding an enzyme. The resulting mutants can then be screened to find improved functional mutants.

### The Microfluidic Device

The microfluidic device(s) used are designed to first encapsulate the library (insert composed by either a single gene or multiple genes), plastic particle and expression system for the gene in the microfluidic droplet (droplet generation module); and second to detect and sort droplets (scatter sorter module).

These functions may be split between two devices allowing incubation of the droplet and degradation to occur, or they may be part of one device.

The droplet generation module may comprise a droplet generation junction in fluid communication with one or more channels, the channel(s) adapted to flow the gene, the expression system, the plastic particle and a partitioning fluid, e.g. oil, into the droplet generation junction. The droplet generation junction is adapted to encapsulate the gene, expression system and plastic particle in the partitioning fluid.

After encapsulation and a period of incubation to let the enzyme-catalysed reaction proceed, the microfluidic droplets are sorted. The sorter may comprise a bifurcated sorting junction downstream of the droplet generation junction, the bifurcated sorting junction in fluid communication with a first exit channel and a second exit channel wherein the bifurcated sorting junction is adapted to sort the droplets into a first droplet set which exits via the first exit channel and a second droplet set which exits via the second exit channel.

The mechanism may be by detecting fluorescence or by detecting light scattering. Detection may be in comparison to a control droplet which does not have a gene present (or a gene codifying a not active enzyme). If the detection is light scattering, the light scattering will be reduced if the plastic has been degraded. If the detection is by fluorescence, the fluorescence signal will increase with enzymatic activity.

The optical signal for light scattering may be detected using 2 fibres at approximately 90° angle to each other (see FIG. 2A). Additionally, the droplet channel leading into the detection point may be at an angle of approximately 90° to the detection fiber and 180° to the incident light. This enables the fibers to be inserted close to the microfluidic channel.

The fiber providing the incident light may be smaller in diameter than the droplet and the detection fiber.

### The Enzymes

### Plastic Degrading

By plastic degrading is meant the ability to break the plastic polymer down into constituent parts.

The enzyme may be a hydrolase. For example the enzyme may belong to the enzyme class EC 3. The degradation may be by catalysing the hydrolysis of polyesters into an acid and an alcohol. That is, the enzyme is for example an esterase or a lipase or a cutinase. The enzyme may belong to the class of hydrolases classified as EC 3.1.

The degradation may be by catalysing the hydrolysis of polyamides releasing amines and carboxylic acids. That is, the enzyme is a nylon hydrolase or nylonase. They may belong to the class of hydrolases classified as EC 3.5.1.117.

For example, SEQ ID NO.s 2, 4 and 6 are hydrolases, for example esterases and may be classified further as PETases. For example, SEQ ID NO.s 8, 10, 12, 14, 16, 18, 20, 22, 24, 26, 28, 30 and 32 are hydrolases and may be further classified as nylon hydrolases.

### Plastics

By plastic is meant a synthetic material comprising a polymer that can be moulded, extruded, pressed or otherwise shaped into a rigid or slightly elastic form.

The plastic may be a polyester, a polyamide; a polyethylene, polyurethane or polyolefin.

Plastics may include any one of the following: polyethylene terephthalate (PET), polytrimethylene terephthalate (PTT), polyethylene succinate (PES), polybutylene terephthalate (PBT), polyethylene isosorbide terephthalate (PEIT), polylactic acid (PLA), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly (ethylene adipate) (PEA), polyethylene naphthalate (PEN), polystyrene (PS), polypropylene (PP), polyvinyl chloride (PVC), polyurethane (PUR), e.g. elastane, polyamides (PA), polycarbonate (PC) and blends/mixtures of any of the polymers described herein. The particle may comprise or consist of any one of the above plastics or blends/mixtures.

The plastic may be a polyamide such as nylon. The nylon may be nylon 6 or nylon 12, or nylon 6,6

### Recombinant

By recombinant is meant an exogenous nucleic acid sequence which is not native to the cell in which the nucleic acid is being expressed.

### Sequence Listing

TABLE 1
SEQ ID Numbers of enzymes (used in sequence listing)

| Name | DNA SEQ ID NO. | Amino acid SEQ ID NO. |
|---|---|---|
| C15EN2 | 1 | 2 |
| RR1102 | 3 | 4 |
| N20 | 5 | 6 |
| NYL2 | 7 | 8 |
| NYL5 | 9 | 10 |
| NYL9 | 11 | 12 |
| NYL10 | 13 | 14 |
| NYL11 | 15 | 16 |
| NYL12 | 17 | 18 |
| NYL14 | 19 | 20 |
| NYL 18FL | 21 | 22 |
| NYL20FL | 23 | 24 |
| NYL24FL | 25 | 26 |
| NYL25FL | 27 | 28 |
| SOIL1 | 29 | 30 |
| MARINE2 | 31 | 32 |
| M13 Forward Primer | 33 | — |
| M13 Reverse Primer | 34 | — |
| C15EN2 Forward Primer | 35 | — |
| C15EN2 Reverse Primer | 36 | — |
| ePCR Reaction Mix | 37 | — |
| Forward Primer | | |
| ePCR Reaction Mix | 38 | — |
| Reverse Primer | | |

### Sequence Identity

Sequence identity may be calculated using any suitable software such as BLAST (Altschul, S. F., Gish, W., Miller, W., Myers, E. W. & Lipman, D. J. (1990) “Basic local alignment search tool.” J. Mol. Biol. 215:403-410.)

The enzymes claimed may have at least 70%, 75%, 80%, 85%, 90%, 95% or 90% sequence identity to any of the enzymes listed in Table 1. The enzymes may additionally be truncated to the core secondary structure elements to provide function, for example by removing 1 to 20 (for example 1, 2, 3, 4, 5, 6, 7, 8, 9, 10, 11, 12, 13, 14, 15, 16, 17, 18, 19, 20) amino acids from the N and/or C termini of the construct.

Sequence identity is calculated with respect to the full length sequences.

### Isolated

The proteins or nucleic acids may be isolated. By “isolated” means that the protein or nucleotide molecule is not in its native state, insofar as it has been purified at least to some extent or has been synthetically produced, for example by recombinant methods. The term “isolated” therefore includes the possibility of the molecule being in combination with other biological or non-biological material, such as cells, suspensions of cells or cell fragments, proteins, peptides, expression vectors, organic or inorganic solvents, or other materials where appropriate, but excludes the situation where the protein is in a state as found in nature.

### Mutant

By mutant is meant an enzyme which differs from the wild-type, for example the full-length, wild type form (i.e. a variant and these terms are used interchangeably in the application). The mutants may be obtained by various techniques well known in the art. In particular, examples of techniques for altering the DNA sequence encoding the wild-type protein, include, but are not limited to, site-directed mutagenesis, random mutagenesis and synthetic oligonucleotide construction. That is, the variant or mutant may have an altered amino acid sequence.

By corresponding to is meant the equivalent amino acid in any sequence for that enzyme. The corresponding or equivalent amino acid can be found using sequence alignment software such as the BLAST sequence alignment tool described above.

### Nucleic Acids

The nucleic acids may have 70, 75, 80, 85, 90, 95 or 100% sequence identity with those listed in Table 1.

### Expression Cassette

The term “expression cassette” denotes a nucleic acid construct comprising a coding region, i.e. a nucleic acid of the invention, and a regulatory region, i.e. comprising one or more control sequences, operably linked.

The expression cassette may be part of an expression vector. As used herein, the term “expression vector” means a DNA or RNA molecule that comprises an expression cassette of the invention. Preferably, the expression vector is a linear or circular double stranded DNA molecule.

The expression cassette may be part of an in vitro transcription and translation system, or may be expressed by a host cell.

### Host Cell

The host cell may be transformed, transfected or transduced in a transient or stable manner. The expression cassette or vector of the invention is introduced into a host cell so that the cassette or vector is maintained as a chromosomal integrant or as a self-replicating extra-chromosomal vector. The term “host cell” also encompasses any progeny of a parent host cell that is not identical to the parent host cell due to mutations that occur during replication. The host cell may be any cell useful in the production of a variant of the present invention, e.g., a prokaryote or a eukaryote. For example, the prokaryotic host cell may be any Gram-positive or Gram-negative bacterium. The nucleic acid, expression cassette or expression vector according to the invention may be introduced into the host cell by any method known by the skilled person, such as electroporation, conjugation, transduction, competent cell transformation, protoplast transformation, protoplast fusion, biolistic “gene gun” transformation, PEG-mediated transformation, lipid-assisted transformation or transfection, chemically mediated transfection, lithium acetate-mediated transformation, liposome-mediated transformation.

Optionally, more than one copy of a nucleic acid, cassette or vector of the present invention may be inserted into a host cell to increase production of the variant.

In a particular embodiment, the host cell is a recombinant microorganism. The invention indeed allows the engineering of microorganisms with improved capacity to degrade polyester containing material. For instance, the sequence of the invention may be used to complement a wild type strain of a fungus or bacterium already known as able to degrade polyester, in order to improve and/or increase the strain capacity.

### Use

The description of the enzymes above applies to the use of these enzymes.

### Structures of the Enzymes

The atomic co-ordinates of RR1102 (specify variant) have been deposited under accession number PDB ID: 6ZZV.

The atomic co-ordinates of CI5En2-A9S (specify variant) have been deposited under accession number PDB ID: 7NCQ.

The active site residues are given in Tables 2 and 3 below.

### RMSD

The simply root-mean-square deviation (RMSD) is the measure of the average distance between the backbone atoms of superimposed protein structures. The RMSD may be calculated using any algorithm, for example as described in Coutsias E A, Seok C, Dill K A (2004). “Using quaternions to calculate RMSD”. J Comput Chem. 25 (15): 1849-1857.

TABLE 2
Active site residues

| | Active site residues |
|---|---|
| RR1102 | S149/D311/E212/H308 |

TABLE 3
Active site residues for CI5En2

| | Active site residues |
|---|---|
| CI5En2-wt | S138/D184/H216 |

The atomic co-ordinates for these residues can be found in FIGS. 16 and 17. The atomic co-ordinates may be on a computer-readable medium.

The active site residues in addition to Table 3 may include any one or more of the following: Asn 137, Tyr 72, Val 71, Thr 220, Phe 221, Leu 217, Leu 222, Phe 68, Ala 67, Ile 186, Met 139, Ala 187, Trp 163, Leu 66 and Phe 65. For example, the active site residues in addition to those in Table 3 may include all of these residues.

### Uses of the Atomic Co-Ordinates

The atomic co-ordinates may be used to optimise the plastic degrading activity of the enzyme by computational design, i.e. in a computer-implemented method, which may comprise computationally mutating amino acids within the active site to: i) alter the plastic substrate binding or ii) increase the hydrolysis reaction rate. This may be done using https://funclib.weizmann.ac.il/bin/steps. The atomic co-ordinates may also be used in the same way to increase the thermostability of the enzyme. This may be done using https://pross.weizmann.ac.il/step/pross-terms/.

By “optimizing thermostability” is meant increasing the predicted melting temperature of the enzyme.

The atomic co-ordinates may also be used to design a plastic hydrolysing enzyme, for example using the protocol described by Richter F, Leaver-Fay A, Khare S D, Bjelic S, Baker D (2011) De Novo Enzyme Design Using Rosetta3. PLOS ONE 6 (5): e19230.

The atomic co-ordinates used may be the amino acid co-ordinates in FIG. 16 or 17. FIG. 16 lists the amino acid co-ordinates for RR1102. Amino acid residues 12-325 are listed in FIG. 16. FIG. 17 lists the amino acid co-ordinates for CI5En2-A9S. Amino acid residues 2-267 are listed in FIG. 17.

The co-ordinates may additionally be truncated to the core secondary structure elements, for example by removing 1 to 20 (for example 1, 2, 3, 4, 5, 6, 7, 8, 9, 10, 11, 12, 13, 14, 15, 16, 17, 18, 19, 20) amino acids from the N and/or C termini.

Throughout the specification, unless the context demands otherwise, the terms ‘comprise’ or ‘include’, or variations such as ‘comprises’ or ‘comprising’, ‘includes’ or ‘including’ will be understood to imply the method or kit includes a stated integer or group of integers, but not the exclusion of any other integer or group of integers.

### Further Features of the Invention

Additionally, the plastic particle may be alternatively a biopolymer particle. For example, a particle comprising or consisting of a biopolymer such as chitin, lignocellulosic biomass, cellulose, starch, alginate, natural rubbers, cutin, cutan or melanin. By biopolymers is meant polymers that are produced by or derived from living organisms, such as plants and microbes, rather than from petroleum, the traditional source of polymers.

Therefore, the method may be a method for identifying a biopolymer degrading enzyme or optimising a biopolymer degrading enzyme, the method comprising:
a) encapsulating a gene library into a plurality of microfluidic droplets, each microfluidic droplet comprising at least one gene and each microfluidic droplet additionally comprising:
i) an expression system for expressing the gene; and
ii) a biopolymer particle;
b) detecting degradation of the biopolymer particle in the microfluidic droplet; and
c) selecting microfluidic droplets where biopolymer degradation has been detected.

The embodiments described above with regards to the plastic particle apply equally here with respect to biopolymers.

The invention is also described by the following numbered clauses:
1. A method for screening for an optimised polymer degrading enzyme, the method comprising:
a) encapsulating a gene library with variant gene sequences of the polymer degrading enzyme into a plurality of microfluidic droplets, each microfluidic droplet comprising at least one variant gene sequence of the polymer degrading enzyme and each microfluidic droplet additionally comprising:
i) an expression system for expressing the gene; and
ii) a polymer particle;
b) detecting degradation of the polymer particle in the microfluidic droplet; and
c) selecting microfluidic droplets where polymer degradation has been detected.
2. A method for identifying a polymer degrading enzyme, the method comprising:
a) encapsulating a gene library into a plurality of microfluidic droplets, each microfluidic droplet comprising at least one gene and each microfluidic droplet additionally comprising:
i) an expression system for expressing the gene; and
ii) a polymer particle;
b) detecting degradation of the polymer particle in the microfluidic droplet; and
c) selecting microfluidic droplets where polymer degradation has been detected.
3. The method of:
a) clause 1, wherein the gene library is a directed evolution library: or
b) clause 2 wherein the gene library is a metagenomic library; and/or
c) clause 2, wherein the method further comprises making a gene library with variant gene sequences of the identified polymer degrading enzyme and performing the method of clause 1.
4. The method of any of clauses 1-3, wherein the polymer is a biopolymer. For example any of the biopolymers described above.
5. The method of clause 4, wherein detecting degradation is by detecting a reduction in light scattering by the biopolymer particle.
6. The method of clause 2, wherein the method is preceded by a screening method to identify the gene sequence of a polymer degrading enzyme (optionally a biopolymer degrading enzyme), wherein the screening method comprises:
a) encapsulating a gene library into a plurality of microfluidic droplets, each microfluidic droplet comprising at least one gene and each microfluidic droplet additionally comprising:
i) an expression system for expressing the gene; and
ii) a soluble polymer mimic (optionally a soluble biopolymer mimic);
b) detecting cleavage of the soluble polymer mimic in the microfluidic droplet; and
c) selecting microfluidic droplets where cleavage has been detected, optionally wherein the gene library is a metagenomic library.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness. Reference to cited material or information contained in the text should not be understood as a concession that the material or information was part of the common general knowledge or was known in any country

### DESCRIPTION OF THE FIGURES

FIG. 1 shows Functional metagenomics in microfluidic droplets for esterase activity. (a) Pooled metagenomic library from eight different sources contains 1.25×10⁶ members and insert size from 1 to 5 kb. (b) The pooled metagenomic DNA was transformed into high efficient E. coli cells and compartmentalised into water-in-oil droplets under Poisson distribution (λ=0.35, 24.6% of the droplets were singly compartmentalised) together with 20μM of (1) fluorescein dihexanoate substrate and cell lysis reagents. (c) Droplets were incubated off-chip for 24 to 72 hours at room temperature and (d) sorted via FADS. The gate was adjusted to above the fluorescence of the reaction background and the droplets sorted for de-emulsification and (e) DNA recovery. (f) The recovered plasmids were transformed into E. coli BL21 and cells plated on LB agar plates containing 1% (2) tributyrin, which was used for a secondary screen by detecting clear halos around the colony of positive hits. (g) The potential candidates were sequenced and the predicted genes annotated. (h) Genes encoding esterases/lipases or alpha/beta hydrolases were compared to known databases and cloned into expression vector for kinetics and promiscuous profile for the identification of hits with unique features (i).

FIG. 2a. Designs of microfluidic devices used during experiments. (a) Flow-focusing device for the generation of monodisperse water-in-oil droplets. The two aqueous solutions, containing the substrate and the cells were injected from inlet 2 and 3, respectively. Inlet 1 was used for the oil-surfactant mixture. Droplets were collected from outlet 4. The depth of the droplet generation device is 80 μm. The channels at a flow-focusing junction are 50 μm wide and additionally there is a 25 μm-wide constriction facilitating droplet formation. (c) Sorting device for the scatter-activated screening of droplets. Number 5 indicates inlet for the droplet emulsion and numbers 6 and 7 denote the inlets for the spacing and bias oils, respectively. Inlets for the 5 M NaCl solution for generating a high voltage signal are marked by 8 and 9 for ground [GND (−)] and signal [signal (+)] electrodes, respectively. The outlets of the positive (ch+) and negative (ch−) channels are marked by numbers 10 and 11, respectively. Numbers 12 and 13 denote the insertion sites for FC/PC optical fibers, 25 μm, 0.1 NA and 105 μm, 0.22 NA respectively (Thorlabs, UK). The depth of the whole sorting device is 100 μm, with the exception of reinjection chamber which is 50 μm deep. The width of the channel at the sorting junction is 50 μm wide. The scale bar is 5 mm.

FIG. 2b. Scheme of the optical setup used for scatter activated droplet sorting. The setup is a modification of previously presented system for fluorescence activated droplet sorting (van Loo et al. ACS Synth. Biol. 2019, 8, 12, 2690-2700). The large part of microfluidic device was illuminated by the red light coming from the microscope lamp through a 593 nm longpass filter F1 (FF01-593/LP-25, Semrock) and a lens of the microscope condenser (L1). Next, the transmitted red light was collected by the objective (usually 10× or 20×), leaves the microscope through the camera port and it is recorded by a fast camera (Miro eX4, Phantom, USA). The light from the 488 nm laser (Stratus) passed through a neutral density filter OD=1 (NE10A, Thorlabs), and next it was delivered to the sorting junction by coupling via C1 collimator (adjustable FC/PC collimator, CFC-11X-A, Thorlabs) to the incident light optical fiber (25 μm, 0.1 NA, FC/PC, Thorlabs). Usually, the input intensity of the laser was set to 10 mW, which translates to 1 mW on the chip, after passing the ND filter. The light emerging from the detection optical fiber (25 μm, 0.1 NA, FC/PC Thorlabs) was connected via C2 collimator (CFC-11X-A, Thorlabs) to the detector tube housing the F2 bandpass filter (FL488-10 nm, Thorlabs) mounted 50 mm before the detector of photomultiplier tube PMT (PM002, Thorlabs). Simple spherical lenses (L2, L3) were used to focus the light on detectors of camera and PMT. The scattered light signal is recorded by the PMT coupled to a FPGA NI card and analysed by a custom written LabVIEW program in real time. Black arrows indicate the direction of signals and triggers in the system. When a scatter light signal was higher than an arbitrarily set threshold, the FPGA card triggered the generation of a high voltage pulse (1 kV) by the series of electronic devices: pulse generator PG (TGP110, Thurlby Thandar Instruments)->function generator FG (TG200, Thurlby Thandar Instruments)->high voltage amplifier AMP (610E, Trek). A short 2-ms-long pulse was delivered with a delay of 2.5 ms to the microfluidic device by ‘salt electrodes’ filled with 5M NaCl solution7 and, as a result, highly fluorescent droplets were derailed to the collection channel for positive ‘hits’ (b). The duration and delay of pulse can be modified according to the flow rates and the desired throughput of the sorting (up to 350 Hz), depending on the quality of input emulsion.

FIG. 3 shows From fluorescence droplet sorting to identification of positive hits. A metagenomic library of above 1 million members was sorted for esterase activity by using (1) fluorescein dihexanoate as a bait substrate. The water-in-oil droplets were incubated up to 3 days at room temperature and sorted above the threshold via FADS, being the fluorescence histograms of the droplets sorted after 1 day (10 millions of screened droplets, 126 of sorted droplets-representing a sorted fraction of 0.0012%) and 2 days (5.7 millions of screened droplets, 110 of sorted droplets-representing a sorted fraction of 0.0019%) incubation of campaign I are shown here (A). After droplet sorting and DNA recovery, the recovered plasmids were transformed into cells for secondary screen (B and C). (B) Through the hydrolysis of 1% (2) tributyrin to glycerol and butanoic acid, clear halos are formatted around the colonies of potentially positive candidates for esterase activity. (C) The activity was further confirmed via reaction with fluorescein dihexanoate. The hydrolysis of fluorescein dihexanoate to fluorescein monohexanoate and hexanoic acid by the positive candidates can be monitored when using a fluorescence plate reader. P35 and Est30 were used as a negative and positive control, respectively, being the dotted line in grey representing the background reaction of negative control. The reaction was performed in triplicate and the standard error of the measurements are shown as error bars.

FIG. 4 shows Metagenomic hits: exploration of alpha/beta hydrolase superfamily and its activity. The sequence context of the metagenomic hits were investigated together with their functional activity against p-NP acyl esters, pH profile and melting temperature. (A) A sequence network was generated with sequences retrieved from alpha/beta hydrolase clan (CL0028 from Pfam classification) and the 13 metagenomic hits identified in this work. The network created contains 370,000 sequences clustered into 20,000 nodes representing 80% of CL0028. The node size is proportional to the number of sequences represented (range 1 to 2600, median 4). Connecting lines represent alignments with e-values <10⁻²⁰, the median alignment length was 312. (B, C and D) Functional characterisation of metagenomic hits. The ORFs encoding for esterase/lipase activity were individually cloned into an expression vector and the corresponding recombinant proteins were tested against p-NP acyl esters (B), pH (C) and melting temperature (D). (B) Catalytic efficiencies of the metagenomic hits were measured against different p-NP acyl esters [acetate (C2), butyrate (C4), hexanoate (C6), octanoate (C8), decanoate (C10), dodecanoate (C12), myristate (C14) and palmitate (C16)]. The reaction was performed in buffer containing 50 mM Tris-HCl pH 8.0 and 0.3% (v/v) Triton X-100 at 22° C. (C) pH profile was performed against 1 mM p-NP C6 in buffer citrate (at pH 4.0, 5.0 and 6.0), phosphate (at pH 6.0, 7.0 and 8.0), Tris-HCl (at pH 7.0, 8.0 and 9.0) or CAPS (at pH 9.5, 10.0 and 11.0). (D) The melting temperature (Tm) was determined via thermal shift assay, where their denaturation promote the release of Sypro orange and the fluorescence (λ_{excitation}=410 nm, λₑₘᵢₛₛᵢₒₙ=610 nm) released was measured through a ramp of temperature from 20 to 100° C. The experiment was performed in triplicate and the data was normalized before analysis on Kaleidagraph. The assay was performed in buffer containing 100 mM Tris-HCl pH8.0 and 100 mM NaCl.

FIG. 5 shows Exploring the sequence space for PET hydrolase discovery. (A) Sequence network of currently known PETases and a novel esterase metagenomic-derived, RR11O2. 3408 sequences were retrieved from MGnify and NCBI databases, and the obtained sequence network is shown here (sequences from 20% to 50% identity are not displayed). Grey nodes represent sequences extracted from MGnify and NCBI. Red node represents RR11O2, blue nodes represent known PETases, green and orange nodes represent reported cutinases and esterases showing PET activity, respectively, and light blue nodes represent described polyester degrading enzymes in the literature. To putatively characterise sequence clusters, the identification of PETases, cutinases, esterases and RR11O2 in the network helped to define the potential clusters. The cluster of RR11O2, CI5En2 and PETase are written in grey. (B) PET hydrolases catalyze the depolymerization of poly(ethylene terephthalate) (PET) to bis(2-hydroxyethyl) terephthalate (BHET), mono(2-hydroxyethyl)-terephthalic (MHET) or terephthalic acid (TPA) and ethylene glycol (EG). During characterizations via high-performance liquid chromatography (HPLC) MHET and TPA have been identified as main products. (C) Enzymatic degradation of PET by RR11O2, CI5En2 and controls LCC, LCC-ICCG and IsPETase. HPLC analysis of the compounds released from the crystalline PET film after three days of incubation at 37° C. using 6 μM of the enzymes. Absorbance at 250 nm was monitored.

FIG. 6 shows Functional screening of enzymes from metagenomic libraries via fluorescence-activated droplet sorting (FADS) followed by the cycle of directed evolution based on scatter light activated sorting of microdroplets (SADS). (A) As described in FIG. 1, for the screening of metagenomic libraries for esterase activity isolated environmental DNA (eDNA) was cloned into plasmids. After the transformation of bacterial cells, single ones were co-encapsulated in microdroplets with lysis agent and the fluorescein dihexanoate substrate. After an off-chip incubation, the droplets were reinjected into the FADS sorting device and positive hits selected. After DNA recovery the re-screening and sequencing lead to a novel esterase showing promiscuous activity on PET. This enzyme was used to generate a sequence network together with known PETases. CI5En2 was identified in of the clusters as a novel PET hydrolase. This enzyme was subjected to a directed evolution campaign. Single bacterial cells were encapsulated in microdroplets together with lysis agent and PET nanoparticles serving as the substrate of the assay. Droplets were incubated in droplet chambers and reinjected into the sorting module. The SADS module relies on fiber optics integrated into a chip at a 90° angle. Schematic picture showing the main principle of the assay. If an enzyme degrades the plastic nanoparticles, the intensity of scattered light is reduced. The DNA was recovered from positive droplets and subjected to re-screening and sequencing resulting in improved PET hydrolases. For further improvement iterative rounds of evolution were performed. (B) Subsequent individual video frames depicting a one sorting event. The microdroplet sorting device measures the intensity of light scattering as droplets flow in front of the fibers. The dielectrophoretic sorting of “positive” droplets is triggered when the measured value is within a user-defined threshold. (C) Histogram of the detected light scattering for co-encapsulated CI5En2 (600 nM) or a buffer control and PET nanoparticles. The signal for droplets containing the enzyme decreases over time due to depolymerisation of PET. (D) Histogram showing the results of the sorting experiment with the single cell droplet lysates expressing error-prone PCR library of CI5En2. The sorting was performed after two weeks of incubation at RT. The values of light scattering from sorted positive droplets are marked by green colour and represent 0.5% of the population.

FIG. 7 shows The structural features of RR11O2, a member from protein of unknown function (DUF3089). (A) The protein structure of RR11O2 with chain A (left side) displayed as B-factor analysis and chain B (right side) in cartoon (deep salmon color for a-helices and black for B-strands). (B) Detail of RR11O2's cavity (in blue) and the catalytic triad (in cyan) S149, D311 and H308. (C) The catalytic efficiencies of RR11O2 measured against p-NP acyl esters [acetate (C2), butyrate (C4), hexanoate (C6), octanoate (C8), decanoate (C10), dodecanoate (C12), myristate (C14) and palmitate (C16)]. The reactions were performed in buffer containing 50 mM Tris-HCl PH 8.0 and 0.3% (v/v) Triton X-100 at 22° C.

FIG. 8 shows The predicted binding modes of para-nitrophenol acyl esters with different chain lengths. The largest binding site of RR11O2 predicted by the PRANK server (18) that is believed to be the active site is colored in red (pocket A) and blue (pocket B). (A) All 10 poses of the binding modes generated for each substrate binding to the active site of RR11O2. Substrates with alkyl chains C2 to C8 are predicted to bind within the inner active site pocket (pocket A) in all the predicted binding modes. Whereas C10 to C16 are predicted not to fit within the active site pocket (pocket A). This predicted binding mode is validated by the lack of activity for substrates with chain length C10 to C16. (B) The activities of the substrates used in the modelling studies. (C) The structures of the substrates used in the modelling studies.

FIG. 9a shows Screening and characterization of improved variants. (A) Enrichment of variants with higher activity compared to wildtype after sorting the ep-PCR library of CI5En2. After DNA recovery and transformation, colonies were randomly picked from the unsorted original library and the sorted samples after 8 days, two and five weeks, followed by a plate-assay against the p-NP C12 substrate. The data were normalized to the rate of CI5En2-wt. The bar graphs give a comparison of variants showing higher activity (activity>wt) or lower activity (activity<wt) compared to wt. (B) Enrichment of variants with higher activity than wildtype after three rounds of evolution of CI5En2. Box plots show mean (°), median (-), quartiles (boxes), and range (whiskers). (C) Activity of improved variants in comparison to CI5En2-wt and the metagenomic-derived hit RR11O2. PET nanoparticles were incubated with 6 μM enzyme for one hour at 37° C. Amount of the released monomers TPA and MHET was determined via HPLC and corresponding standard curves for TPA and MHET. (D) Results of the time-course experiment performed over 22 hours in microdroplets incubating amorphous PET NPs with 600 nM A10 and CI5En2-wt are shown.

FIG. 9b shows Thermostability of Bla-CI5En2-wt, its variants and IsPETase. The purified enzymes had their melting temperature (Tm) determined via thermal shift assay, where their denaturation promote the release of Sypro orange and the fluorescence (λ_{excitation}=410 nm, λₑₘᵢₛₛᵢₒₙ=610 nm) released is measured through a ramp of temperature from 25 to 95° C. The experiment was performed in triplicate and the data was normalized before analysis on Kaleidagraph. The assay was performed in buffer containing 100 mM Tris-HCl pH8.0 and 100 mM NaCl.

FIG. 10 shows Structural features of CI5En2-A9S (PDB code 7NCQ). (A) Protein structure of CI5En2-A9S obtained at high resolution of 2.4 Å. CI5En2-A9S displays α/β folding and two molecules were observed in the unit cell (chain A and B). (B) Residues composing the catalytic triad (S138, D184 and H216).

FIG. 11 shows RR11O2 has a clear entrance for substrates and an internal cavity. (A and B) RR11O2 is displayed as blue surface and the entrance which gives access to the catalytic triad is highlighted with circular dots and arrows in grey (A) and black (B and C). B—The surface of the protein is illustrated with 60% transparency allowing the visualization of the residues of the catalytic triad (S149, D311 and H308) inside of the protein. (C) The cavity of RR11O2 is illustrated at the same position as in (A) and (B), and also after 90° turn to the right side. The position of entrance is shown with the black arrow.

FIG. 12 shows Secondary and third structure comparison of RR11O2 and a member from α/β fold family. (A) Map of the secondary structure of RR11O2. The colours of structurally conserved secondary structure between RR11O2 and other α/β fold hydrolayses are matched. (B) General map of the secondary structure of α/β hydrolayse. The colours of structurally conserved secondary structure between RR11O2 and other α/β fold hydrolayses are matched. Grey secondary structure is not conserved; (C) The β-sheets and a-helixes of the α/β hydrolayse fold that is conserved in RR11O2; For (A), (B) and (C), the colours of structurally conserved structures are matched. (D) The structural alignment of RR11O2 (PDB code 6ZZV) against a representative α/β hydrolayse fold (PDB code 1UFO). The alignment was performed in Pymol using the super function; (E) The structural alignment of the B-sheets of RR11O2 with the B-sheets of PDB code 1UFO; (F) The structural alignment of the conserved α-helixes of RR11O2 and 1UFO; The structural alignment of the conserved α-helixes and β-sheets of RR11O2 and 1UFO; (G) The full structural alignment of RR11O2 (PDB 6ZZV) against the full structure of a representative of α/β hydrolase fold (PDB code 1UFO).

FIG. 13 shows The catalytic triad of RR11O2's (PDB 6ZZV). The structural alignment of the conserved α-helix α-F from RR11O2 and α-C from PDB code 1UFO. (A) The position of nucleophilic (Ser149 for RR11O2 and Ser113 for PDB code 1UFO), (B) The position of Asp183 from the catalytic triad of PDB code 1UFO compared to the position of Asp311 and Glu212 in RR11O2. There is very little conservation of secondary structure in this section of RR11O2; (C) The structural alignment of α-J from RR11O2 and α-E from PDB code 1UFO. The position of the basic His from the catalytic triad of both structures is shown; (D) The position of the catalytic triad in the 10 structures with the highest structural similarity identified by the DALI server; (E) The position of possible third members of the catalytic triad in the active site of RR11O2.

FIG. 14 shows an Investigation of loop flexibility on RR11O2's structure (PDB 6ZZY). (A) The normalized B-factors for chain A of RR11O2′s 3D structure for the two active site loops, loop 1 and loop 2. (B) The position of the flexible active site and loop (loop 1) in the structure of RR11O2. (C) The structure of chain A of RR11O2 where the colour and size represents the B-factor; (D) The position of the flexible active site loop and the predicted binding mode of the substrate.

FIG. 15a shows Hydrodynamic diameter distribution of PET NPs. Particle sizes were determined by dynamic light scattering (DLS) starting from amorphous film or crystalline powder. Polydispersity index (PdI) of the suspension is shown.

FIG. 15b shows Enzymatic degradation of amorphous and crystalline PET NPs. HPLC analysis of the products released after one week incubation at 30° C. using 6 μM pExp-bla-CI5En2-WT. Absorbance at 250 nm was monitored which detects released TPA and MHET.

FIG. 15c shows Dynamic Light Scattering (DLS) measurements of stabilized nylon 6 and nylon 66 nanoparticles (NPs) to determine size distribution. (A) nylon 6 NPs stabilized with 0.1% SDS. (B) nylon 66 NPs stabilized with 0.1% SDS. d: diameter; Pdl: polydispersity index.

FIG. 16 shows Atomic co-ordinates of RR11O2.

FIG. 17 shows Atomic co-ordinates of CI5En2-A9S soaked with BHET.

FIG. 18 shows the Protein structure of CI5En2-A9S soaked with BHET (PDB code 7ZJ9). Crystals of CI5En2-A9S were obtained under condition Proplex H2 (0.1 M MES pH 6.5, 1 M LiSO4) and they were soaked in mother reservoir containing 3.5 mM for 46 min. A high resolution dataset was obtained at 2.1 Å. Surface (A) and cartoon (B) display of CI5En2-A9S in complex with a product of BHET hydrolysis, TPA. (C) Catalytic triad of CI5En2 (S138, D184 and H216, in green) in complex with TPA (in blue). The presence of sulfate from the crystallization condition is illustrated in all figures.

FIG. 19 shows the Protein structure of CI5En2-A9S co-crystallized with polycaprolactone, PCL (PDB code 7ZJA). CI5En2-A9S was crystallized in the presence of PCL nanoparticles and crystals were successfully formed under the conditions of ‘Classics H8’ [0.1 M MES pH 6.5, 30% w/v, PEG MME 5K, 0.2 M (NH₄)₂SO₄]. A high resolution dataset at 1.9 Å of CI5En2-A9S co-crystallized with PCL was obtained. Surface (A) and cartoon display (B) of CI5En2-A9S in complex with a product of PCL hydrolysis, 6-hydroxycaproic acid. (C) Catalytic triad of CI5En2 (S138, D184 and H216, in green) in complex with 6-hydroxycaproic acid (in blue).

FIG. 20 shows a) Dynamic Light Scattering (DLS) measurements of 5 mg/mL lignin nanoparticles (NPs) to determine size distribution (d: diameter; Pdl: polydispersity index) and b) Scatter-activated measurements of droplets containing lignin nanoparticles (5 mg/mL lignin nanoparticles were encapsulated in 150 pL droplets).

FIG. 21 shows Sequence space exploration for the discovery of novel nylonases. (A) Homologues of NylC (NCBI access code: Q1EPR5.2) were extracted from MGnify database and used to generate a sequence network composed of 1583 nodes and 69291 edges sequences. (B) The first neighbours of NylC were identified and the closest candidates were selected (C), among them Nyl2 (MGnify code: MGYP000645618208).

FIG. 22 shows Sequence space exploration for the discovery of novel nylonases. (A) Homologues of NylC (NCBI access code: Q1EPR5.2) were extracted from MGnify database and used to generate a sequence network composed of 1583 nodes and 69291 edges sequences. (B) The first connections of MGYP000645618208 (sequence retrieved from MGnify under the listed code) were identified and the sequence space divided into clusters (from 1 to 3). Potential nylonase candidates were selected from each cluster, including: Nyl5 (MGnify code: MGYP000544655629, from cluster 1) and Nyl9 (MGnify code: MGYP000403660814, from cluster 3).

FIG. 23 shows High-performance liquid chromatography (HPLC) analysis of the products released from the nylon 6 nanoparticle depolymerisation. The reaction was monitored over one week of incubation at 37° C. using 10 μM of the enzymes Nyl-2, Nyl-3, Nyl-5, Nyl-9 and the buffer control. Absorbance at 220 nm was measured which detects released 6-aminohexanoic acid. The time offset is 2%.

FIG. 24a shows High-performance liquid chromatography (HPLC) analysis of the products released from the nylon 6 nanoparticle depolymerisation. The reaction was measured after an incubation at 37° C. after 3 days using 18 μM of the enzyme Bla-NylSoil and the buffer control. Absorbance at 220 nm was measured which detects released 6-aminohexanoic acid. FIG. 24b shows High-performance liquid chromatography (HPLC) analysis of the products released from tights sample depolymerisation. The reaction was measured over a time period of 1 week and incubation at 37° C. using 18 μM of the enzymes Nyl-2, Nyl-5, Nyl-9, Nyl-10, Nyl-11, Nyl-12, Nyl-14, Nyl-Mar-2, Nyl-18, Nyl-20, Nyl-24, Nyl-25 and the negative buffer control and positive Nyl-C control. Absorbance at 220 nm was measured which detects released 6-aminohexanoic acid (Ahx).

FIG. 25 shows Sequence space exploration for the discovery of novel nylonases on Uniprot/SwissProt/NCBI databases. Homologues of Amidadase (NCBI code WP_011209364.1), NylA (NCBI access codes: P13397 and P13398), NylB (NCBI access code P07061) and NylC (NCBI access code: Q1EPR5.2) were extracted from Uniprot/SwissProt/NCBI databases and used to generate a sequence network composed of 9,770 sequences. The first neighbours of NylC were identified and 295 sequences retrieved for phylogenetic analysis. Among the sequences retrieved are Nyl-10 (Uniprot code A0A1C6V3K0), Nyl-11 (Uniprot code A0A138ZXG5), Nyl-12 (Uniprot code A0A1M7HAK8) and Nyl-14 (Uniprot code WP243569406.1).

FIG. 26 shows Sequence space exploration for the discovery of novel nylonases on MGnify, full-length database. (A) Homologues of Amidadase (NCBI code WP_011209364.1), NylA (NCBI access codes: P13397 and P13398), NylB (NCBI access code P07061) and NylC (NCBI access code: Q1EPR5.2) were extracted from MGnify/full length database and used to generate a sequence network composed of 10,233 sequences. (B) The first neighbours of NylC were identified and (C) 497 sequences retrieved for phylogenetic analysis. Among the sequences retrieved are Nyl 18-FL (MGnify code MGYP000370831902/2-317), 20-FL (MGnify code MGYP000123679597/3-313), 24-FL (MGnify code MGYP000614730541/11-353) and 25-FL (MGYP000554001943/18-373).

FIG. 27 shows Sequence space exploration for the discovery of novel nylonases on MGnify, soil database. Homologues of Amidadase (NCBI code WP_011209364.1), NylA (NCBI access codes: P13397 and P13398), NylB (NCBI access code P07061) and NylC (NCBI access code: Q1EPR5.2) were extracted from MGnify/soil database and used to generate a sequence network composed of 1,108 sequences. The first neighbours of NylC were identified and 77 sequences retrieved for phylogenetic analysis. Among the sequences retrieved is Soil 1 (MGnify code MGYP000120183263/1-286).

FIG. 28 shows Sequence space exploration for the discovery of novel nylonases on MGnify, marine database. (A) Homologues of Amidadase (NCBI code WP_011209364.1), NylA (NCBI access codes: P13397 and P13398), NylB (NCBI access code P07061) and NylC (NCBI access code: Q1EPR5.2) were extracted from MGnify/marine database and used to generate a sequence network composed of 7,089 sequences. The first neighbours of NylC were identified and (C) 344 sequences retrieved for phylogenetic analysis. Among the sequences retrieved is Marine 2 (MGnify code MGYP000645432643/3-305).

### EXAMPLES

Aspects of the present invention will now be illustrated by way of example only and with reference to the following experimentation.

### General Materials and Methods

### Fabrication of Microfluidic Chips for Scattering Activated Droplet Sorting (SADS)

The microfluidic devices used for droplet generation and droplet sorting were fabricated following classical soft lithography procedures by using high-resolution acetate masks and SU-8 photoresist patterning. In order to make a single PDMS [poly(dimethyl siloxane)] microfluidic device, 20-30 grams of silicone elastomer base and the curing agent (Sylgard™ 184, Dow Corning, USA) were mixed in the a 10:1 (v:v) ratio in a plastic cup and next degassed in a vacuum chamber. Next, PDMS was poured on SU-8 master wafer and cured in the oven at 65° C. for at least 4 h. The patterned PDMS chip was then plasma bonded to a 50 mm×75 mm×1 mm (length×width×thickness) glass slide in a low-pressure oxygen plasma generator (Femto, Diener Electronics, Germany). Next, the device was flushed with 1% (v/v) trichloro (1H,1H,2H,2H-perfluorooctyl)-silane (Sigma Aldrich, UK) in HFE-7500 (3M, USA) and baked on a hot plate at 75° C. for 30 minutes in order to evaporate the fluorocarbon oil and silane mix.

The fabrication of the sorting chip required additional integration with incident light and detection multimode optical fibers with FC/PC connector type. The incident light fiber (cat. no M67L02, Thorlabs) had a cladding diameter of 125 μm and a core diameter of 25 μm with numerical aperture (NA) of 0.1. The detection fiber (cat. no M43L02, Thorlabs) had a cladding diameter of 125 μm and a core diameter of 105 μm with NA of 0.22. Both fibers were cut at their ends to remove one of the FC/PC connecters, next outer protective PVC jacked was removed using a three-hole fiber stripper (cat. no FTS4, Thorlabs), Kevlar® protective threads were cut with a scalpel and finally acrylate coating was using a fiber stripping tool (cat. no T06S13, Thorlabs). In the next step the tip of the fiber tip was cleaved using a ceramic fiber scribe (cat. no CSW12-5, Thorlabs) in order to obtain flat tip end. The quality of the cleavage was inspected by passing a low power (e.g. 0.1 mW) laser light through the fiber and a visual inspection of the shape of a beam emerging from the fiber tip end. If necessary, the cleavage was repeated until spherical shape of the light beam was observed. The fiber ends were fixed to the microfluidic chip at least 1 day prior to an experiment. The fiber fixing process was performed on the microscope stage and a microscope camera was used to verify the position of the fiber ends. First, the microfluidic channels housing the fibers were filled with liquid PDMS and then fiber tips were manually inserted to the chip. Fibers were additionally stabilized by attaching them to the glass slide of the chip with epoxy glue (Araldite® Two Component Epoxy Paste Adhesive). The whole chip was left overnight on the microscope stage to let the PDMS and glue cure at room temperature. Such chips can be used for several sorting experiments, provided that they are washed carefully with pure HFE-7500 and dried by blowing with compressed air over them after each use.

### Expression of the Gene Library

To express the enzyme, the following protocol may be used:
Transform E. coli cells with gene library and grow the cells in a culture flask.
When cells reach a specific OD, induce the protein expression using IPTG. For example, induce with 0.4 mM IPTG at an OD of 0.5.
Incubate the cultures overnight at 20° C.
Encapsulate single cells into droplets together with lysis agent.

Another strategy that may be used is to encapsulate single cells into droplets together with culture media. First grow cells within a droplet, then picoinject IPTG to induce protein expression. Afterwards picoinject lysis agent and plastic nanoparticles.

### Preparation of PET Films

PET films used for enzymatic degradation were made by dissolving 200 mg of PET granules in 10 mL dichloromethane or a 4:1 mixture of chloroform and trifluoroacetic acid, respectively. 500 μL or 1 mL of the dissolved solution were evaporated in an open 1.5 ml tube at 96° C., respectively. Crystallinity of the plastic film was determined using Differential Scanning calorimetry (DSC) (1.5.).

### Characterization of Plastic Substrates by Differential Scanning calorimetry (DSC)

PET samples (granulate, films and nanoparticles) were analyzed by DSC Q2000 instrument (TA instruments, New Castle, USA) equipped with an autosampler and an intercooler system.

Dry samples of approximately 1 mg were subjected to a temperature program composed of a first heating, cooling, and a second heating cycle under a nitrogen atmosphere. The first heating cycle between −20 and +300° C. was run at a rate of 5 K/min. Subsequent cooling to 0° C. and a second heating cycle to 300° C. were performed at a constant rate of −10 K/min and 10 K/min, respectively. The resulting thermograms were analyzed using TA Universal Analysis 2000 software. From the first heating cycle, the glass transition temperature T_{g}, the melting temperature Tₘ and the heats of melting ΔHₘ and of cold crystallization ΔH_{c} were calculated. Percentage of crystallinity K was calculated by the equation below:

$$K = 100 \times \frac{Δ {H}_{m} - {H}_{c}}{{H}_{m} 100 \%}$$

ΔHₘ is the enthalpy of melting that can be determined by integrating the endothermic melting peak, ΔH_{c} is the enthalpy of cold crystallization and can be determined by integrating the exothermic cold crystallization peak. The experimental heat of fusion for the polymer is divided by the literature value reported for the enthalpy of melting a 100% crystalline polymer, which is 140 J/g for PET.

### DNA Sequencing, Primer Walking and ORF Analysis

All the colonies that showed a clear halo during the secondary screening were selected for Sanger DNA sequencing using the M13-Forward (5′-TGT AAA ACG ACG GCC AGT-3′) and M13-Reverse (5′-CAG GAA ACA GCT ATG AC-3′) primers. Contigs were generated using Sequencher version 5.4.5 (Genes Codes Corporation). If needed, primer walking sequencing method was performed. All the obtained contigs were compared with the sequences deposited in the National Center for Biotechnology Information (NCBI), through the local alignment BLAST tool against non-redundant protein (nr) and metagenomics (env_nr), and Pfam databases. ORFfinder (https://www.ncbi.nlm.nih.gov/orffinder/) was used to perform ORF prediction by using any alternative initiation codon and the predicted ORFs for esterase/lipase activity were compared towards Pfam, ESTHER and UniProtKB databases.

### Phylogenetic Analysis

The amino acid sequences of 48 members representing the 19 families described in the literature were retrieved from public databases. These sequences together with the 13 sequences of the metagenomic-derived hits identified in this work were used as input file for a sequence alignment using ClustalW and the output file generated was submitted to MEGA 7 to build a suitable evolutionary model. The phylogenetic tree was built by using the Maximum Likelihood method base on the JTT matrix-based model with a bootstrap of1,000 replicates.

### Cloning of Selected

All the potential ORFs for esterase activity were selected for cloning for further biochemical characterisation. The selected genes were amplified by PCR with Phusion High-Fidelity DNA Polymerase (catalogue number F530L, ThermoFisher Scientific) using designed primers with sites for the restriction enzymes. The amplified fragments were cloned into pHAT or pHAT3vectors for protein expression and the recombinant plasmids were used for transformation of E. coli DH5a, E. coli BL21 (DE3) (catalogue number C2527H, New England BioLabs) or SHuffle T7 Express (catalogue number C3029H, New England BioLabs) cells.

### Protein Overexpression and Purification

The transformed cells were grown at 37° C. for 16 hours in 5 mL of LB media containing 100 μg mL-1 ampicillin and obtained cultures were used to inoculate 500 ml of LB (100 μg mL-1 ampicillin). Cells were grown at 37° C. until the OD₆₀₀ₙₘ reached 0.5 to 0.6 and the protein expression was induced by adding 400 μM of IPTG at 20° C. for 20 hours. After expression, cells were harvesting by centrifugation at 4000 g for 20 minutes at 4° C. and resuspended in 35 mL of 50 mM Tris-HCl pH 8.0 for cell lysis via Emulsiflex (Avestin). The extract was brought to 50 mM Tris-HCl PH 8.0, 250 mM NaCl, 10 mM imidazole, 10 mM B-mercaptoethanol, and 5% (v/v) glycerol and centrifuged at 8,000 g for 45 min at 4° C. to remove cell debris. The supernatant was loaded onto Super Nickel NTA Affinity Resin (catalogue number Super-NiNTA25, Protein Ark) which was pre-equilibrated with purification buffer [50 mM Tris-HCl pH 8.0, 250 mM NaCl, 10 mM imidazole and 5% (v/v) glycerol] and the recombinant proteins were eluted using a stepwise imidazole gradient (10, 50, 100, 200, 500 mM imidazole). The soluble expression of the recombinant proteins was confirmed by SDS-PAGE gel and the selected fractions were concentrated by centrifugation with Amicon Ultra-15 filters (Merck-Millipore) using the appropriate cut-off based on their molecular weight. In order to eliminate contaminants an additional protein purification step was performed by size-exclusion chromatography using the columns HiLoad 16/60 Superdex 75 or 200 (GE Healthcare) at 4° C. For the samples without contaminants, the buffer exchange was performed by Sephadex G-25 PD10 column (catalogue number 17085101, GE Healthcare) according to manufacturer's protocol. All the recombinant proteins were stored in 50 mM Tris-HCl PH 8.0, 100 mM NaCl and 2.5% (v/v) glycerol and the protein concentration was determined by measuring absorbance at 280 nm using Nanodrop ND-1000 spectrophotometer (Nanodrop Technologies).

### Substrates Used in This Work Include

(1) fluorescein dihexanoate, (2) tributyrin, (3c) pNP-hexanoate; (3e) pNP-decanoate; (3f) pNP-dodecanoate.

### Biochemical Characterisation

The activity of all esterase hits was determined via hydrolysis of p-nitrophenol (p-NP) carboxylesterase by monitoring the release of p-nitrophenol. The amount of p-nitrophenol released was quantified by absorbance at 405 nm (εpNP=17,000 M−1.cm−1) using a Spectramax 190 microplate reader (Molecular Devices). Unless otherwise indicated, the measurements were performed at 22° C. in triplicate using 96-well plates with a final assay volume of 100 μL in 50 mM Tris-HCl pH 8.0 and 0.3% (v/v) triton X-100. The substrate specificity was investigated by measuring the hydrolysis of the following p-NP-fatty acyl esters: butyrate (C4), hexanoate (C6 from TCI Chemicals, H0484), octanoate (C8), decanoate (C10), dodecanoate (C12), myristate (C14) and palmitate (C16). The kinetic parameters for each substrate were obtained by non-linear regression of the data using the Michaelis-Menten equation: V=Vmax*[S]/(Km+[S]), where V is the rate, Vmax is the maximum rate achieved by the reaction, S is the substrate concentration, and Km is the Michaelis-Menten constant. The effect of the pH was evaluated by measuring the enzyme activity at pH 4.0 to 11.0 in 50 mM of the following buffers containing 0.3% (v/v) triton X-100: sodium citrate at pH 4.0, pH 5.0 and 6.0; phosphate at pH 6.0, 7.0, 8.0; Tris-HCl at pH 7.0, 8.0 and 9.0; N-cyclohexyl-3-aminopropanesulfonic acid (CAPS) at pH 9.5, 10.0, 11.0.

### Assay of Thermostability

The melting temperature curves were recorded on a Rotor-Gene 6000 Real Time PCR Machine (Corbett), where the fluorescence was excited at 470 nm and measured at 610 nm between 25 and 95° C. in steps of 0.5° C. The samples were prepared in triplicate and contained 8 μM of enzyme in the 50 mM Tris-HCl PH 8.0, 100 mM NaCl, 2.5% glycerol and 5×Sypro Orange Protein Gel Stain (ThermoFisher). The samples were prepared for a final assay volume of 20 μL, which was adjusted with a buffer containing 100 mM Tris-HCl PH 8.0 and 100 mM NaCl. The melting curves were analysed on KaleidaGraph (Synergy Software) and fitted by using the equation m1+((m1−m2)/(1+exp((m3−M0)/m4))).

### Circular Dichroism (CD)

The CD measurements were carried out with a JASCO J-810 spectropolarimeter equipped with a Peltier-type temperature controller and a thermostatic cell holder, which was interfaced with a thermostatic bath. To verify the influence of pH in RR11O2 and RR11O2 M1_L9del folding, the enzymes were diluted in the buffers: 100 mM sodium phosphate at pH 6.0, 7.0 and 8.0, and in 50 mM borate at pH 8.8. Three consecutive scans from 190 to 250 nm of each experiment were compiled and the average spectra subtracted for the corresponding buffer baseline contribution. The measurements were performed at the concentration of 5.2 μmol and 5.39 μmol of RR11O2 and RR11O2 M1_L9del, respectively.

### Cloning, Expression and Purification of IsPETase

The IsPETase gene (GenBank accession number, GAP38373.1) was inserted into pCri18-a (Addgene, #61326) plasmid by Gibson-Assembly-PCR6 generating plasmid pCri18-a-IsPETase. B. subtilis 168 was transformed with this plasmid. The recombinant protein contains a C-terminal 6×His tag for purification. The recombinant B. subtilis strain was grown in 2YT medium supplemented with 5 μg/mL chloramphenicol at 37° C. After induction by adding 0.5 mM IPTG, the culture was further incubated at 20° C. for 18 h. The culture was centrifuged (13,000×g, 10 min, 4° C.) and supernatant applied to Ni-NTA column equilibrated with buffer A containing 50 mM Tris-HCl, 150 mM NaCl and 50% glycerol (pH 8). After washing with 20column volumes of buffer A containing 20 mM imidazole, the bound proteins were eluted with 500 mM imidazole in buffer A. The enzyme in the elution buffer was further changed to buffer A by an Amicon ultra-4 centrifugal filter device (Millipore, USA) and then stored at 4° C. The degree of protein purity was confirmed by sodium dodecyl sulfate polyacrylamide gel electrophoresis and the concentration determined by Nanodrop 2000 (Thermo Fisher Scientific, USA) using Mw of 30.2 kDa and extinction coefficient ε=39,400 M−1 cm−1.

### Example 1: Initial Pre-Screening Method

To screen for esterase activity at high sensitivity and throughput, we have developed a screening platform using microfluidics. To explore the diversity provided by metagenomics, a previously generated DNA metagenomic library derived from eight different sources (marine sludge, goose pond, sand soil, vanilla pods, medium compost, thermophilic ripe compost and cow rumen) and composed of 1,250,000 members was encapsulated into water-in-oil droplets taking into account the insights gained from the substrate and buffer screening and the enrichment achieved for the active esterase Est30.

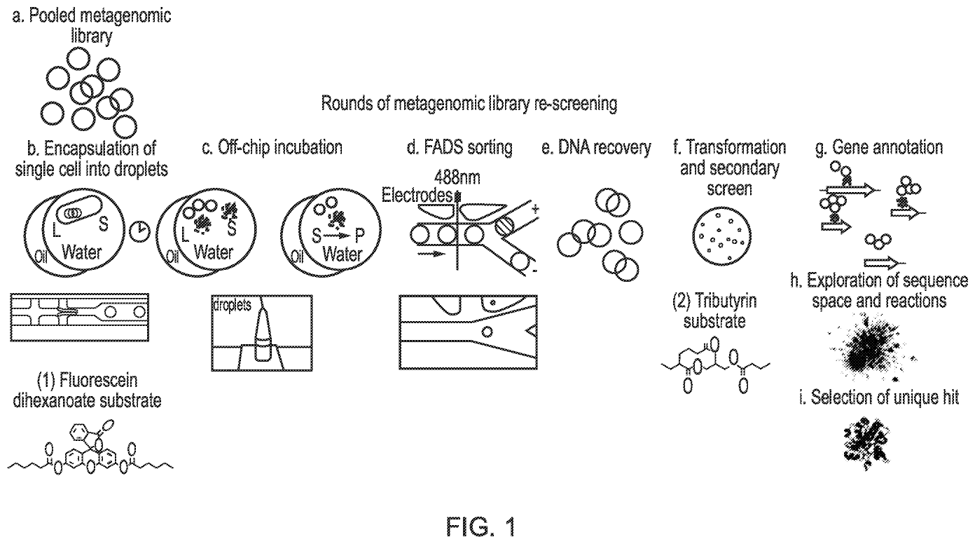

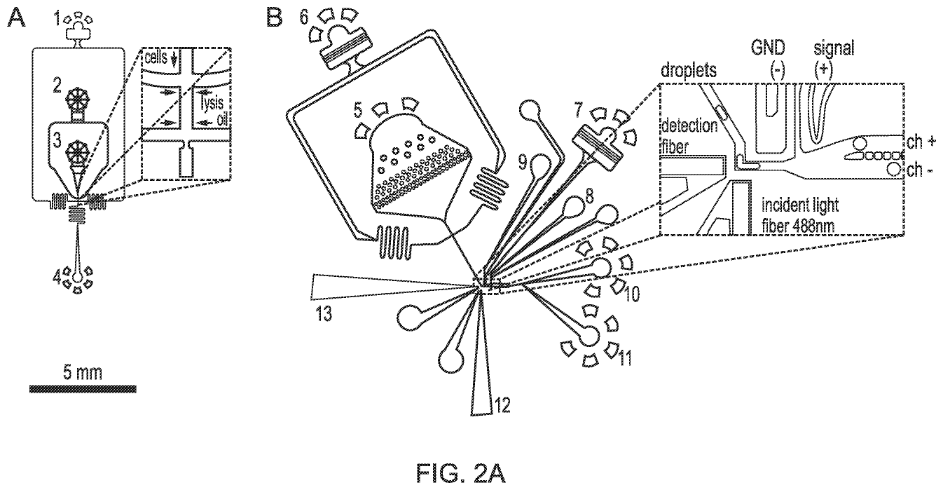

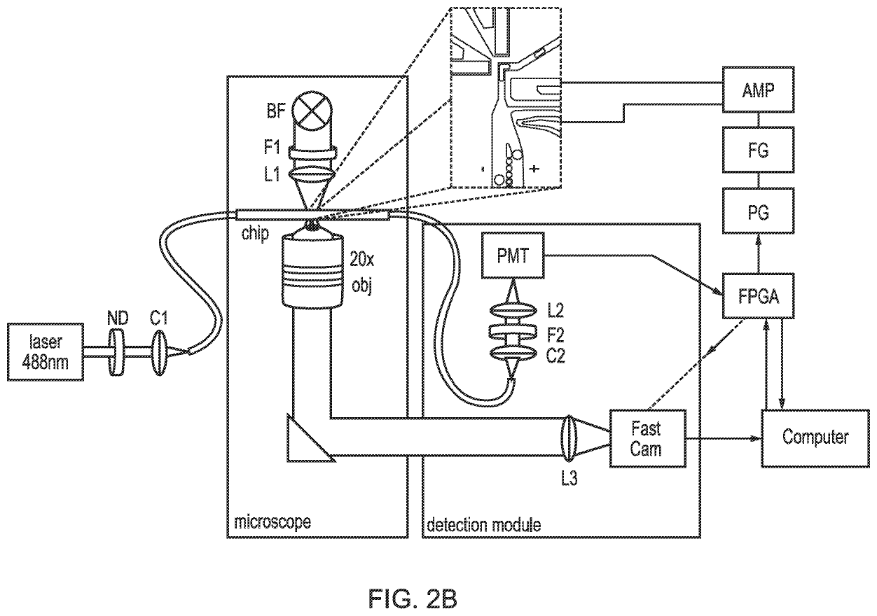

As illustrated on FIG. 1, the pooled metagenomic DNA was transformed into E. coli and cells encapsulated into ˜2 pL droplets under Poisson distribution of λ=0.35 (˜70.4% of the droplets were empty, ˜24.6% of the droplets contained 1 cell and ˜4.3% of the droplets contained 2 cells) together with lysis buffer and (1) fluorescein dihexanoate substrate. The generated droplets were incubated off-chip at room temperature and after incubation the emulsions were re-injected into the sorting chip and sorted over up to three days with a fluorescence-activated droplet sorting (FIG. 2B). From the sorted droplets, the plasmids were recovered, transformed into E. coli BL21 and the cells dispensed on LB agar plates containing tributyrin for secondary screening. The colonies which displayed a clear halo were selected for plasmid isolation and DNA sequencing, followed by gene annotation and biochemical characterisation in other to identify candidates displaying unique features.

Further experimental details are as follows:

Metagenomic library preparation for droplet encapsulation. 20 ng of DNA from the metagenomic library called “SCV” were transformed into E. coli 10G Elite (catalogue number 60051-2, Lucigen) yielded ca. 5×10⁷ variants on Luria Bertani (LB) agar plates containing 50μg/mL kanamycin (50× the library size). The plates were incubated at 37° C. overnight, then moved to room temperature (ca. 22° C.) for 48 h. The colonies were scraped from the plates using 3×3 mL liquid LB containing 50 g/mL kanamycin, cooled on ice, and washed three times by centrifugation at 5,000 g for 5 min and resuspension in buffer (50 mM TrisHCl pH 8.0, 100 mM NaCl, 50 μg/mL kanamycin, and one tablet of complete EDTA-free protease inhibitor (Roche) for a final volume of 50 mL). The washed cell suspension was diluted to OD₆₀₀ₙₘ 0.8 in buffer with 25% v/v Percoll (Sigma-Aldrich).

Droplet generation and incubation. Droplets were generated in a double flow-focusing junction (FIG. 1). The first aqueous stream contained the cell suspension at OD₆₀₀ₙₘ 0.8; and the second aqueous stream was composed of 20 μM of fluorescein-dihexanoate, 0.4×BugBuster (BugBuster 10×Protein Extraction Reagent, catalogue number 70921-4, Merck-Millipore), and 27 to 33 U/μL rLysozyme (catalogue number 71110, Merck-Millipore) in buffer containing 100 mM NaCl and 100 mM Tris-HCl pH8.0. For the oil phase, it was used HFE-7500 (3M) containing 1% w/w fluorosurfactant-008 (RAN Biotechnologies). The flow-rates were 50 μL/h for each aqueous and 500 μL/h for the oil phase resulting in droplet volumes of about 2 pL and an expected droplet occupancy of λ=0.35. The droplets were collected into an inverted 500 μL microcentrifuge tube which was modified by inserting tubing at the top and bottom through access holes which were then sealed with adhesive glue (Scotch-Weld PR1500, 3M). The droplets were incubated under quiescent conditions at room temperature (ca. 22° C.) and in the dark for up to 72 hours.

Fluorescence-activated droplet sorting (FADS). Droplets were injected from the modified microcentrifuge tube into the sorting chip (FIG. 1) at 5 μL/h and spaced with fluorous oil HFE7500 at 100 μL/h resulting in a sorting frequency of about 300 Hz. The droplet injection rate was increased up to 15 μL/h (to a rate of 2,000 Hz) and the oil flow adjusted to create enough separation to sort single droplets. The chip was monitored using the microscope (IX73, Olympus) light source with a longpass filter (593 nm, BrightLine Semrock) and a high speed camera (Miro eX4, Phantom Research). To measure droplet fluorescence, a laser beam (488 nm, 30 mW, 85 BCD 30 Melles-Griot, attenuated with ND 1.0) was expanded 10× and focused onto the microfluidic channel upstream of the sorting junction via a dichroic mirror (495 nm, Olympus). The induced fluorescence was collected by an air objective (LUCPlanFLN 40x/0.6, Olympus), passed through a longpass filter (488 nm, RazorEdge Semrock), a dichroic mirror (555nm, Thorlabs), and finally a bandpass filter (525/28nm, BrightLine Semrock) before reaching the detector the photomultiplier tube (PM002, Thorlabs). The signal was fed to an anaolog-in pin of a Virtex-5 LX30 FPGA (PCIe-7841R, NI). The FPGA quantified the width at signal threshold, width at half-maximum, area, and amplitude of each droplet signal. The droplet data was streamed to a custom LabView program for visualisation and the setting of sorting thresholds. If a droplet met the sorting criteria, trigger was sent to the pulse generator, which created a 5 V pulse with 500 μs width (TGP110, Thurlby Thandar Instruments). This pulse controlled a function generator (20 MHz DDS Function Generator TG 2000, TTi) working in external gated mode, generating a 10 kHz square signal at an adjustable amplitude (7 to 9 V), which was then amplified 100 times with a voltage amplifier (TREK 601c) to actuate the sorter. The electrodes were made by filling channels with salt solution (5 M NaCl) and connected to the amplifier via syringes (19).

Plasmid recovery and high-efficiency transformation. To the sorted droplets collected into a low binding DNA tube (catalogue number 183225J, Eppendorf), 100 μL of 2 ng/μL salmon sperm DNA (catalogue number 15632-011, Invitrogen) and 100 μL of 1H,1H,2H,2H-Perfluorooctanol (97%, catalogue number B20156.30, Alfa Aesar) were added. The tube was vortexed for 30 seconds and centrifuged for 1 min at 1,000 g. The aqueous layer was transferred to a fresh low binding DNA tube using low retention pipette tips (maxymum recovery, Axygen) and the same procedure repeated another two times with 100 μL of the salmon sperm solution and 100 μL of 1H,1H,2H,2H-Perfluorooctanol. The resulting 300 μL of recovered DNA solution was purified using Zymo Clean and Concentrate-5 kit (catalogue number D4004, Zymo Research) according to the manufacturer's protocol. The DNA was eluted with 12 μL of ultra-pure distilled water (catalogue number 10977-035, Invitrogen) after an incubation time of 5 minutes.

Secondary screen with tributyrin. Of the recovered solution, 2 μL of recovered plasmids were electrotransformed into E. coli 10G Elite (catalogue number 60051-2, Lucigen) and cells were recovered with 1 mL of recovery medium (catalogue number 80026-1, Lucigen) for 1 h at 37° C. and 450 rpm. The transformed cells were plated on LB agar plates containing 50 μg/mL kanamycin and 1% tributyrin (97%, catalogue number W222305, Sigma Aldrich) and the plates were incubated for one day at 37° C. followed by room temperature. The plates were daily checked for the presence of clear halos around the colonies and the positive ones selected for miniprep with Genejet plasmid miniprep kit (catalogue number K0503, ThermoFisher) for DNA sequencing.

### RESULTS: New Esterases From Different Regions of Sequence Space Have Been Discovered

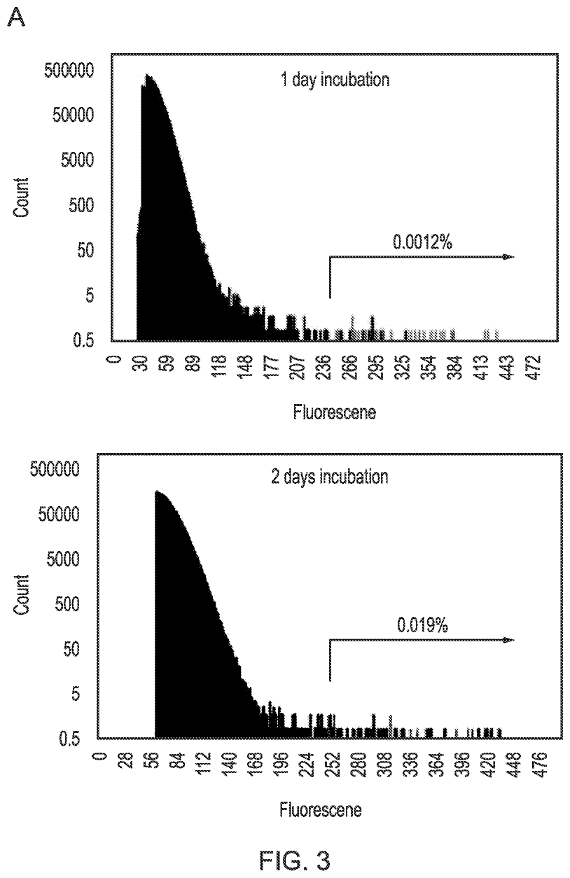

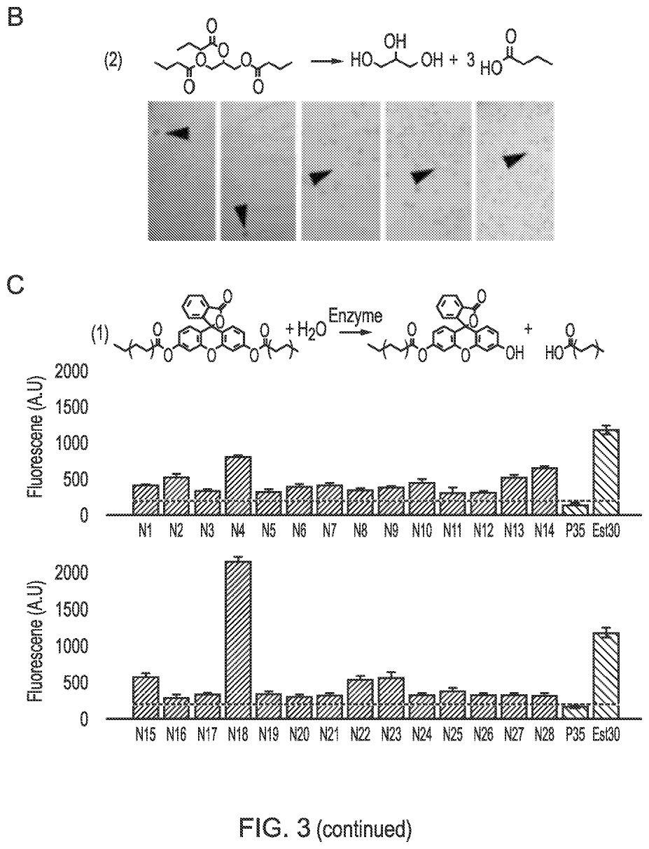

In total, two screening campaigns were performed and ˜ 30.6 million droplets were screened, 15.7 million droplets were screened over campaign I and 14.9 droplets were screened in campaign Il across two and three days, respectively. Considering the applied Poisson distribution of λ=0.35, 28.9% of the droplets contained 1 or 2 cells, therefore ˜8.8 million droplets contained a metagenomic plasmid. Within these two screening campaigns the metagenomic library was covered seven times, allowing the identification of 34 colonies displaying clear halos in campaign I. Campaign I is shown in FIG. 3.

The DNA sequencing analysis of the candidates from campaign I showed that 27 candidates were identified as potential positive hits, 4 candidates couldn't be sequenced due to low quality reads, 2 candidates were vectors without inserts and 1 candidate showed to be a false positive. Regarding campaign II, although 18 candidates were identified as potential positive hits, just one was confirmed as a new hit (RR11), as the other candidates were previously identified over campaign I. The activity of the potential positive candidates was tested towards fluorescein dihexanoate and the reaction monitored over time with a plate reader. As shown, the candidates displayed higher activity compared to the negative control P35, the activity of P35 is illustrated as grey bar and the dotted grey line represents the comparison of this negative control with the metagenomic hits identified.

In order to obtain the full-length contig of the eDNA insert from each metagenomic hit, the primer walking strategy was applied and primers were designed in a way that after up to four rounds of DNA sequencing the full-length sequence of each metagenomic insert was obtained. The analysis of the full-length contigs showed that from the 28 potential hits (27 candidates from campaign I and 1 candidate from campaign II), 10 hits were unique over campaign I and 1 was unique over campaign II, giving a sum of 11 unique metagenomic-derived hits.

The contig analysis of candidate N20 allowed the identification of ORF n2001, which was addressed to the family of protein of unknown function, DUF3089 (Pfam code PF11288), a small family with 630 members on Pfam database.

From the 11 unique metagenomic hits identified, 13 ORFs were assigned to esterase/lipase function. The sequence comparison of these 13 ORFs against the NCBI (metagenomic proteins, env_nr) and the UniprotKB databases showed that 69% and 30.7% of the ORFs share a sequence similarity lower or higher than 50%, respectively. Amongst the ORFs with lowest sequence identity is rr1102 that showed 38% of sequence similarity with a hypothetical protein from hydrocarbon metagenome (NCBI accession code KUG22667.1) and 64% with uncharacterized protein from Oribacterium sp. (UniprotKB accession code A0A1H3Y0C3). The comparison of 13 ORFs towards the Pfam and ESTHER databases shows that 8 and 9 families could be assigned according to Pfam and ESTHER, respectively. This shows that diverse families could be explored during the screening process. In addition, according to the current proposed classification of lipolytic enzymes the present screening platform allowed access to 6 different families (II, III, V, VII, X, XIX). 4 candidates couldn't be precisely address to any family.

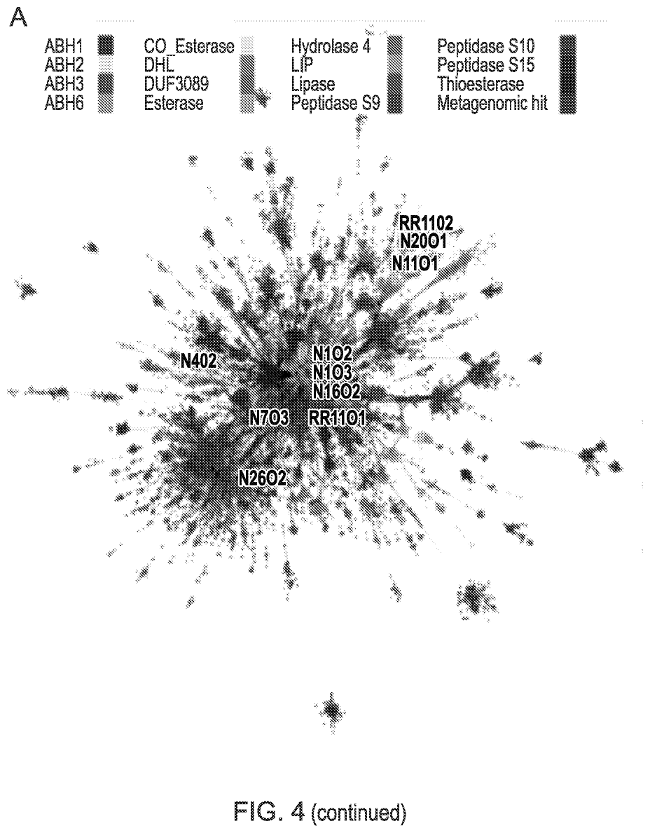

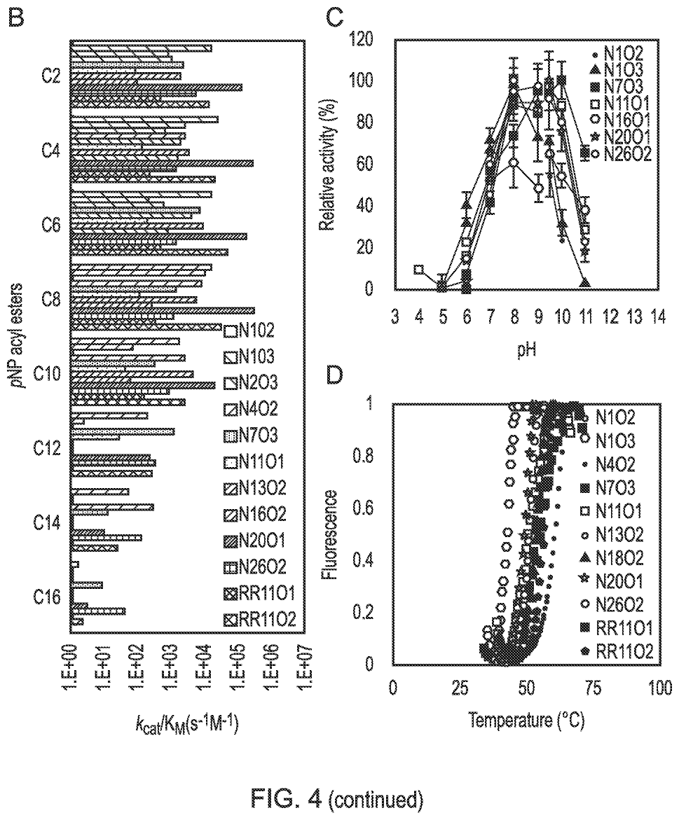

To address the activity of the metagenomic hits, each ORF was individually cloned into an expression vector and the corresponding recombinant proteins tested against p-NP acyl esters with different chain lengths. As expected, the highest catalytic efficiency was observed towards esters with medium chain length (e.g. butyrate, hexanoate and octanotate), in agreement to the chain length applied during the droplet screening with (1). Similar observations were made for the pH, pH 8.0 was the optimum pH observed for all candidates. Additionally, the melting temperature was also investigated showing a mesophilic profile for all metagenomic hits tested. These results are shown in FIG. 4.

The catalytic promiscuity of metagenomic hits were also investigated using a range of substrates with para-nitrophenol as the leaving group as set out below:

TABLE 4

| Substrate | p-NP esters | Abbreviation | Molecule |
|---|---|---|---|
| 3a | p-NP acetate | C2 | 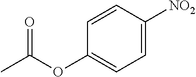 |
| 3b | p-NP butyrate | C4 | 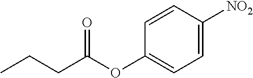 |
| 3c | p-NP hexanoate | C6 | 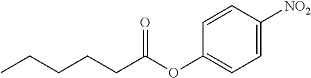 [SMILES: CCCCCC(=O)Oc1ccc([N+](=O)[O-])cc1] |
| 3d | p-NP octanoate | C8 | 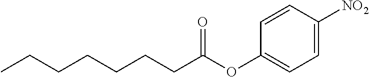 [SMILES: CCCCCCCC(=O)Oc1ccc([N+](=O)[O-])cc1] |
| 3e | p-NP decanoate | C10 | 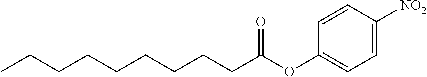 [SMILES: CCCCCCCCCC(=O)Oc1ccc([N+](=O)[O-])cc1] |
| 3f | p-NP dodecanoate | C12 | 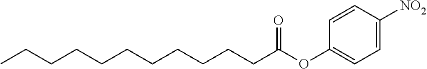 [SMILES: CCCCCCCCCCCC(=O)Oc1ccc([N+](=O)[O-])cc1] |
| 3g | p-NP myristate | C14 | 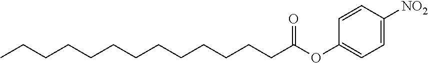 [SMILES: CCCCCCCCCCCCCC(=O)Oc1ccc([N+](=O)[O-])cc1] |
| 3h | p-NP palmitate | C16 | 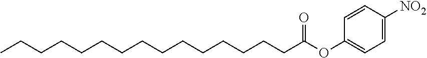 [SMILES: CCCCCCCCCCCCCCCC(=O)Oc1ccc([N+](=O)[O-])cc1] |

As expected all candidates showed activity towards p-NP esters with short chain length and also towards long chain length. Furthermore, almost all candidates showed thioesterase activity.

Activity against the compounds in Table 4 is described for selected enzymes:

TABLE 5a

| | N20O1 | | | | |
|---|---|---|---|---|---|
| | | | k_{cat}/K_{M} | | |
| Substrate | K_{M} (M) | k_{cat} (s⁻¹) | (s⁻¹M⁻¹) | [E] | |
| 3a | 3.8 × 10⁻³ ± | 5.2 × 10² | 1.3 × 10⁵ | 6.2 | nM |
| | 5.7 × 10⁻¹ | | | | |
| 3b | 2.2 × 10⁻³ ± | 6.3 × 10² | 2.8 × 10⁵ | 1 | nM |
| | 1 | | | | |
| 3c | 1.4 × 10⁻³ ± | 2.5 × 10² | 1.8 × 10⁵ | 6.2 | nM |
| | 1.63 | | | | |
| 3d | 1.1 × 10⁻³ ± | 3.7 × 10² | 3.1 × 10⁵ | 12.5 | nM |
| | 2.5 × 10⁻¹ | | | | |
| 3e | 1.5 × 10⁻³ ± | 3.1 × 10¹ | 2.0 × 10⁴ | 25 | nM |
| | 2 × 10⁻¹ | | | | |
| 3f | 2 × 10⁻³ ± | 5.0 × 10⁻¹ | 2.4 × 10² | 5 | uM |
| | 1.9 | | | | |
| 3g | 4.4 × 10⁻³ ± | 4.2 × 10⁻² | 9.7 | 25 | uM |
| | 5.0 × 10⁻¹ | | | | |
| 3h | 4.8 × 10⁻³ ± | 1.6 × 10⁻² | 3.3 | 25 | uM |

3.6

TABLE 5b

| | RR11O2 | | | | |
|---|---|---|---|---|---|
| | | | k_{cat}/K_{M} | | |
| Substrate | K_{M} (M) | k_{cat} (s⁻¹) | (s⁻¹M⁻¹) | [E] | |
| 3a | 3.0 × 10⁻² ± | 3.9 × 10² | 1.3 × 10⁴ | 31.2 | nM |
| | 4.4 | | | | |
| 3b | 2.2 × 10⁻³ ± | 4.6 × 10¹ | 2.0 × 10⁴ | 31 | nM |
| | 5.9 × 10⁻¹ | | | | |
| 3c | 7.2 × 10⁻⁴ ± | 3.5 × 10¹ | 4.9 × 10⁴ | 31.2 | nM |
| | 2.3 × 10⁻¹ | | | | |
| 3d | 3.7 × 10⁻⁴ ± | 1.1 × 10¹ | 3.0 × 10⁴ | 31.2 | nM |
| | 4.1 × 10⁻² | | | | |
| 3e | 2 × 10⁻³ ± | 5.4 | 2.7 × 10³ | 125 | nM |
| | 1.9 × 10⁻¹ | | | | |
| 3f | 2.8 × 10⁻³ ± | 7.8 × 10⁻¹ | 2.7 × 10² | 396 | nM |
| | 4.1 × 10⁻¹ | | | | |
| 3g | 1.5 × 10⁻³ ± | 3.9 × 10⁻² | 2.5 × 10¹ | 23.2 | uM |
| | 2 × 10⁻¹ | | | | |
| 3h | 7.4 × 10⁻³ ± | 1.7 × 10⁻² | 2.3 | 46.4 | uM |

2.1

### Example 2: Exploring Sequence Space Around New Enzyme Cluster Containing RR11O2 and N2001 Reveals Further Enzyme Cluster

The full-length fasta sequences were downloaded from Pfam (Pfam 32.0, https://pfam.xfam.org/, April 2019) for the largest families of each clan. To reduce the number of sequences (nodes) in the network, the individual families were clustered stepwise to 90%, 60% and 30% sequence identity using cd-hit and psi-cd-hit. The representative sequences for each cluster were combined with the esterase hit sequences into a single database, which was used for an all-versus-all alignment (Protein-Protein BLAST 2.6.0+). Each line in the output file defined an edge (alignment) connecting two nodes (representative sequence) of the network. Duplicate edges, self-loops and edges above a certain e-value were removed using a custom python script. The simplified network was then imported into CytoScape 3.7.1 for visualisation.

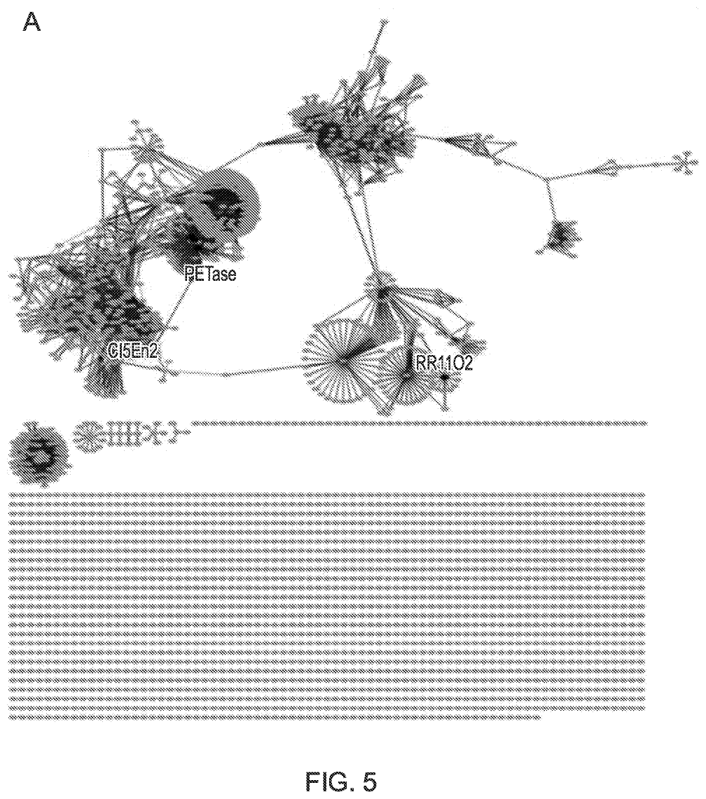

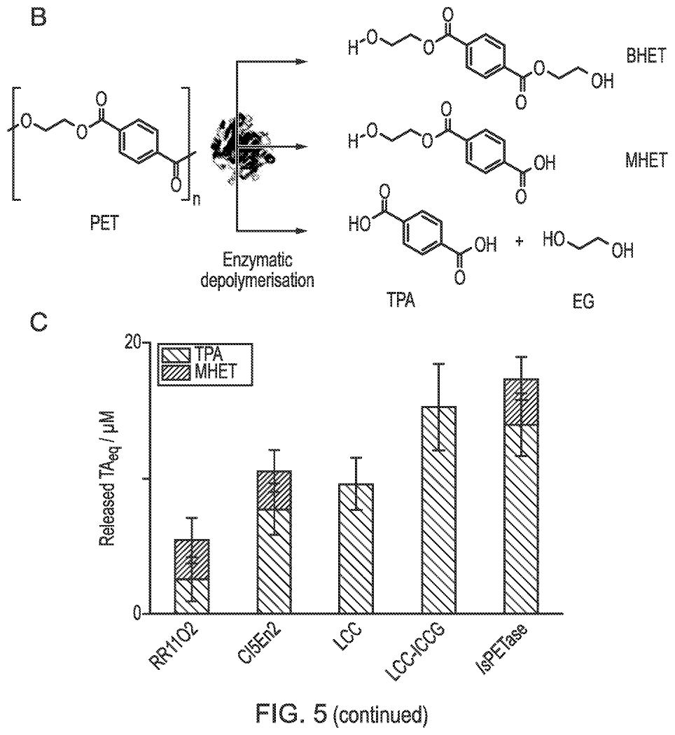

See FIG. 5. Enzyme CI5En2 was identified as part of a cluster between known PETases and the newly identified RR11O2.

The cl5en2 gene was amplified by PCR with Phusion High-Fidelity DNA Polymerase (ThermoFisher Scientific, F530L) using the forward 5′-GAAAACCTGTACTTCCAGGGTATGGCGGATTATGCGAAAG-3′ and reverse 5′-TGACACTATAGAATACTCAAGCTTACACCACAAAACGGCTAAATTC-3′ primers. The amplified fragment was cloned via slic cloning into pExp vector series previously digested with Bsal and Hindlll. The cloning was confirmed by DNA sequencing and the corresponding plasmids were used for transformation of E. coli BL21 (DE3). The transformed cells were grown at 37° C. for 16 h in 5 mL of LB media containing 100 μg mL-1 ampicillin and obtained cultures were used to inoculate 250 or 500 ml of LB (100 μg mL-1 ampicillin). Cells were grown at 37° C. until the OD600nm reached 0.5-0.6 and the protein expression was induced by addition of 400 μM IPTG at 20° C. for 20 h. After expression, cells were harvested by centrifugation (at 4000×g, for 20 minutes, at 10° C.) and resuspended in Tris-HCl (35 mL, 50 mM, pH 8.0). Cell lysis was performed in an Emulsiflex (Avestin). The extract was brought to 50 mM Tris-HCl PH 8.0, 250 mM NaCl, 10 mM imidazole, 10 mM β-mercaptoethanol, and 5% (v/v) glycerol and centrifuged at 8,000 xg for 45 min at 4° C. to remove cell debris. The supernatant was loaded onto 3 mL of Ni-NTA resin (Qiagen) which was pre-equilibrated with purification buffer [50 mM Tris-HCl PH 8.0, 250 mM NaCl, 10 mM imidazole and 5% (v/v) glycerol] and the recombinant proteins were eluted using a stepwise imidazole gradient. The soluble expression of the recombinant proteins was analysed by SDS-PAGE gel and the selected eluted fractions from column purification were combined and concentrated by centrifugation with Amicon Ultra-15 filters (Merck-Millipore), using the appropriate cut-off based on their molecular weight. In order to eliminate possible contaminants an additional protein purification step was performed by size-exclusion chromatography using the columns HiLoad 16/60 Superdex 75 or 200 (GE Healthcare) at 4° C. For the samples without apparent contaminants, buffer exchange was performed using a Sephadex G-25 PD10 column (Amersham Biosciences) following the manufacturer's protocol. All the recombinant proteins were stored in 50 mM Tris-HCl PH 8.0, 100 mM NaCl and 2.5% (v/v) glycerol and the concentrations of purified were determined by measuring absorbances at 280 nm using Nanodrop ND-1000 spectrophotometer (Nanodrop Technologies).

### Results

CL5En2 exhibits activity comparable to IsPETase and higher than LCC at 30° C. CI5En2 also showed activity against PET nanoparticles with different crystallinity.

### Example 3: Manufacture of Plastic Nanoparticles

False positives were identified with the screening method of Example 1 above in that some of the esterases identified did not have high activity against plastics despite their esterase activity. Therefore, a high throughput method was developed by the inventors which could more selectively pick out esterases with plastic degrading activity and could also be used to select optimised mutant enzymes via directed evolution.

The method uses plastic nanoparticles which were prepared as follows:

50 mg cryo-milled crystalline PET granules or amorphous PET film (product code ES303015, Goodfellow GmbH, London, UK) were dissolved in 1,1,3,3,3,-hexafluoro-2-propanol (5 mL) for at least one hour. This solution was added dropwise (1 mL/min) to double distilled H₂O (50 mL, cooled in an ice bath) until an accumulation of aggregated particles on the water surface was observed. At the same time the water was rigorously stirred using an Ultra Turrax® stirrer at 8000 rpm (IKA, Germany). Then the suspension was filtered using Whatman filter paper (8 μm diameter). Afterwards the remaining solvent was evaporated.

The following analytical methods were used to characterize the nanoparticles synthesized:
(i) The particle size distribution was evaluated by measuring the hydrodynamic diameter by dynamic light scattering on a Zetasizer NanoZS (Malvern, UK).
(ii) To directly measure nanoparticle size, grain size, size distribution, and morphology transmission electron microscopy (TEM) was used.
(ii) The crystallinity was determined using Differential Scanning calorimetry (DSC) (1.5.).
(iii) The particle concentration of the final suspension was determined by weighing of the pellet obtained from a 4 mL aliquot of plastic nanoparticle suspension generated by centrifugation and drying at 95° C. for at least 1 hour.

To generate higher concentrated nanoparticle solutions, the suspension was incubated at 37° C. in an open tube until the required density was reached.

To stabilize PET in the droplet, Tween-80 was used at a concentration of 0.1-0.5%. This provided stable particles even in the presence of buffer and enzyme or cell lysate.

Additional plastic nanoparticles have been formed from nylon. These were made as follows: 50 mg nylon 6 or nylon 66 were dissolved in 1,1,3,3,3,-hexafluoro-2-propanol (5 mL) for at least one hour. This solution was added dropwise (1 mL/min) to double distilled H2O (50 mL, cooled in an ice bath) supplemented with 0.05 or 0.1% SDS until an accumulation of aggregated particles on the water surface was observed. At the same time the water was rigorously stirred using an Ultra Turrax® stirrer at 8000 rpm (IKA, Germany). Then the suspension was filtered using Whatman filter paper (8 μm diameter). Afterwards the remaining solvent was evaporated.

### Results

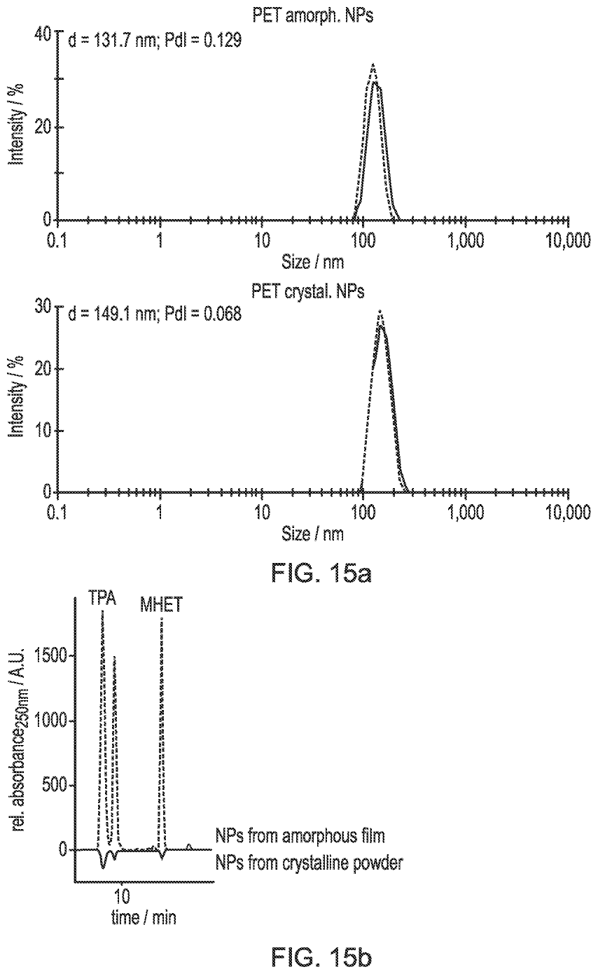

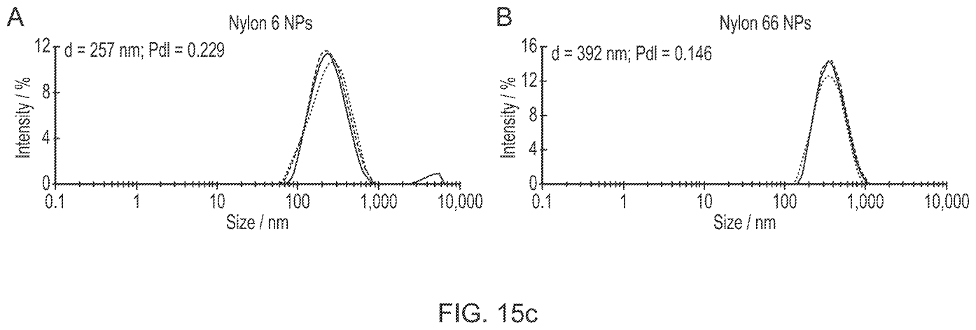

The results are shown in FIG. 15. When using the nanoparticles, no sedimentation was observed in the microfluidic droplets, even in droplet measurements taken after several weeks. Tween-80 was found to be a useful stabilizer (preventing aggregation between nanoparticles and the biomolecules in the droplet).

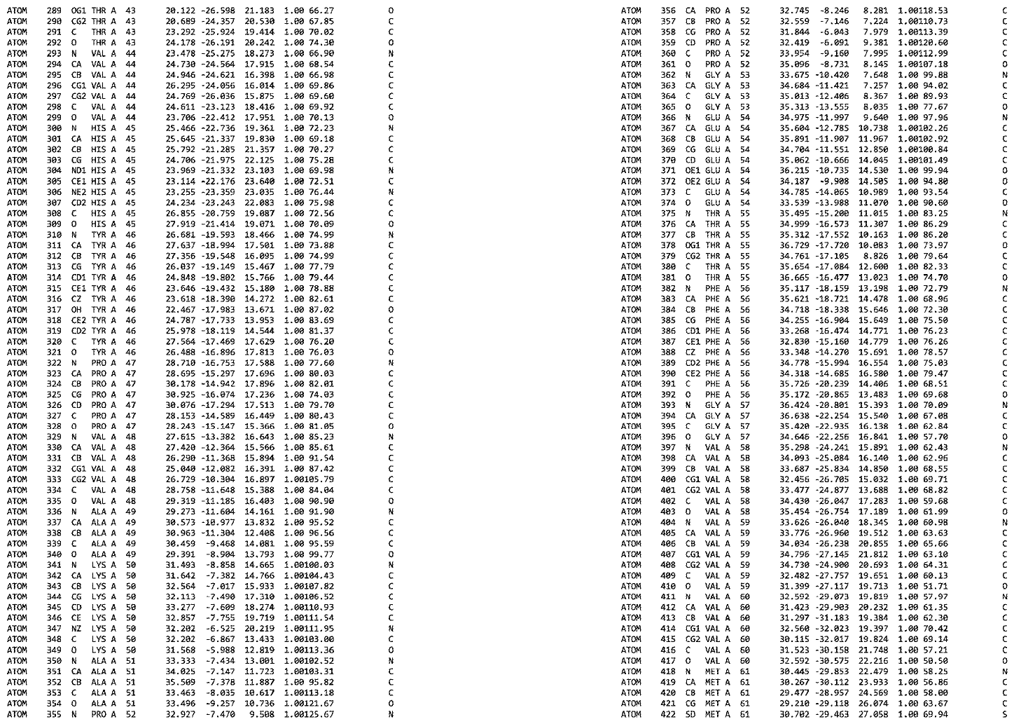

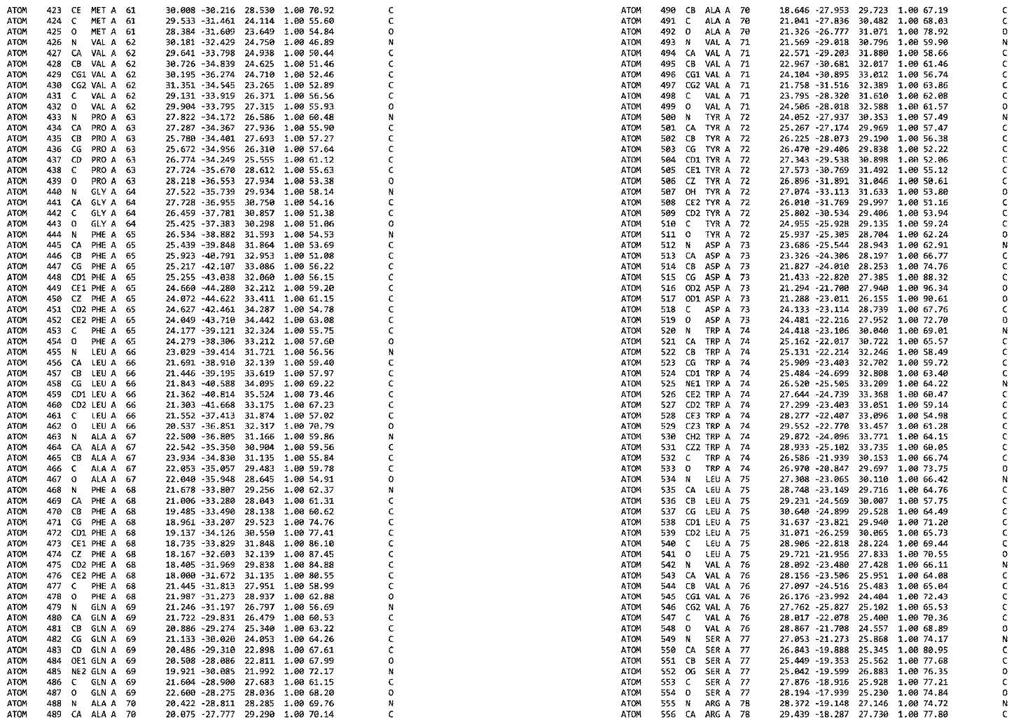

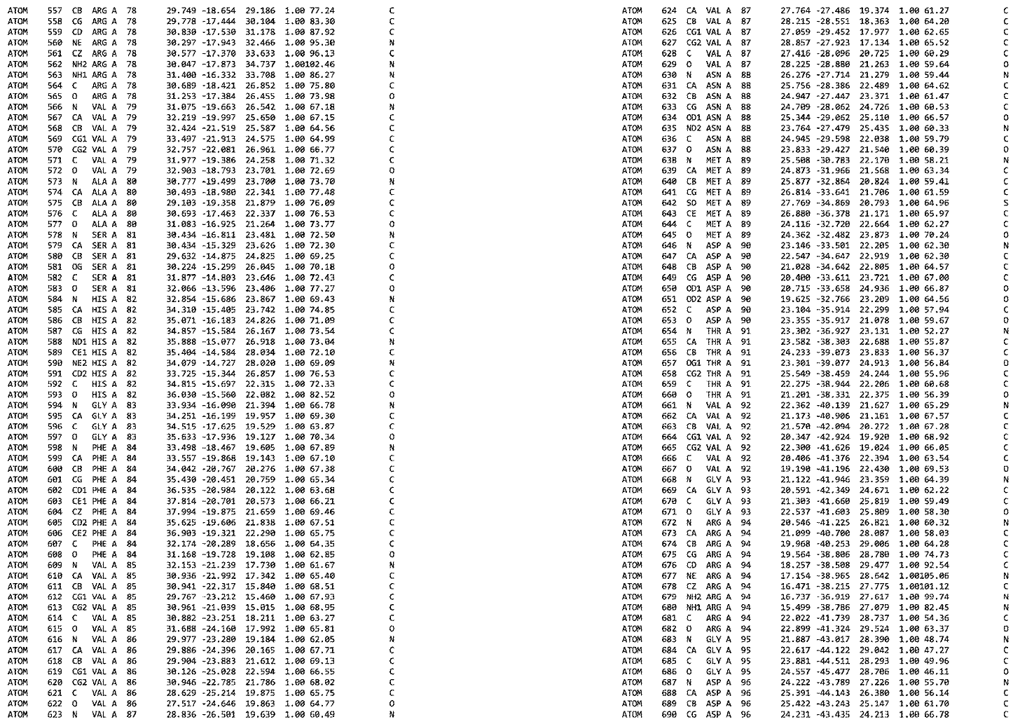

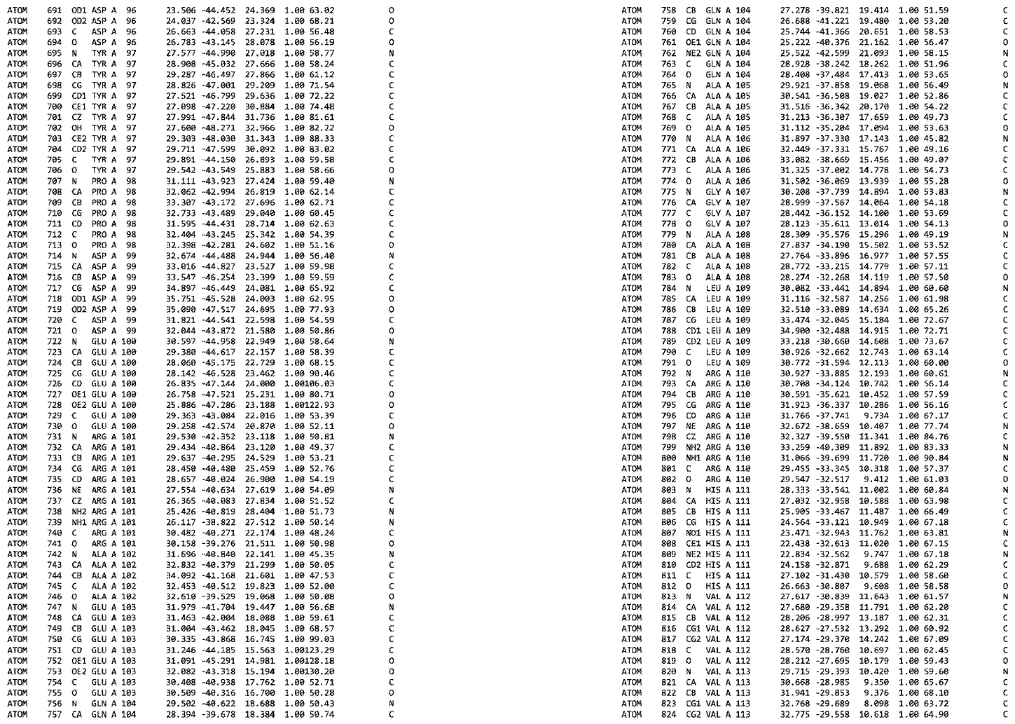

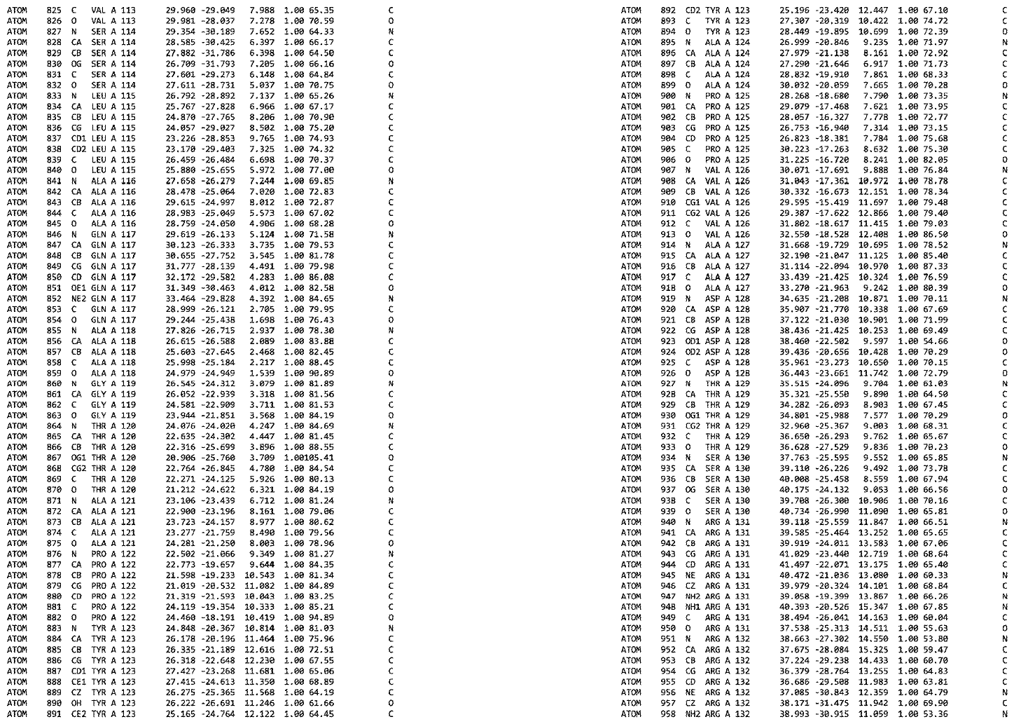

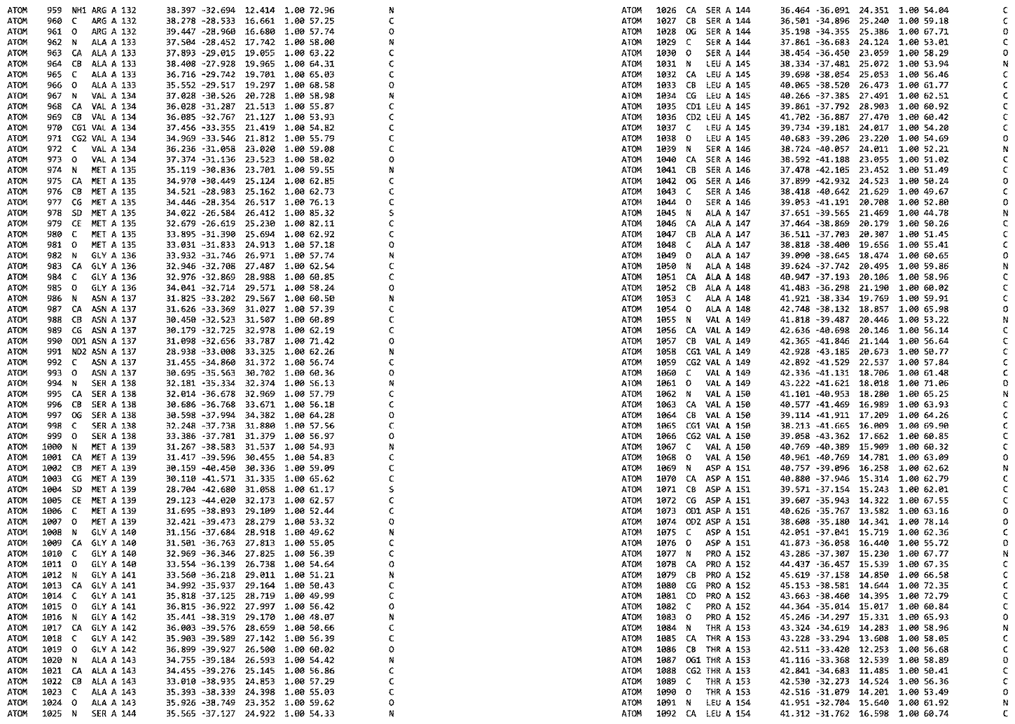

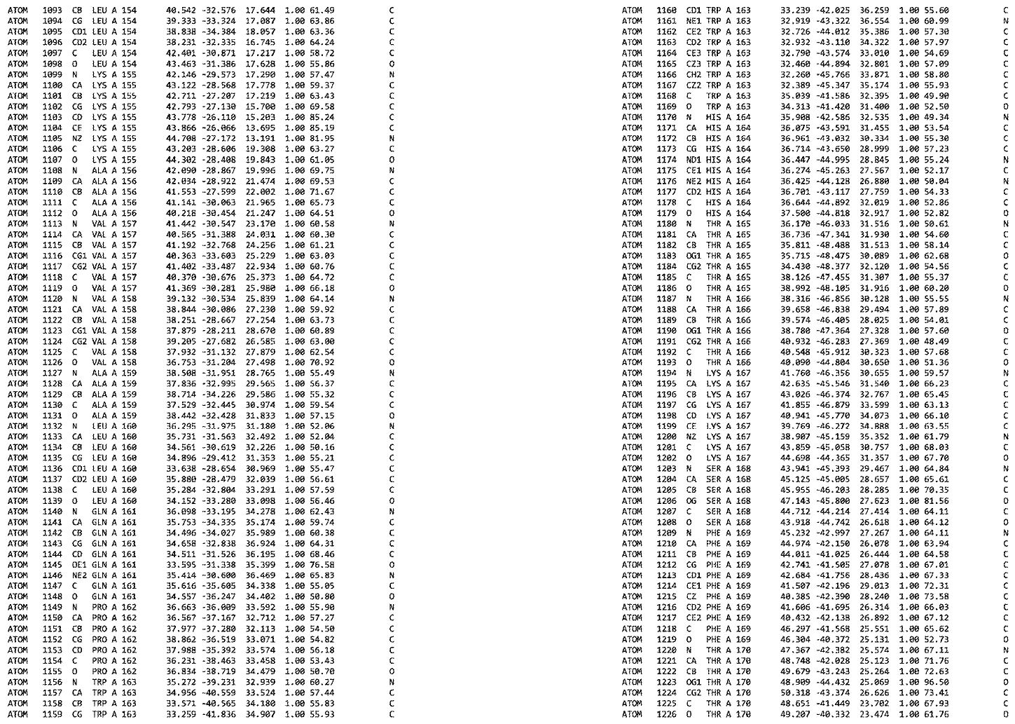

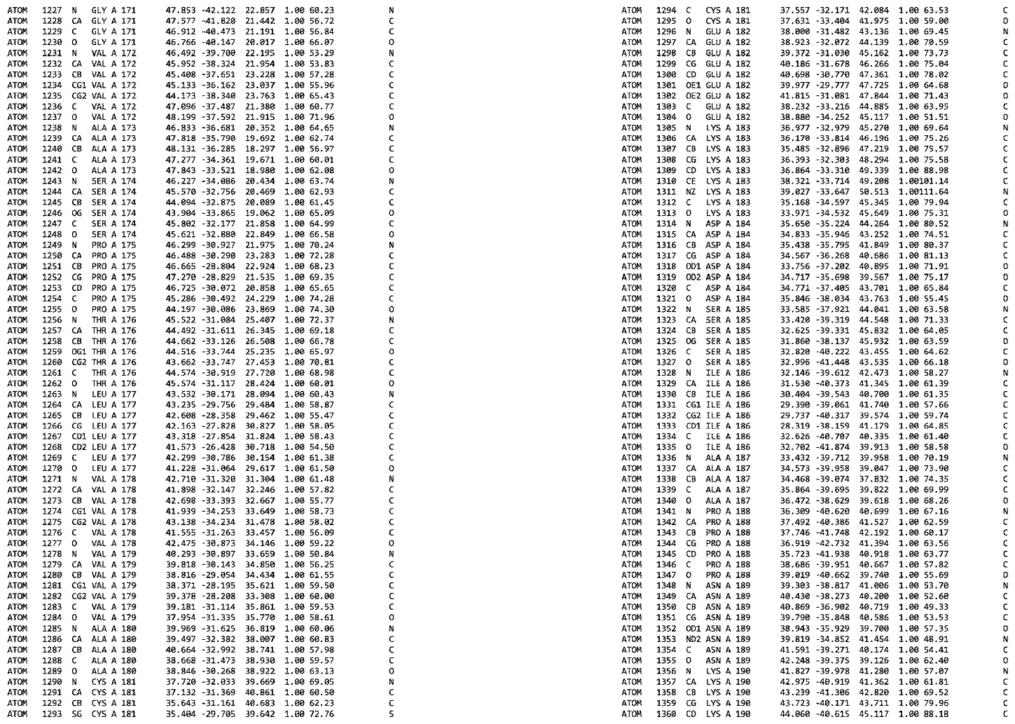

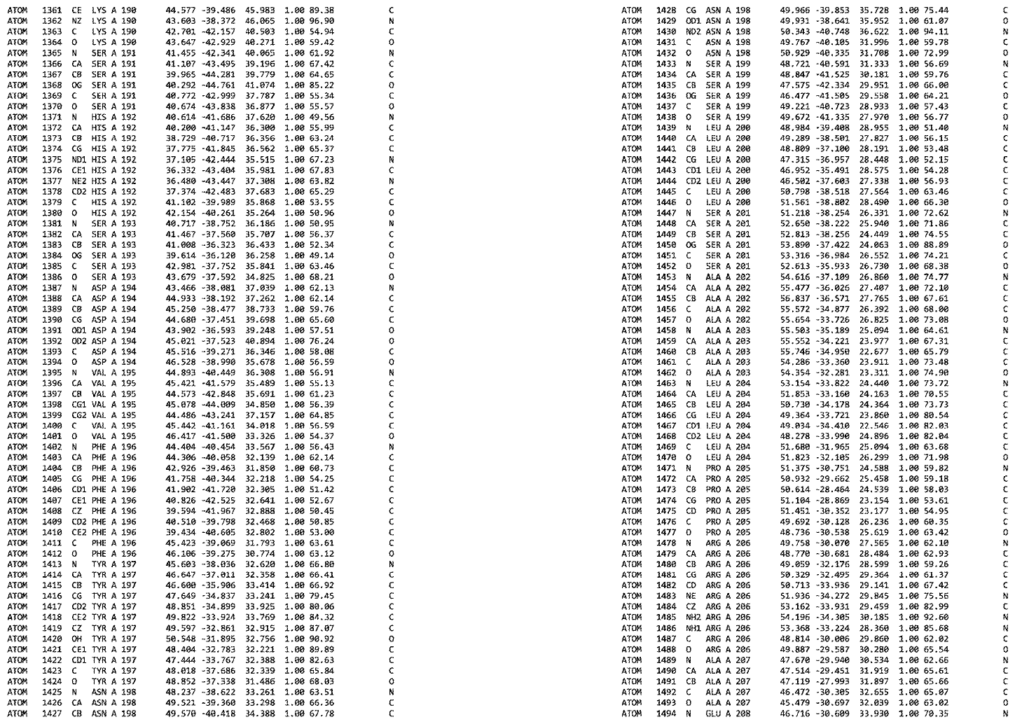

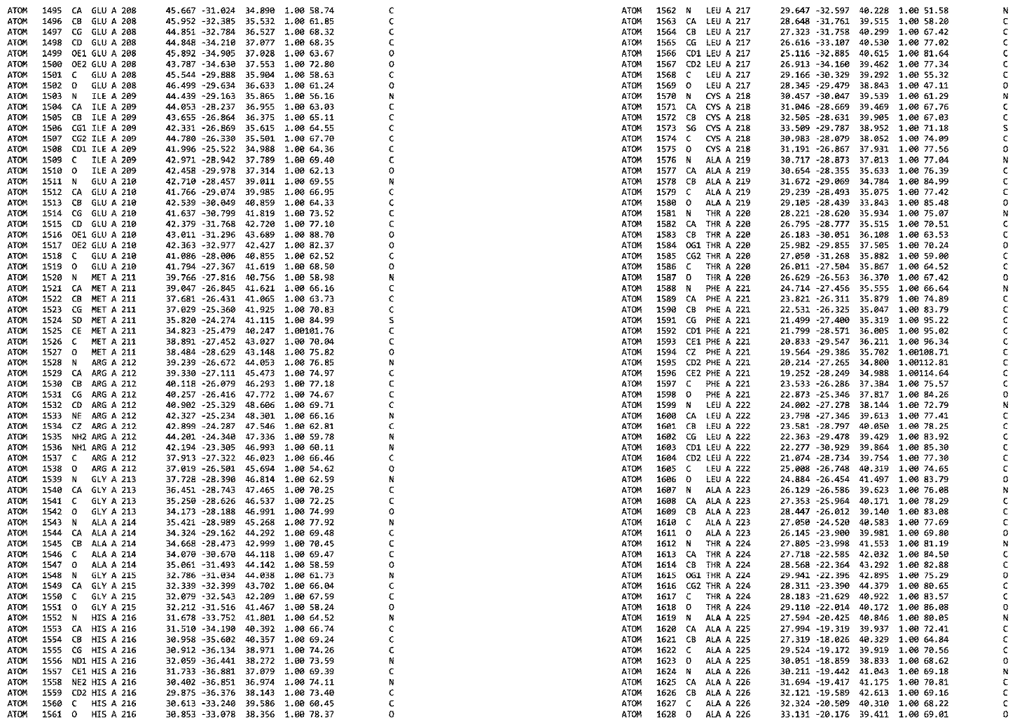

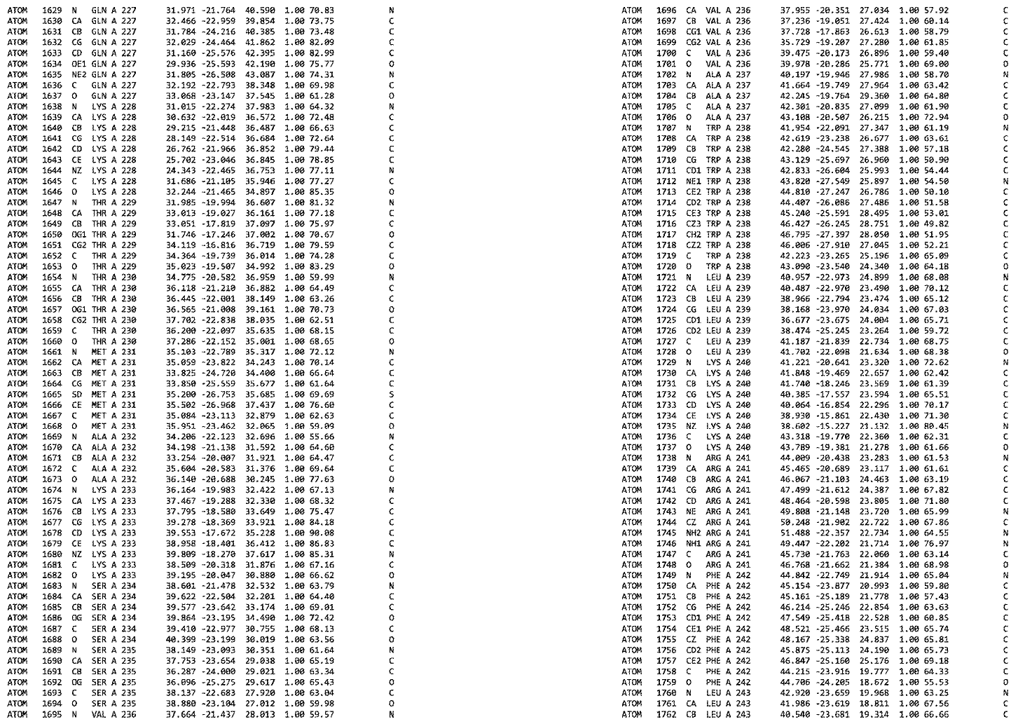

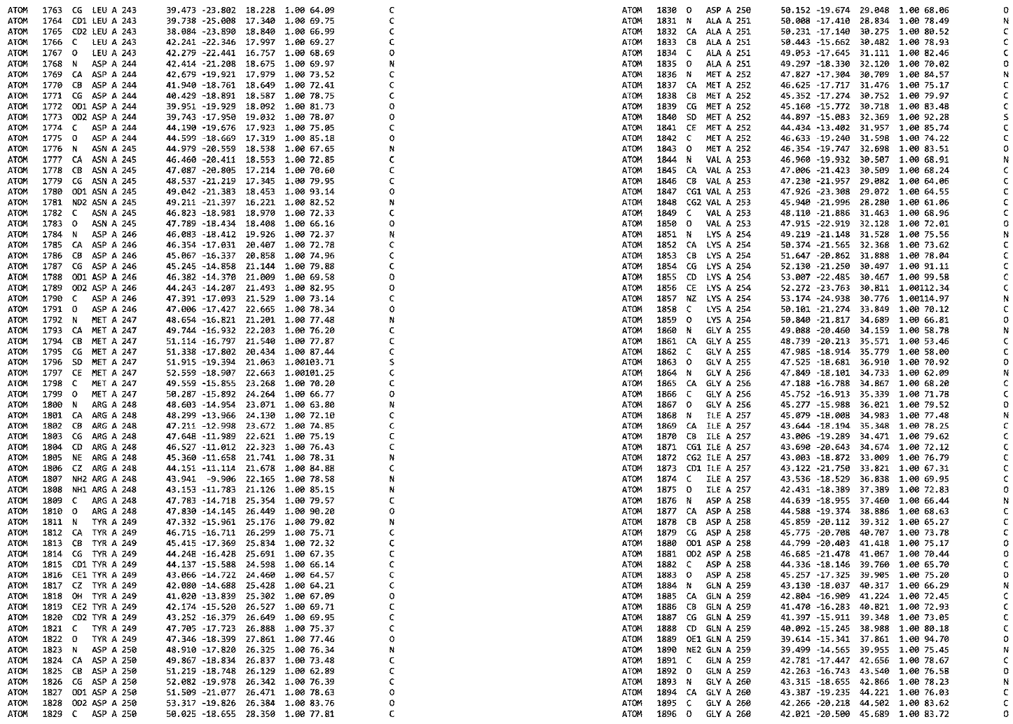

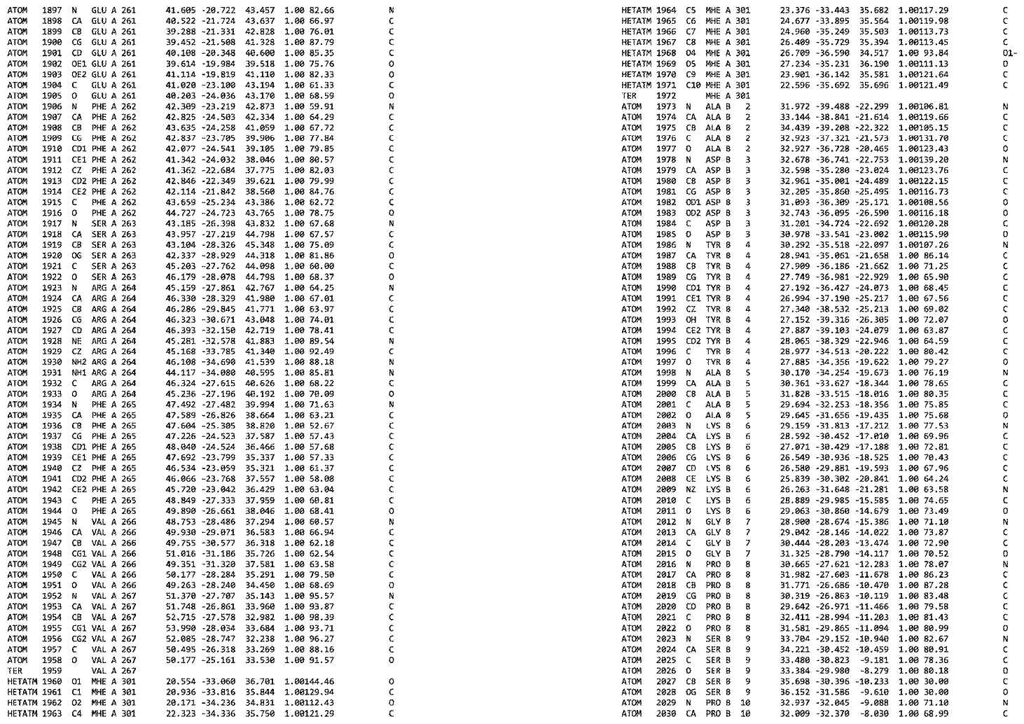

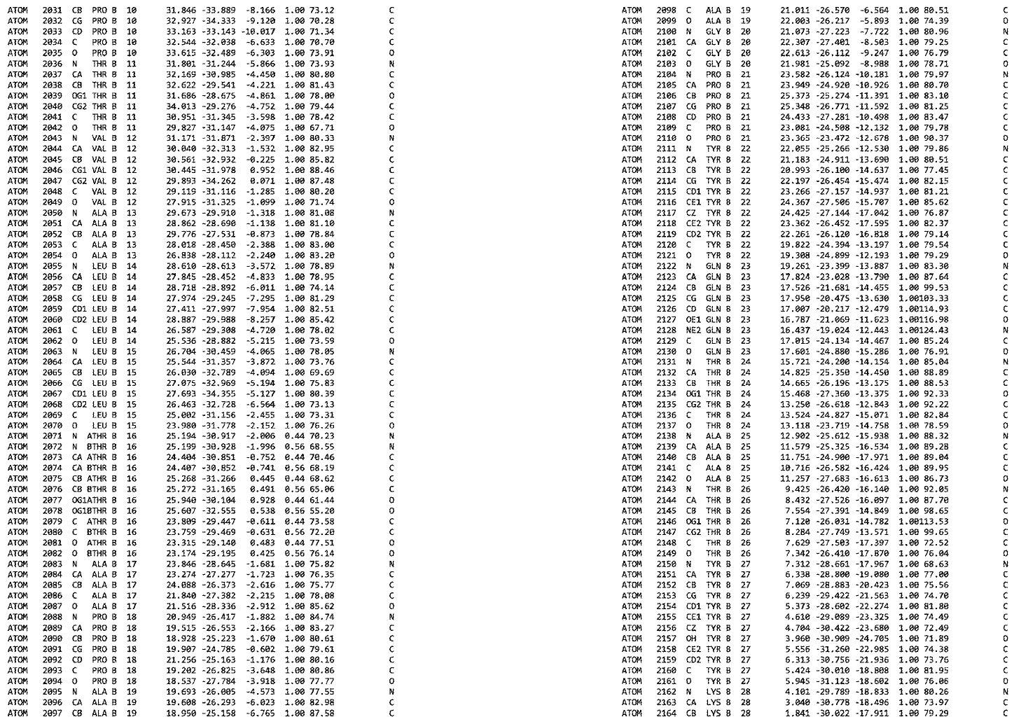

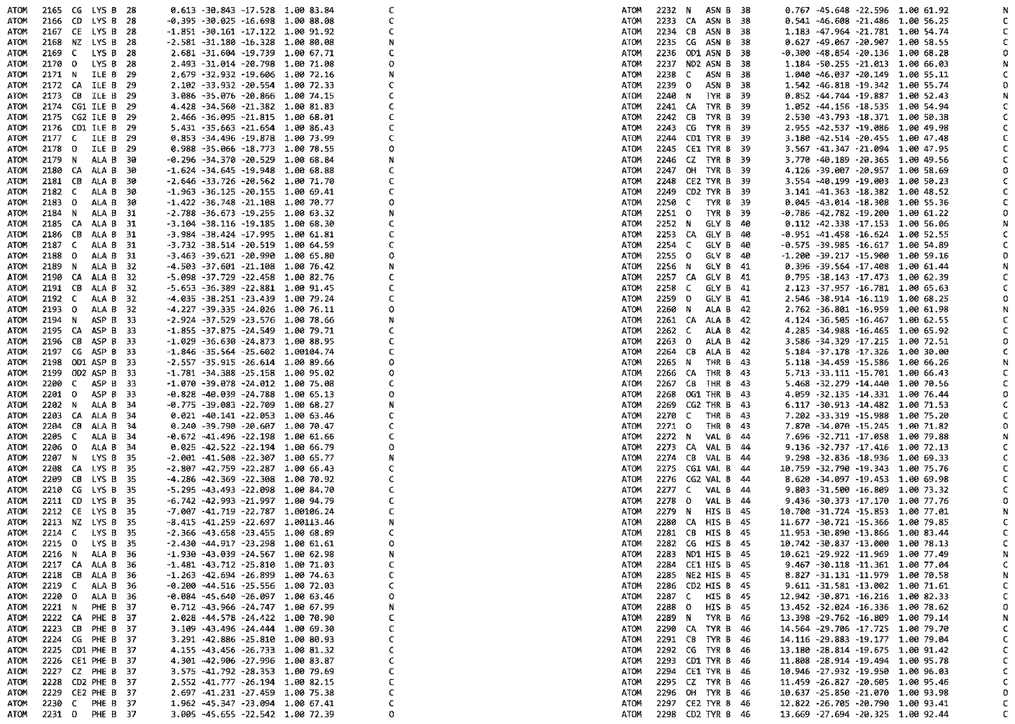

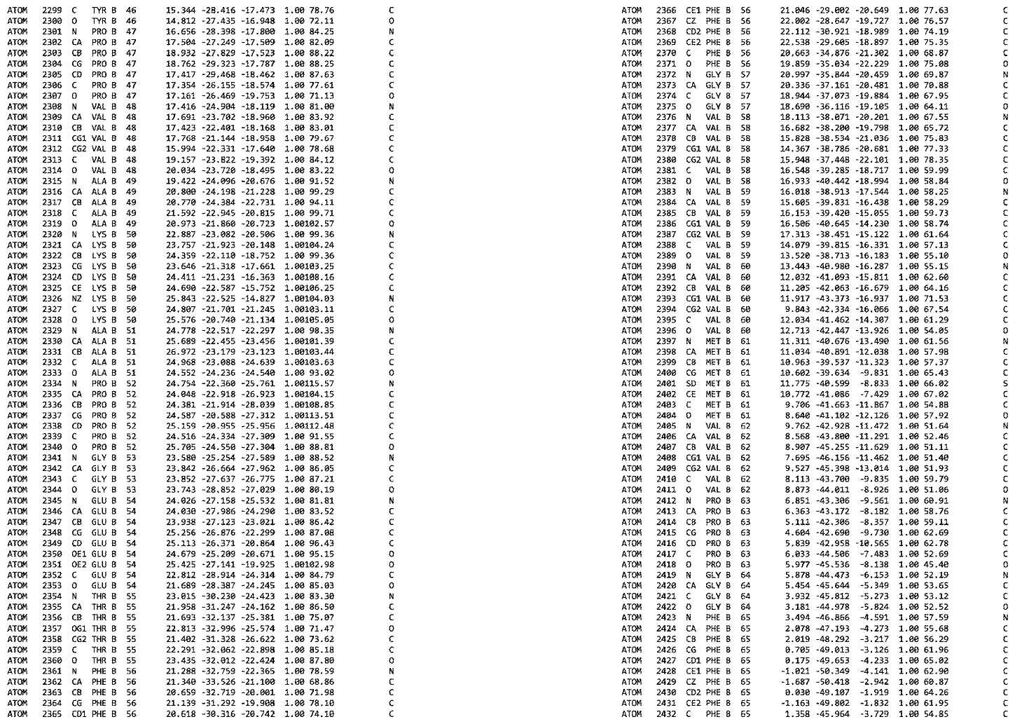

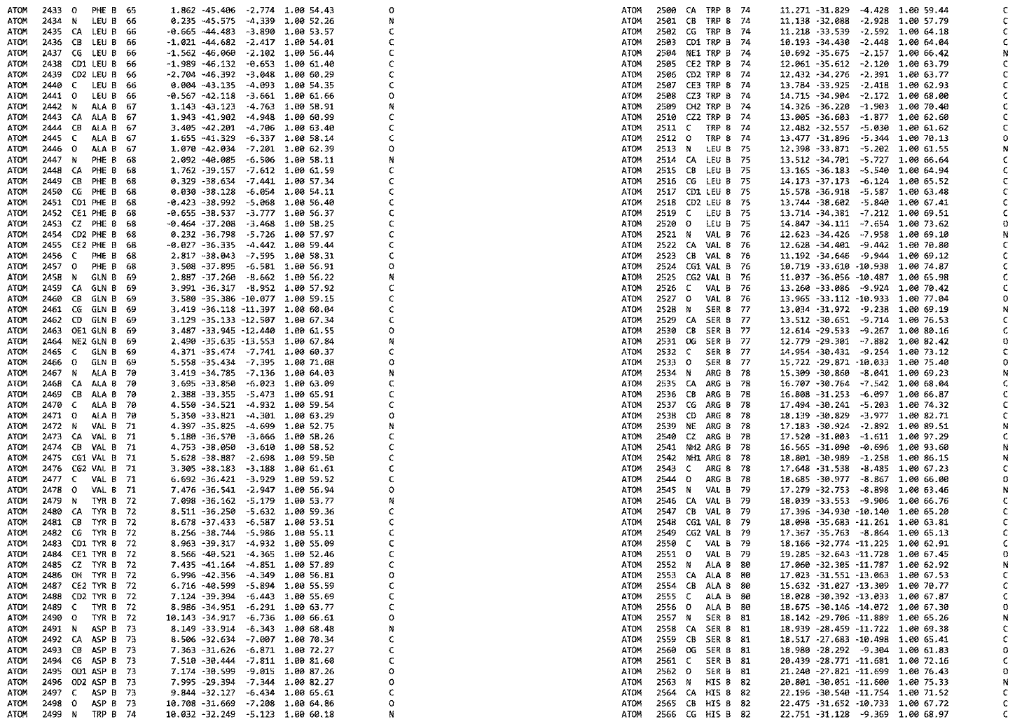

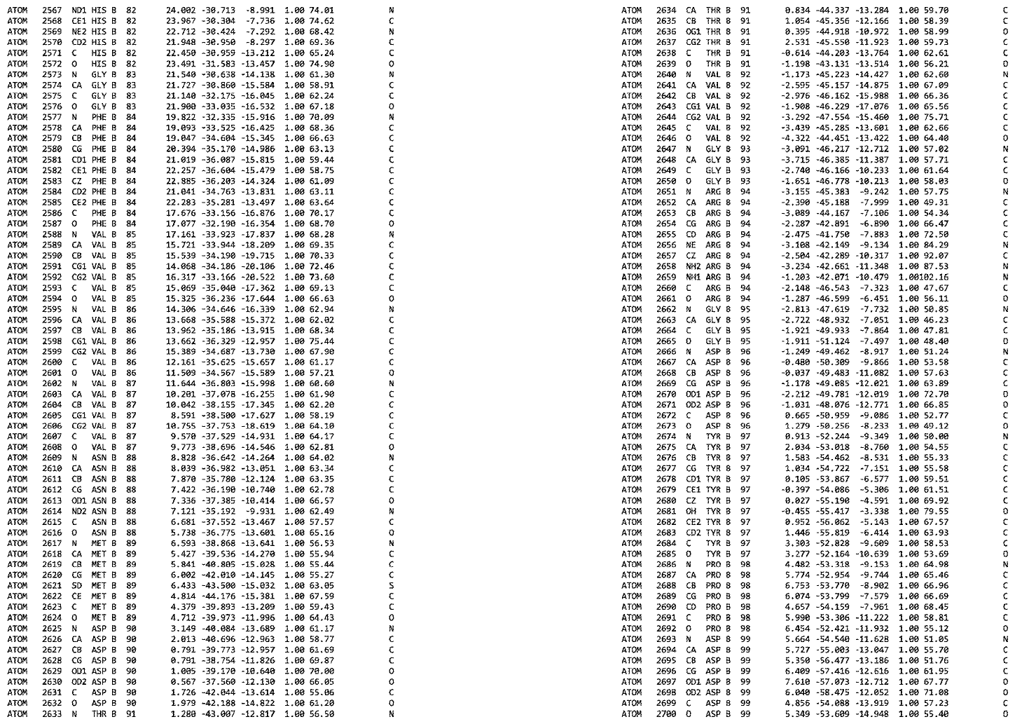

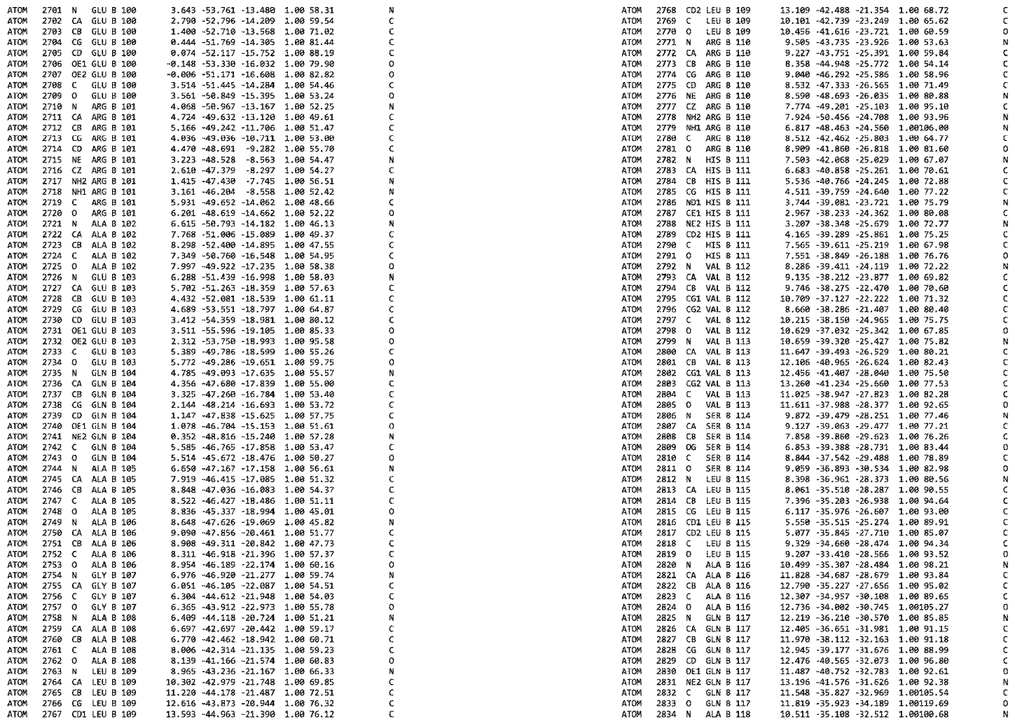

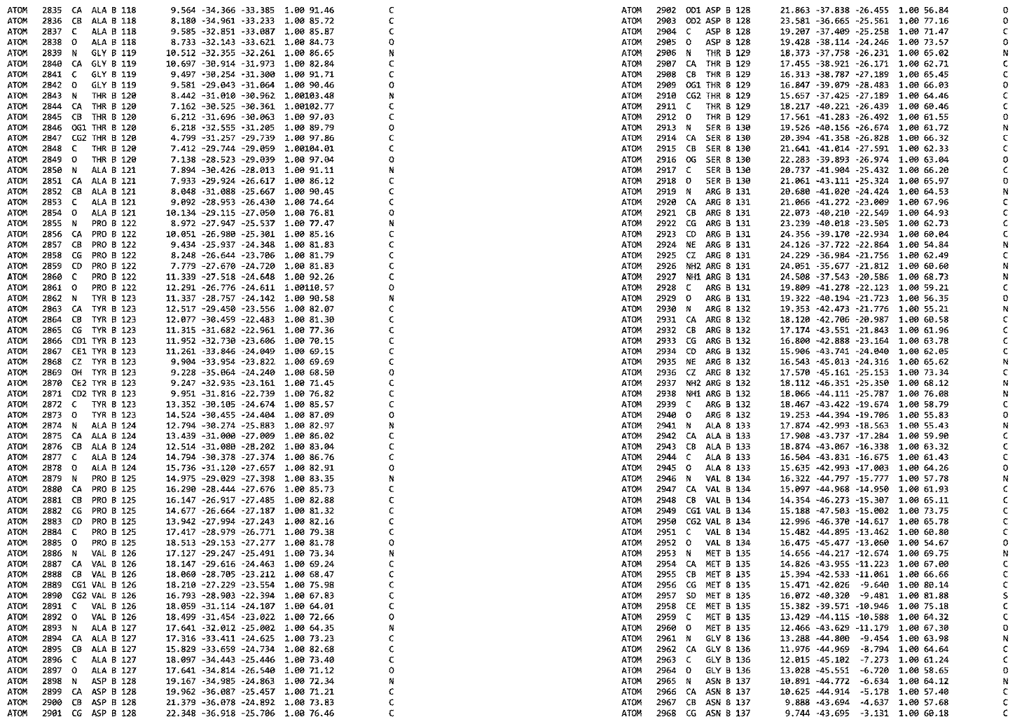

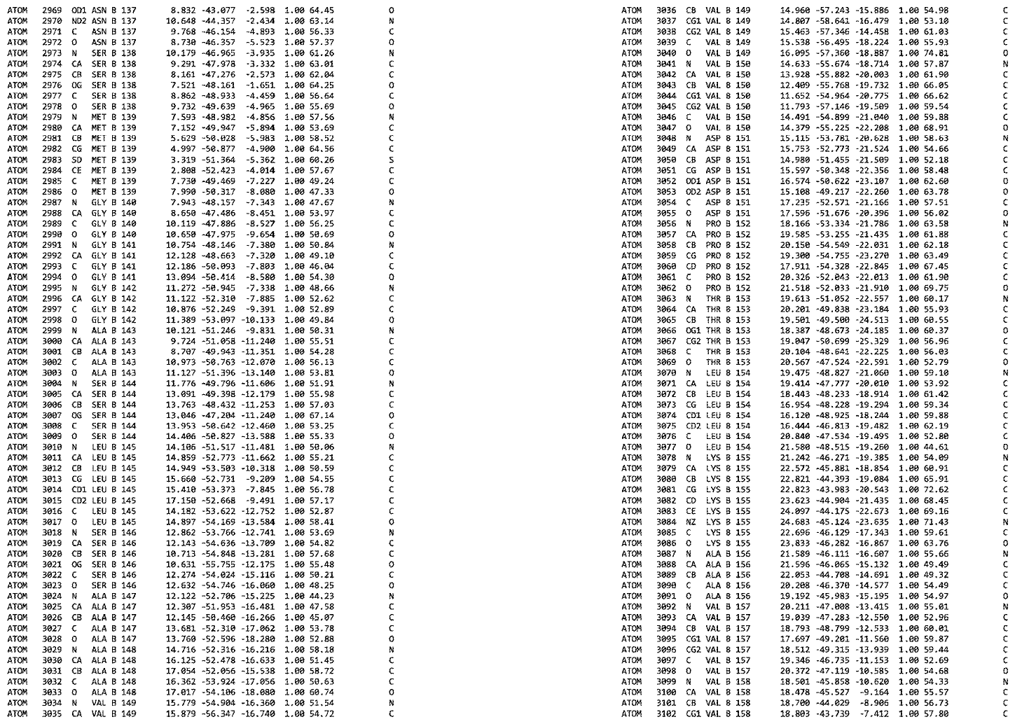

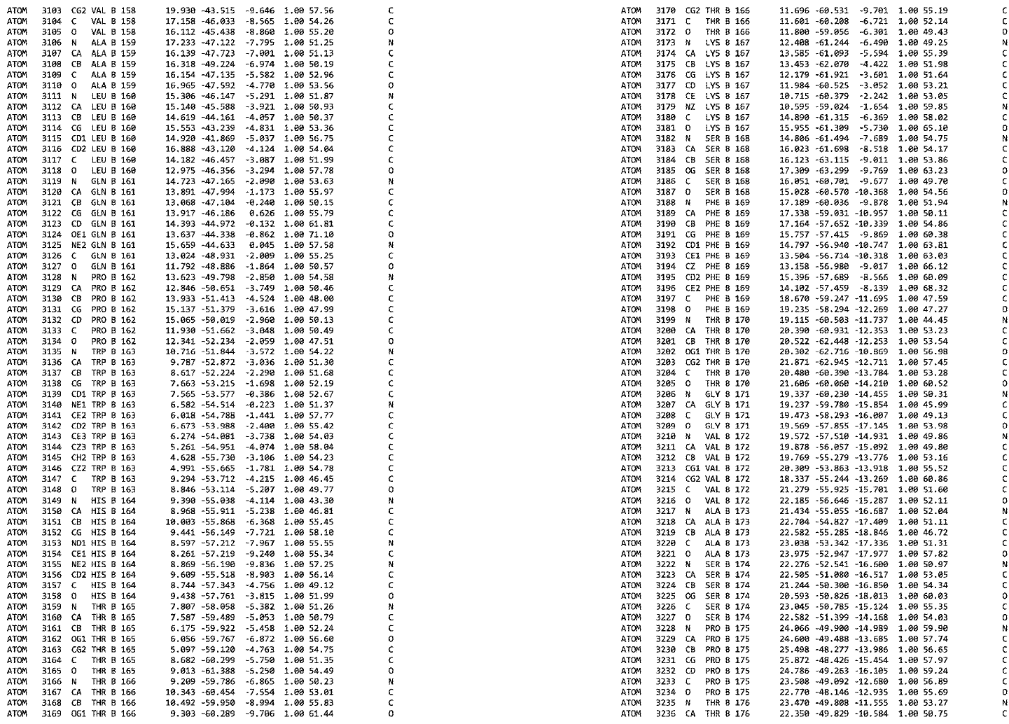

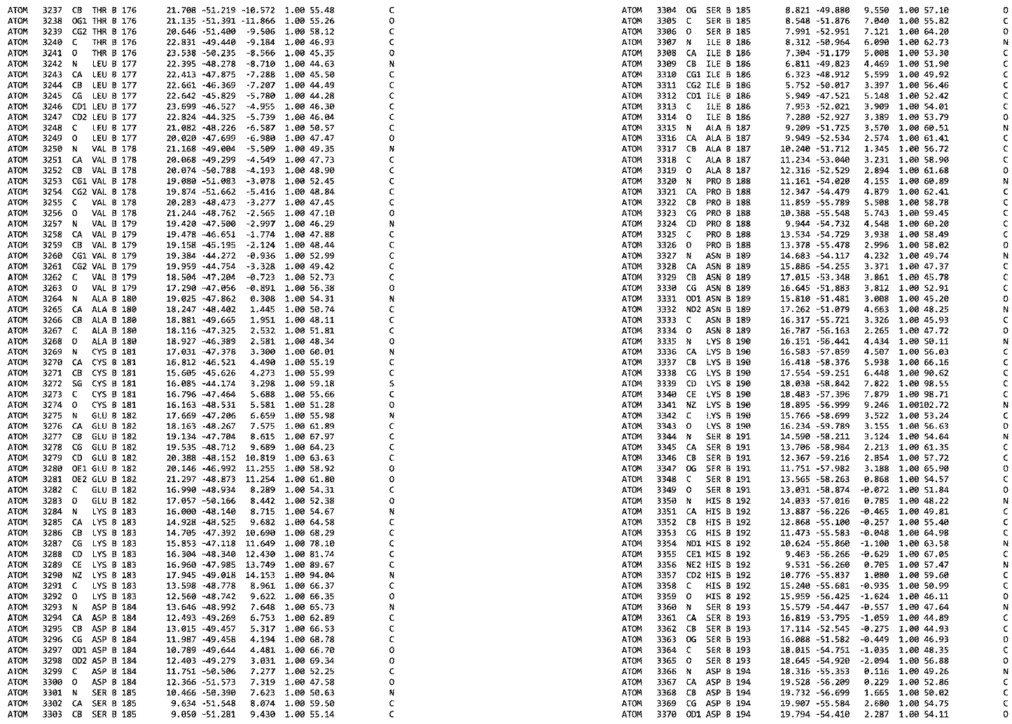

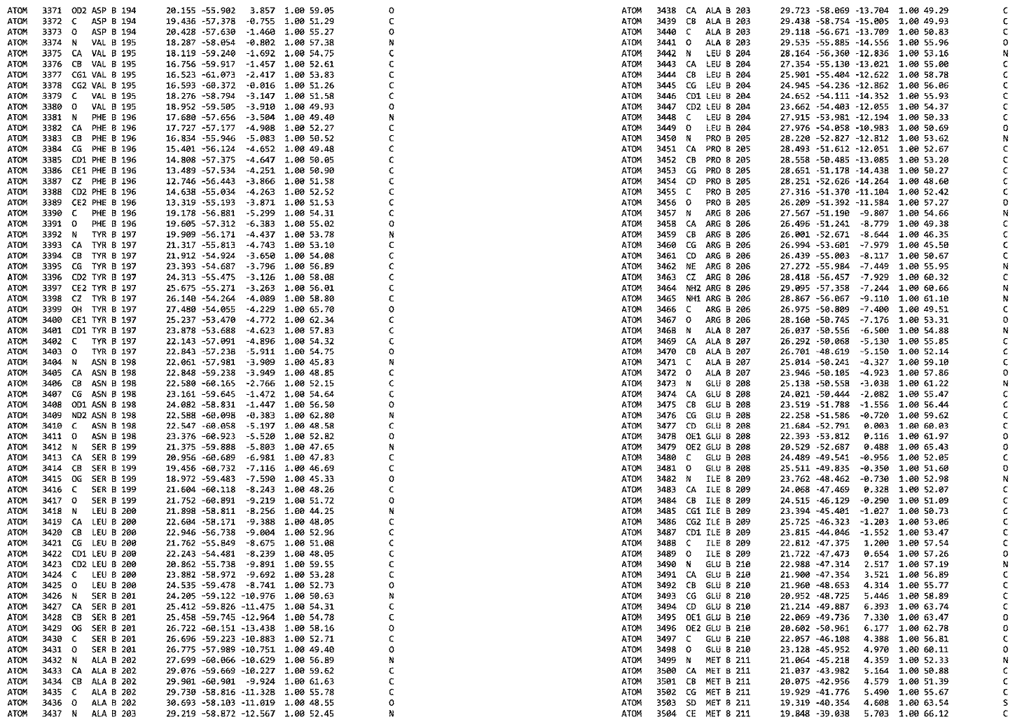

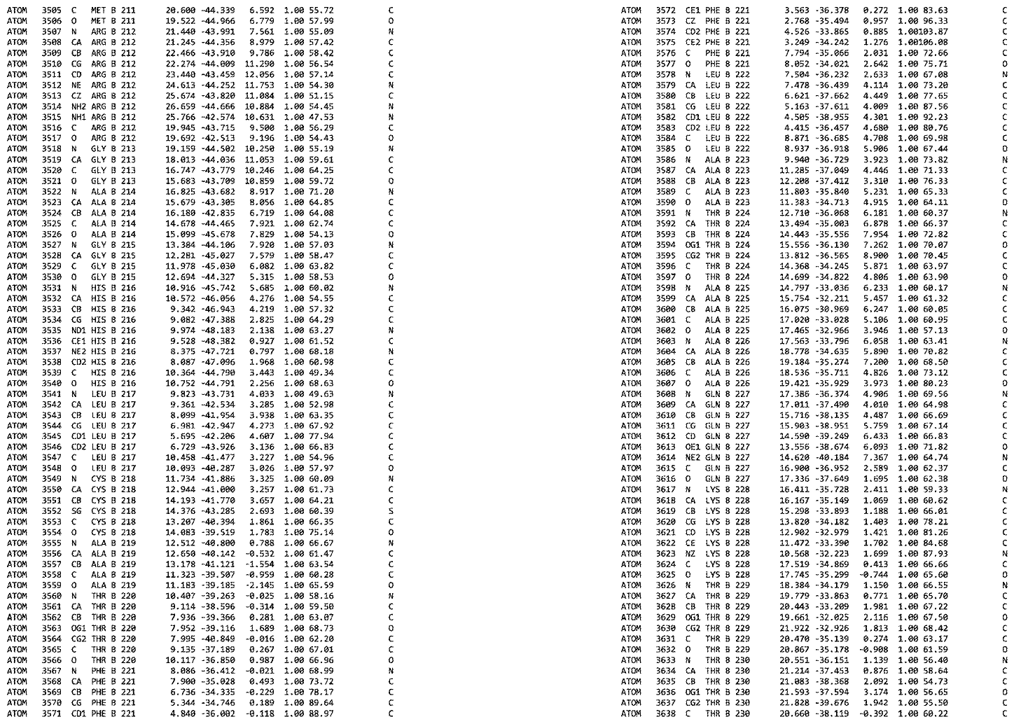

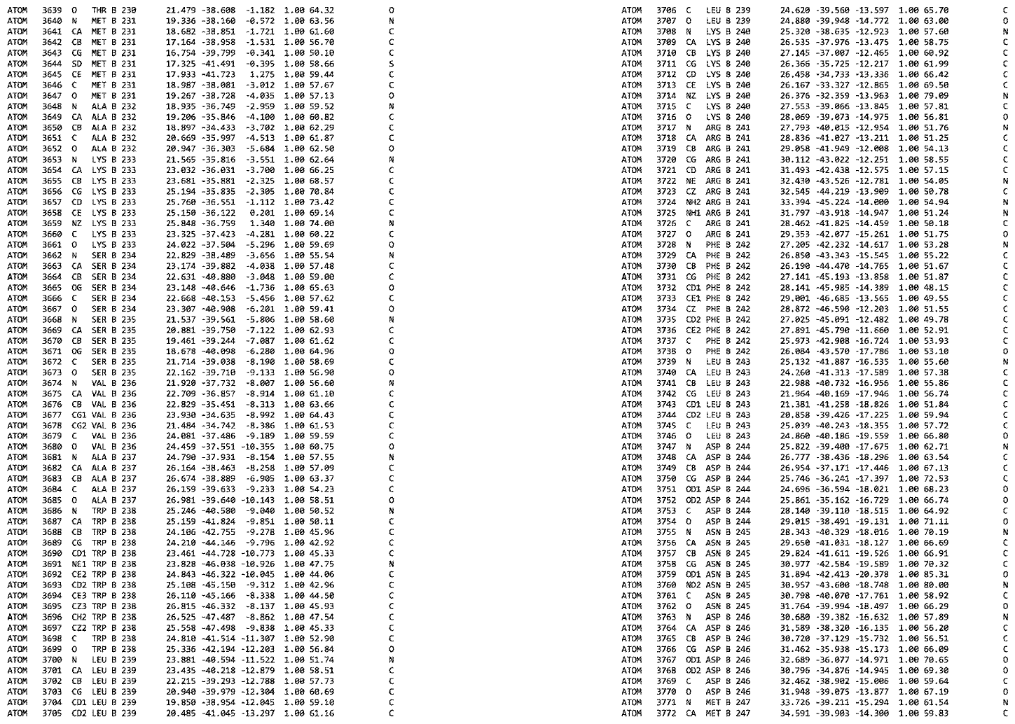

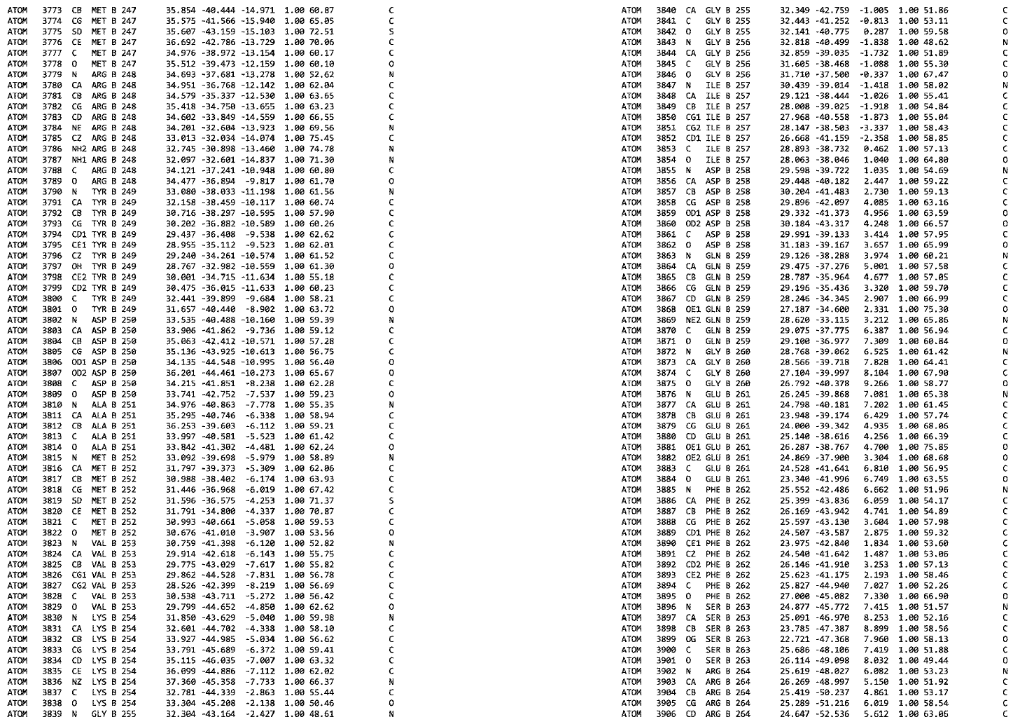

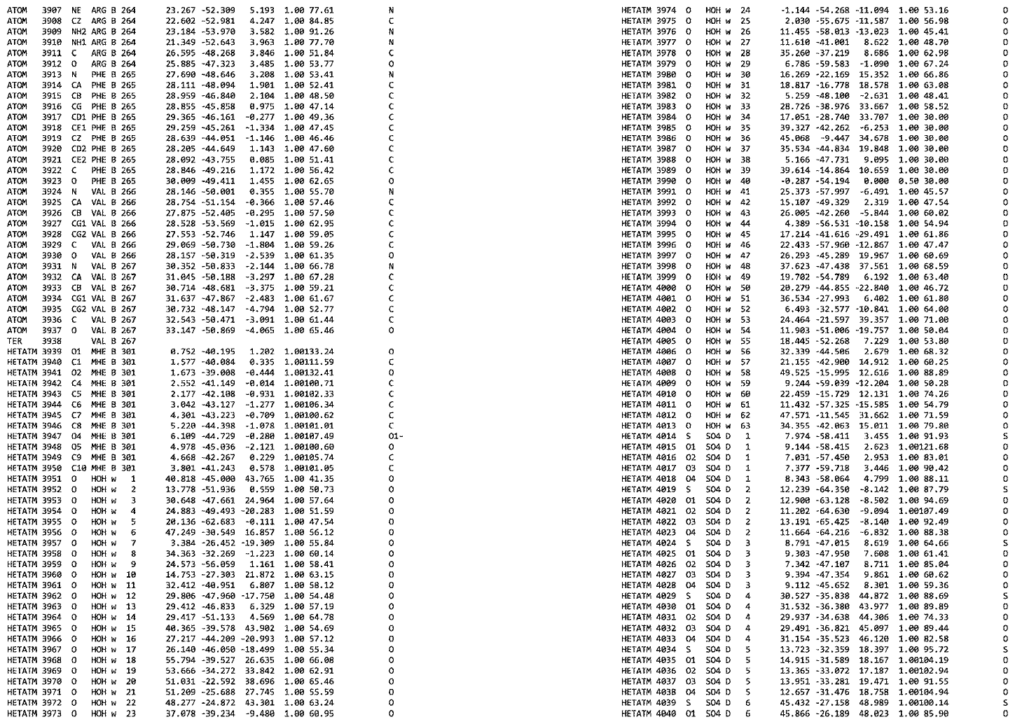

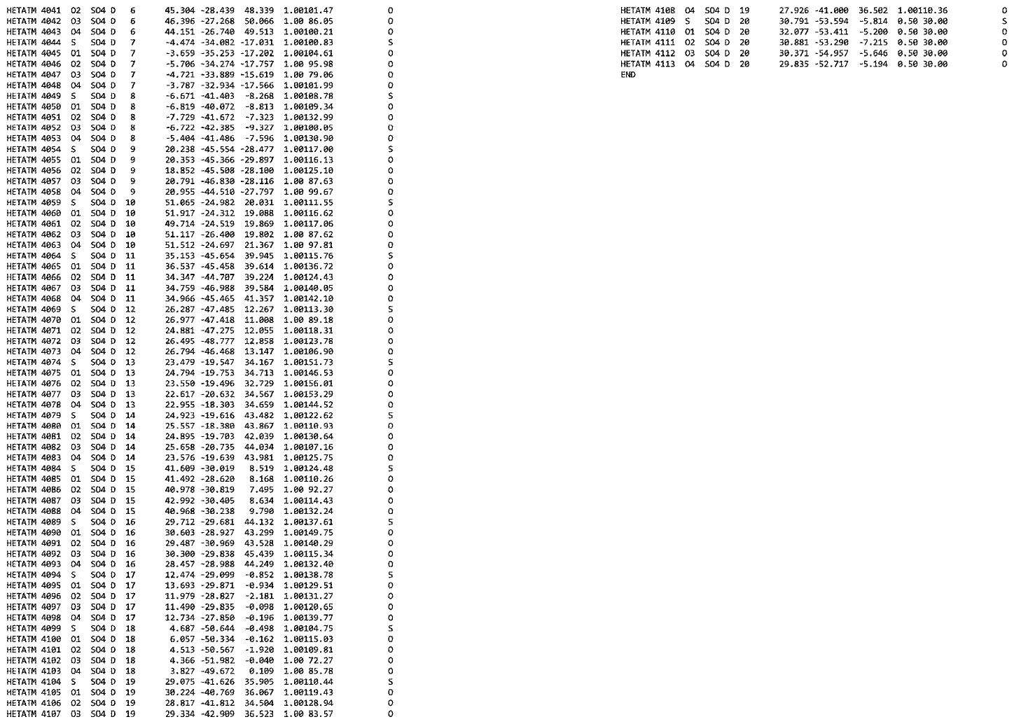

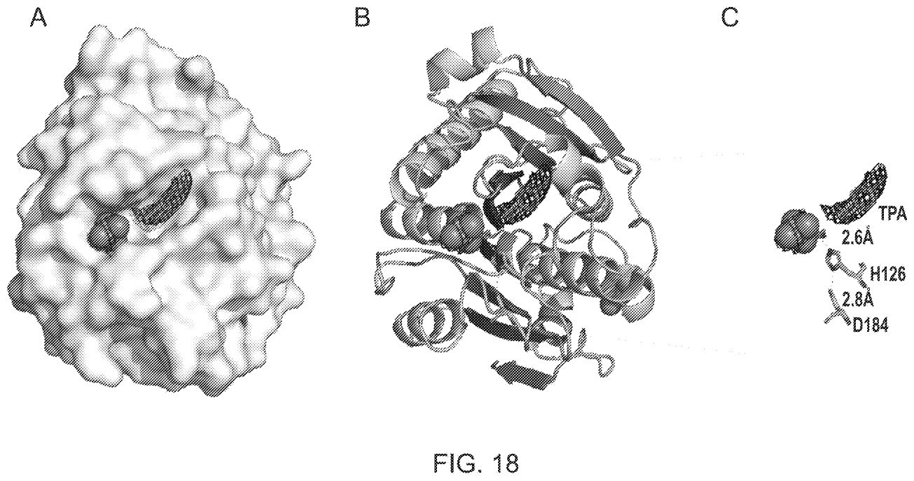

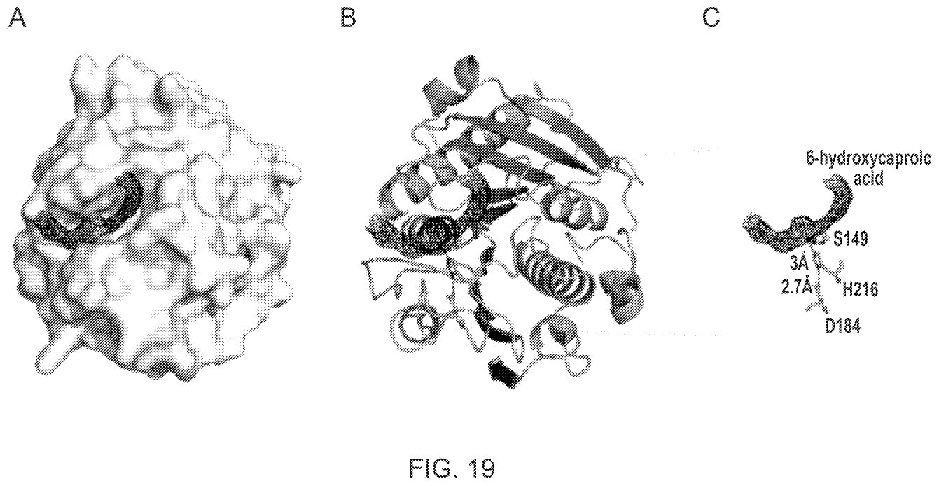

nylon nanoparticles are shown in FIG. 18.

### Example 4: Method for Selecting and/or Optimising PETase Activity Using Detection of Light Scattering From Plastic Nanoparticles

### Generation of Error-Prone-PCR Libraries

Two ep-PCR libraries of CI5En2, denominated as library 1 and 2, respectively, were generated using the GeneMorph II Random Mutagenesis Kit (Agilent, Cat N 200550) for the first round of directed evolution. The cl5en2 gene was amplified and randomly mutated by error-prone PCR following manufactures instruction. The ePCR reaction mix consisted of forward 5′ GGTACCGAAAACCTGTACTTCCAG 3′ and reverse 5′ CTGGGATTTAGGTGACACTATAGAATACTC 3′ primers (0.5 μM each), dNTPs (0.2 mM each), mutazyme II DNA polymerase (1 μL, 2.5 U/μl), 5 μL 10×Mutazyme II reaction buffer, and 3.5 μg and 699 ng of DNA template for the first and second library, respectively. The amplification occurred by using a PCR program [60 s 95° C., 25×(30 s 95° C., 30 s 59° C., 60 s 72° C.), 600 s 72° C., final hold 4° C.]. The resulting PCR products were purified using a Clean & Concentrator Kit-5 (Zymogen, Cat. N° D4003), quantified via nanodrop and then ligated into pExp-bla vector at BamHI and HindIII sites via slic cloning as previously described. The ligation products were purified again by using Clean & Concentrator Kit-5 and the obtained purified products were transformed into electrocompetent E. coli 10G Elite cells (Lucigen). The cloning was confirmed via DNA sequencing and mutation frequency was calculated. The transformation of 50 ng from each ep-PCR library into E. coli yielded 187 and 119 variants for 10⁻³ serial dilution of library 1 and library 2, respectively, on an LB agar plate (containing 100 mg/mL ampicillin) covering the library size 89 and 93.25 times for library 1 and 2, respectively.

### Preparation of Bacterial Suspensions for Droplet Encapsulation

The transformed bacteria were grown overnight at 37° C., then incubated at RT for 1 day. Colonies were subsequently scraped from agar plates and plasmids isolated using a DNA extraction Kit (GeneJet Plasmid Mini Kit,) following the manufacturer's instructions. 30 ng of extracted plasmid DNA were transformed into E. coli BL21 (DE3) cells and plated on LB agar (containing 100 mg/mL ampicillin) then incubated overnight at 37° C. and 1 day at RT. Colonies were gently scraped from the plate with LB media containing 100 mg/mL ampicillin and the obtained culture was incubated at 37° C. for three hours, after induced with IPTG for a final concentration of 0.4 μM and incubated at 20° C. overnight. The culture was centrifuged at 4000×g for 6 minutes and washed 3 times with buffer (100 mM Tris-HCl, 100 mM NaCl buffer, pH 8). The cell density was adjusted by dilution to obtain the required cell/droplet ratio after compartmentalization. Assuming a Poisson distribution for bacterial encapsulation, (Huebner Chem Commun 2007) a cell density OD600nm 0.01 should result in 20% of droplets with single cells for 150 pL droplets.

### Generation of Water-in-Oil Picolitre Droplets

Water-in-oil droplets (volume 150 pL) were generated using an 80 μm deep and 50 μm wide flow-focusing device with 25 μm nozzle bearing three inlets. Two inlets carry aqueous solutions prepared in GF buffer (50 mM Tris-HCl, 100 mM NaCl, 2.5% glycerol, pH 8.0). The streams from these inlets supplied (i) a cell suspension (OD₆₀₀ₙₘ depending on cell occupancy) and (ii) a mixture of the cell lysis reagents BugBuster (0.2% v/v, Novagen), Tween-80 (0.2% v/v, Merck), rlysozyme (96 U/mL; Merck) and plastic nanoparticles (˜1mg/mL). Fluorinated oil HFE-7500 (3 M) containing 008-FluoroSurfactant (2%, w/w, RAN Biotechnologies, USA) was injected through the third inlet. Aqueous solutions and the oil phase were injected using gas-tight syringes (Hamilton; 0.5, 1 or 2.5 ml) at a rate of 4, 4 and 32 μl min-1, respectively, with neMesys syringe pumps (Cetoni, Germany). Droplets were collected in the storage chamber made of an inverted 0.5 mL Eppendorf tube.

### Droplet Sorting and Electronics

Droplets were sorted according to the intensity of scattered light using a custom-built scatter activated droplet sorter (SADS), based on previously used system of fluorescent activated droplet sorter. Depending on the quality of the emulsion after the long-term incubation, droplets were injected into a sorting chip at 1-4 μL min-1 and spaced out with oil (2% w/w 008-FluoroSurfactant in HFE7500) at 20-50 μL min-1. As a result, droplets were sorted with the frequency of 100-350 Hz. It is important to note, that chemical composition of the spacing oil and the oil in the emulsion should be the same—otherwise the level of the signal might fluctuate along with various packing of the emulsion. We hypothesize that surfactant micelles present in the oil contribute to the level of the scattered light, thus it is useful to keep surfactant concertation stable during the sorting. Intensity of scattered light was measured by providing the incident light at λ=488 nm (Stratus laser, 488 nm, 25mW, Vortran, USA) and detecting the scattered light at the same wavelength nm using a photo multiplier tube (PMT, PM002, Thorlabs). Sorting was performed using a field-programmable gate array (FPGA, PCIe-7841R, National Instruments) which monitored and recorded the signal using custom LabVIEW software. The entire process was monitored on an inverted microscope (IX73, Olympus).

### DNA Recovery

Sorted droplets were collected into a 1.5 mL low DNA retention reaction tube (DNA LoBind, Eppendorf) and de-emulsified by adding 100 μL 1H,1H,2H,2H-perfluorooctanol (97%, Alfa Aesar). 100 μL of a 2 ng/μL salmon sperm DNA solution (catalogue number 15632-011, Invitrogen) was added to the collected droplets. The tube was vortexed briefly and centrifuged for 1 min at 1,000×g. The aqueous layer was transferred to a fresh reaction tube and the extraction was repeated twice. The aqueous phase was concentrated using a Clean & Concentrator Kit-5 (Zymogen) following the manufacturer's instructions. Elution was performed with 12 μL deionized water. 5 μL of the aqueous phase were used for PCR amplification [30 s 98° C., 25×(10 s 98° C., 30 s 55° C., 30 s 72° C.), 300 s 72° C., final hold 4° C.] using the pExp-F/R (for: ) primer pair. The PCR product was purified by agarose gel electrophoresis and recovered by using Clean & Concentrator Kit-5. The isolated DNA was subcloned into the pExp-bla plasmid via SLIC technique and the ligation product was again purified via Clean & Concentrator Kit-5. The purified products were transformed into electrocompetent E. cloni 10G cells.

### Secondary Screen

### Cell Lysis

Individual colonies of CI5En2 variants transformed into E. coli strain BL21 (DE3) were picked up and inoculated in 96 deepwell plates containing 1 mL of LB media with 100 μg mL-1 ampicillin. The cells were grown at 37° C., 200 rpm, for 3 hours and then 400 μM of IPTG was added to provide protein expression, which occurred at 20° C., 200 rpm, for 20 h. After expression, cells were harvesting by centrifugation at 4000×g for 45 minutes at 10° C. and the supernatant was discarded. To the cells were added 100 μL of lysis buffer [(100 mM Tris-HCl pH 8.0, 100 mM NaCl, 1× of BugBuster and lysonase (Merck Millipore, Cat. N° 71230-4)] and plates vortexed until complete cell resuspension, then incubated at 37° C. for 30 min to better provide cell lysis.

### Validation Via Hydrolysis of p-NP Acyl Esters

We also validate the kinetic parameters of CI5En2 variants by measuring the formation of p-nitrophenol after p-NP acyl esters hydrolysis. The reactions were measured forward an enzyme-free blank to subtract auto-hydrolysis and the released p-nitrophenol was monitored by spectro-photometrical quantification in SpectraMax® M3 Microplate Reader (Molecular Devices) at 405 nm (εpNP=18.300 M−1.cm−1). As substrate, we used p-NP acyl dodecanoate (C12) and p-NP acyl dodecanoate (C16) and the reaction was performed by using 10 μL of cell lysate and 90 μL of p-NP acyl esters at 1.11 mM. All assays were carried out in a buffer containing 50 mM Tris-HCl pH 8.0 and 0.3% triton X-100 (v/v) in 96-well microplates (ThermoFisher, Cat. N° 260836) with a final volume of 100 μL at 22° C.

### Validation of Hits Using PET NPs

The enzymatic hydrolysis of PET NPs using cell lysate was analysed by monitoring the change of turbidity of a suspension at 600 nm in microtiter plates. In each well 0.29 mg/mL PET nanoparticles made from cryo-milled powder were incubated with 0.5% Tween-80 and 10 μL cell lysate prepared as described above. The assay was carried out in a buffer containing 50 mM Tris-HCl, 100 mM NaCl and 2.5% glycerol at pH 8.0 with a final volume of 100 μL at RT. The change of turbidity was monitored over an incubation period of several days.

### Biochemical Characterization

To calculate the thermostability of CI5En2 wild-type, CI5En2 variants and IsPETase, the purified enzymes at 2 M were mixed with Sypro Orange protein gel stain (Invitrogen, Cat. N° S6650). The assay was performed in 50 mM Tris-HCl pH 8.0 and the denaturation of the enzymes was monitored in a CFX Connect Real-Time PCR Detection System (Bio-Rad) by increasing the temperature from 25 to 95° C., 0.5° C. every 30 s. The fluorescence of the SYPRO orange was measured (λ_{excitation}=410 nm, λₑₘᵢₛₛᵢₒₙ=610 nm) and the melting temperature (Tm) calculated using KaleidaGraph 4.1.3 (Synergy). The melting temperature was defined as the temperature where half the enzyme was denatured, and calculated as first derivative for each temperature-fluorescence curve.

### Results

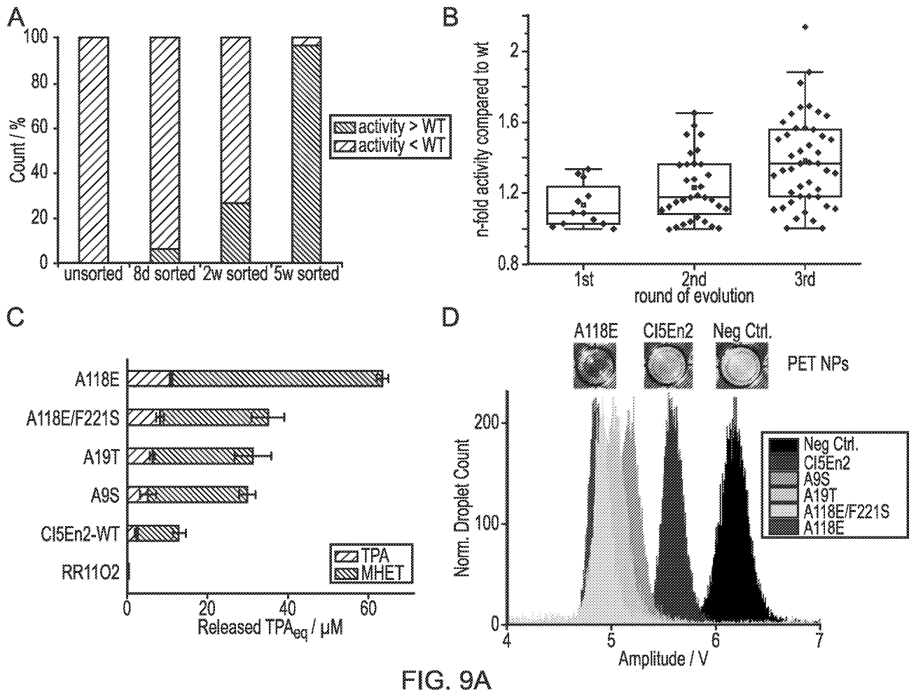

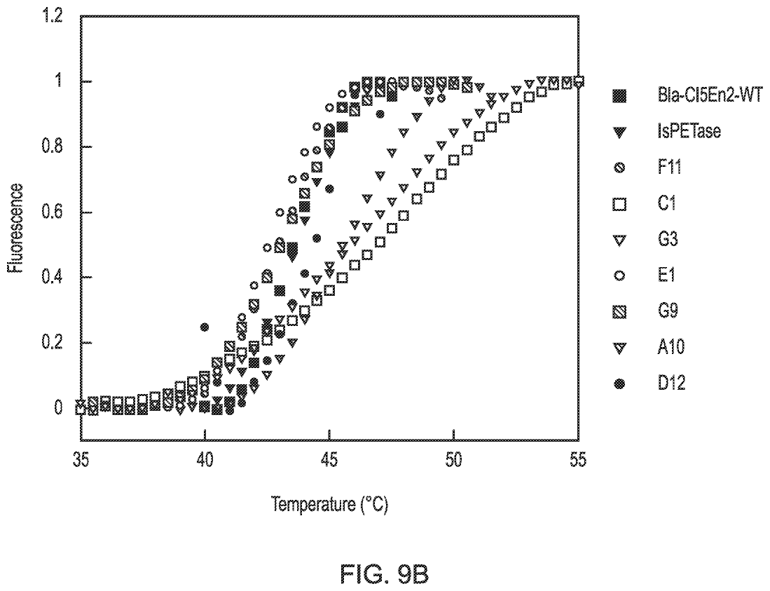

The results are shown in FIG. 9.

Cl5En2 improved on PET

| | | | Improvement |
|---|---|---|---|
| | Mutation | Corresponding | compared to Cl5En2- |
| Variants of Cl5En2 | nucleotide | mutation on aa | wt |
| Lib1-8d-P\|1 F11 | 25 GCAΔTCA | A9S | 2.5-fold on PET |
| | 162 GAAΔGAG | E54= | |
| Lib3-t4w-P\|2 G9 | 353 GCAΔGAA | A118E | 3.6-fold on PET |
| | 662 TTTΔTCT | F221S | |
| Lib2-t5w-P\|4 F9 | 21 GGCΔGGT | G7= | 5-fold on PET |
| | 353 GCAΔGAA | A118E | |
| 2^{nd} Shuf t1w-P\|1 G5 | 55 GCAΔACA | A19T | 2.5-fold on PET |

where = denotes a silent mutation.

Variants more thermostable than Cl5En2-wt

| | | | Improvement on Tm |
|---|---|---|---|
| | Mutation | Corresponding | (ºC) compared to |
| Variants of Cl5En2 | nucleotide | mutation on aa | Cl5En2-wt |
| Lib3-t4w-P\|1 D1 | 353 GCAΔGAA | A118E | +2.69º C. |
| | 662 TTTΔTCT | F221S | |
| | 703 AGCΔGGC | S235G | |
| Lib3-t4w-P\|2 G9 | 353 GCAΔGAA | A118E | +2.84º C. |
| | 662 TTTΔTCT | F221S | |
| Lib3-t4w-P\|2 E1 | 55 GCAΔACA | A19T | +3.20º C. |
| | 353 GCAΔGAA | A118E | |
| | 662 TTTΔTCT | F221S | |
| Lib3-t4w-P\|2 D12 | 66 TATΔTAC | Y22= | +2.94º C. |
| | 662 TTTΔTCT | F221S | |

### Example 5: Enzyme Identified From Example 1: RR11O2

Among the metagenomic hits identified here in this work is RR11O2. Although RR11O2 has been identified in a functional assay as an esterase it has a very low sequence conservation with other known esterases. The nearest sequence similarity is 38% with a hypothetical protein from a hydrocarbon metagenome (NCBI access number KUG22667.1).

Nucleic and amino acid sequence of RR11O2. RR11O2 was cloned into pHAT and pHAT3 vectors for expression and the full construction of RR11O2 pHAT is represented here. The nucleic and amino acid sequence of pHAT are highlighted (first 13 amino acids in grey) as well the nucleic acids of RR11O2 which were optimised for E. coli according to its codon usage (in red). The catalytic triad (S149, D311 and H308) of RR11O2 is also highlighted.

```

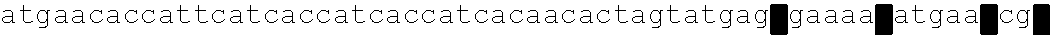

  M N T I H H H H H H N T S M S E N M N R

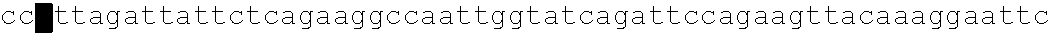

  P L D Y S Q K A N W Y Q I P E V T K E F
gacacattttatgtatatgcaaccgagtacatcctgagtagcatggaggagggtgctccg
  D T F Y V Y A T E Y I L S S M E E G A P
gattatgctgatatggaaaatgccgaaatgctggagggtgctgctgcagaatatatgttg
  D Y A D M E N A E M L E G A A A E Y M L
cacgccaccgcatacgcagattccaccaacgtgttcatgccttactaccgtcaagtaggc
  H A T A Y A D S T N V F M P Y Y R Q V G
ttgcgttatgccggggtcgtctggaagagggacggaatcttcgatgcctccgttgctggt
  L R Y A G V V W K R D G I F D A S V A G
atgccttatggcgacattgttgccgcattagactattacttcgagcactacaacaacggc
  M P Y G D I V A A L D Y Y F E H Y N N G

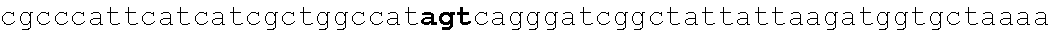

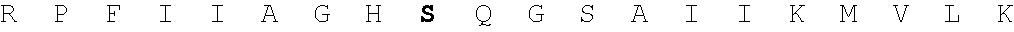


aaatattttgtggagcaccccgattactacaagcgtatgattgccgcttacccgattggc
  K Y F V E H P D Y Y K R M I A A Y P I G
tacgctttcacgaaggacgagttcaagacatacccacatatgaagtttgccactggtgag
  Y A F T K D E F K T Y P H M K F A T G E
tgcgatacgggtgttatcatcacctggaataccgaaggacctaagaacagggaagtaaac
  C D T G V I I T W N T E G P K N R E V N
gctgatacgtgtgtgctgcagccgggttctatgagtataaacccgcttaactggaaactg
  A D T C V L Q P G S M S I N P L N W K L
gacgacacttatgctccggccagcatgaatctggggtctctttttccgaacaaagatacc
  D D T Y A P A S M N L G S L F P N K D T
ggcaaactggagattcaggacctgggtgcggacgcacaggtgttccctgaccgcggagtg
  G K L E I Q D L G A D A Q V F P D R G V
gtagttacccatgccatgggtgaggaaatgaccgaggagaccgccaaagtggcagccgaa
  V V T H A M G E E M T E E T A K V A A E

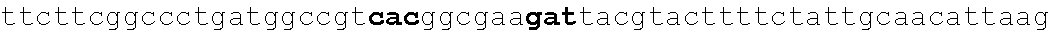

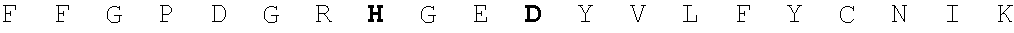


gataatgtggctaaacgtatctcggcctatatggcaaaccgcaagtag
  D N V A K R I S A Y M A N R K —
```

The enzyme activity of purified RR11O2, Bla-CI5En2 and IsPETase was tested against different p-NP acyl esters [acetate (C2), butyrate (C4), hexanoate (C6), octanoate (C8), decanoate (C10), dodecanoate (C12) and myristate (C14)]. The final enzyme concentration of the enzymes in the reaction depended on the p-NP acyl ester used as substrate. Kinetics were measured at enzyme concentrations of RR11O2, Bla-CI5En2 and IsPETase between 31.25 μM to 23.2 μM, 2.5 nM to 50 nM, and 50 nM to 4.8 μM, respectively. The p-NP acyl ester substrates were used in a concentration range between 0 and 4 mM. The kinetic parameters Vmax and Km were calculated using Kaleidagraph by non-linear lest square fits of initial rates to the Michaelis-Menten equation. The catalytic rate constant k_{cat} (mM-1) was calculated from the initial steady-state velocity according to the equation k_{cat}=Vmax/[E] and all enzymatic assays were conducted at 22° C. in a final volume of 100 μL, in a buffer containing 50 mM Tris-HCl pH8.0 and 0.3% Triton-X 100. The released p-nitrophenol was monitored spectro-photometrically at 405 nm (εpNP=18.300 M−1.cm−1) by taking time points in 20 second intervals in a SpectraMax® M3 Microplate Reader (Molecular Devices).

### Results

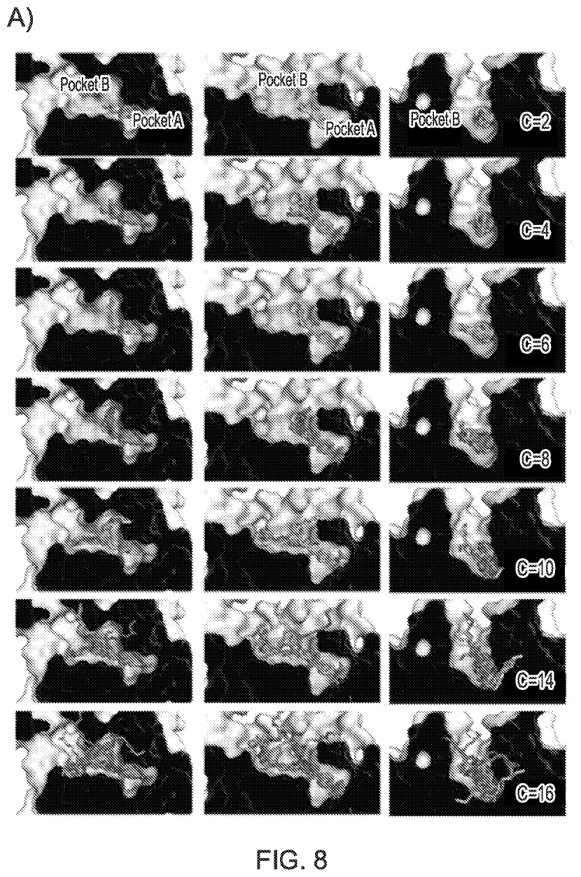

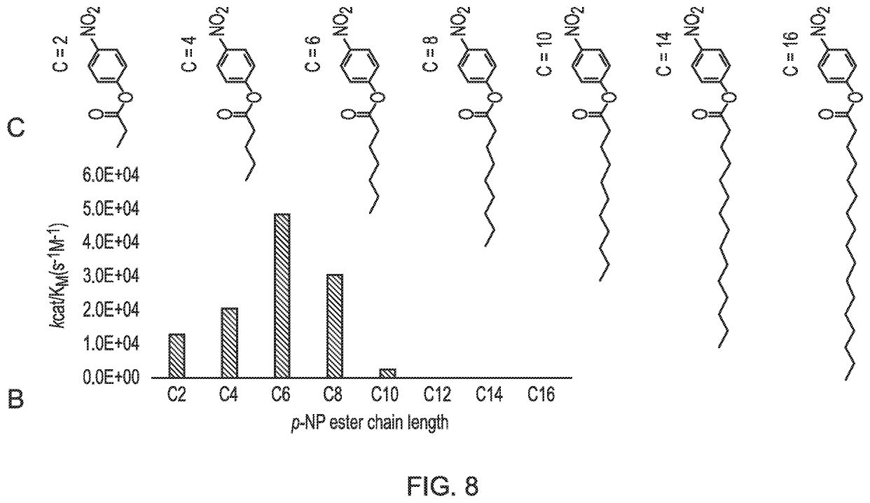

The results are shown in FIG. 8.

RR11O2 also exhibits promiscuous activity on PET.

### Example 7: Enzymatic Characterization of Enzymes

To detect the release of TPA, MHET and BHET in the presence of various enzymes, aliquots were taken from reaction mixtures, turnover was stopped by addition of an equal volume of methanol containing 0.5% formic acid, centrifuged (16,100×g, 15 min) and analysed by HPLC (1260 Infinity II, Agilent; Nucleodur C18 analytical EC standard column (5 μm, 4×125 mm, Macherey-Nagel, Germany). As the mobile phase mixtures of buffer A (0.1% formic acid in distilled water) and buffer C (distilled water), as well as buffer D (acetonitrile) were used at a flow rate of 1 mL/min. Buffer A was kept at 20% for the whole run. The mobile phase was changed gradually after 5 min from 72% C and 8% D to 50% C and 30% D until 13 min and then to 30% C and 50% D until 17 min before it changed to 10% C and 70% D at 18 min. The latter conditions were kept until minute 25 and the changed back to the starting conditions of 72% C and 8% D until 30 min to initiate another run (see diagram below). TPA, MHET and BHET were detected at 250 nm by comparison with authentic samples and quantified by comparison to calibration curves.

### Scanning Electron Microscopy (SEM) to Analyse PET Film Morphology

SEM on the changes in the surface structure of treated PET films was observed using a Tescan MIRA3 FEG-SEM (Brno, Czech Republic) using the SE detector. The working voltage and distance were 5 kV and 6 mm, respectively. After incubation with 18 μM enzyme in 100 mM Tris-HCl and 100 mM NaCl buffer (pH 8.0) for up to one month, the PET samples were washed with 1% SDS solution, deionized water, ethanol and air dried. PET samples were mounted on aluminum stubs using carbon tape sputter-coated with 10 nm platinum. Electron micrographs of PET films were recorded by SEM at the desired magnifications.

### Weight Loss of PCL and PET Films

PCL (approximately 24 mg) and PET films (approximately 15 mg) were incubated with 1 or 2 μM enzyme in 500 μL 100 mM Tris-HCl, 100 mM NaCl buffer (pH 8.0). Hydrolysis was performed at 37° C. and 300 rpm for up to one week. A control was also performed by using only buffer solution without the presence of the enzyme. After treatment, PCL films were thoroughly washed three times with deionized water, dried until a constant weight was obtained. The plastic degrading activity were evaluated by measuring the weight loss of PCL and PET films, respectively. The weight loss of plastic films (%) was calculated by Eq. (1): Weight loss (%)=100×(W_(pre-degraded film)−W_(post-degraded film))/W_ (pre-degraded film) where W is the weight of PCL or PET films.

### Enzyme Assays for PET Nanoparticles and Film

Amorphous or crystalline PET nanoparticles (approximately 0.3 mg of dried powder) or self-made crystalline PET films (approximately 10-20 mg) were incubated with 6 μM purified and TEV cleaved proteins in Tris-HCl and 100 mM NaCl buffer (pH 8) at 37° C. The reactions were terminated at different time points (t0, t2h and t12h for amorphous particles and t0, t1d and t3d for crystalline particles) by adding an equal volume of methanol containing 0.5% formic acid. The supernatant obtained by centrifugation (16,100×g, 15 min) was analysed by HPLC.

### Enzymatic Hydrolysis of Polyester Nanoparticles in Microplates and in Microdroplets

The enzymatic hydrolysis of polyesters was analysed by monitoring the change of turbidity of a polyester nanoparticle suspension at 600 nm. The enzymatic hydrolysis was performed in microtiter plates (Nunc, Thermo Fisher USA) at RT with 0.23 mg/ml of PCL nanoparticles and 0.29 mg/ml of PET nanoparticles made from cryo-milled powder in each well. The change of turbidity was monitored over an incubation period of several days.

We also tested the activity of pure enzymes using microdroplets and scatter light measurements. The samples were prepared prior to encapsulation by mixing the nanoparticle with enzyme solutions. Plastic nanoparticles of desired density ranging from 0.5 to 2 mg/mL were stabilized in buffer (50 mM Tris-HCl, 100 mM NaCl, 2.5% glycerol, pH 8) using 0.1-0.5% (v/v) Tween-80 before addition of purified and TEV cleaved enzymes (500 nM to 2 μM). Afterwards the solution was split into two equal volumes, which were injected to the droplet generation chip using both aqueous inlets. The flow rates were the same as for generation of droplets with single

### Results

The results are shown in FIG. 5C.

### Example 8: Structural Characterization of RR11O2

However, the enzyme clearly shows esterase activity so understanding from the crystal structure how the function is conserved without conservation of the sequence is important.

Initially, truncated constructions of RR11O2 were designed in order to have different lengths and flexibility of N-terminal for protein crystallization.

Data was collected at the Diamond lightsource on beamline 103 and data from Diamond automated data processing pipeline with dials were used for the structure determination. Structure determination and refinement were performed using software from the CCP4 package and Phenix. Data collection and refinement statistics are shown in Table 6. Coordinates have been deposited to the Protein Data Bank (PDB) under accession number PDB 6ZZV.

Four truncated constructions were designed (M1_L9del, M1_N16del, M1_F27del and M1_Y33del). B-SDS-PAGE of solubility test of full-length RR11O2 cloned into pHAT3 and the four truncated constructions M1_L9del, M1_N16del, M1_F27del and M1_Y33del) cloned into pHAT. All constructions were grown at 37° C. and induced with 0.4 mM IPTG at 20° C. or 37° C. The abbreviation refers to M: Protein Ladder (Thermofisher Scientific, catalog number 26619), TO: without induction, TON: after overnight induction, S: soluble fraction, I: insoluble fraction. C-Melting temperature of Δ10D in 100 mM citrate pH 4.0, 200 mM NaCl and 10% glycerol (−); 100 mM Tris pH 8.0 and 100 mM NaCl (x); and 100 mM hepes pH 7.0 and 100 mM sodium formate (⋅). D-Crystallysation of M1_L9del on condition JCSG well F2 [0.1 M sodium citrate pH5.0, 3.2 M (NH4)2SO4] after 8 and 21 days incubation at 18° C., abbreviation: bright-field (BF) and ultraviolet light (UV). The obtained crystals (upper part of D) were submitted to the beamline and the diffraction pattern at 1 Å (down part of D) further processed.

TABLE 6

| Data collection Date | 26 Nov. 2018 | |
|---|---|---|
| Beamline | DIAMOND BEAMLINE I03 | |
| Deposition Session | D_1292110358 | |
| PDB | 6ZZV | |
| Data collection | RR11O2 | |
| Wavelength | | |
| Resolution range | 47.45-1.06 | (1.09-1.06) |
| Space group | P 1 21 1 | |
| Unit cell | 60.58 53.31 100.58 90 100.3 90 | |
| Total reflections | 1572595 | (38030) |
| Unique reflections | 267487 | (11653) |
| Multiplicity | 5.9 | (3.3) |
| Completeness (%) | 93.9 | (55.7) |
| Mean I/sigma(I) | 17.10 | (1.6) |
| Wilson B-factor | 10.8 | |
| R-merge | 0.044 | (0.744) |
| R-meas | 0.052 | (1.015) |
| R-pim | 0.029 | (0.685) |
| CC½ | 0.999 | (0.346) |
| Refinement | | |
| Resolution range | 47.45-1.06 | (1.09-1.06) |
| Reflections used in refinement | 267462 | (16632) |
| Reflections used for R-free | 13469 | (879) |
| R-work | 0.1646 | (0.3098) |
| R-free | 0.1723 | (0.3064) |
| CC(work) | 0.964 | (0.744) |
| CC(free) | 0.966 | (0.736) |

| | |
|---|---|
| Number of non-hydrogen atoms | 5733 |
| macromolecules | 5093 |
| ligands | 59 |
| solvent | 581 |
| Protein residues | 619 |
| RMS(bonds) | 0.006 |
| RMS(angles) | 0.98 |
| Ramachandran favored (%) | 98.04 |
| Ramachandran allowed (%) | 1.96 |
| Ramachandran outliers (%) | 0 |
| Rotamer outliers (%) | 0.38 |
| Clashscore | 1.19 |
| Average B-factor | 16.13 |
| macromolecules | 14.76 |
| ligands | 36.83 |
| solvent | 26.04 |

The modelling of the predicted binding mode of substrates to RR11O2 was done using the GOLD software package (CCDC) using the structure deposited in the PDB (PDB code 6ZZV) as the target. Substrate structures were generated using Grade web server. The site of interest was defined as a 20 Å sphere around Ser149. Ligand files were generated with grade (Global Phasing). Initial studies with p-NP hexanoate (C6) indicated that in order for a productive binding mode to be produced the aromatic ring would have to bind in pocket A (productive binding mode =ester binds within 3 Å of Ser149). Therefore, a pharmacophore constraint was introduced to place the aromatic ring of the substrates in Pocket A in order to study productive binding modes.

### Results

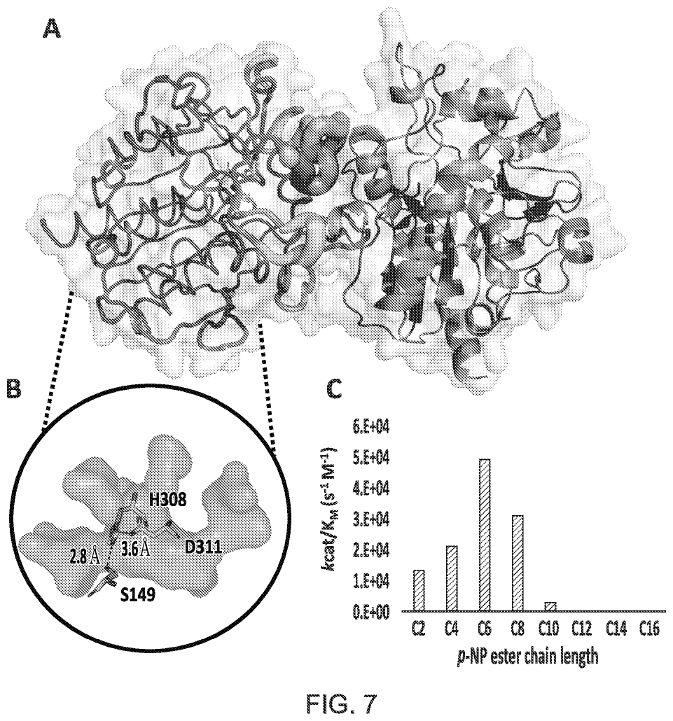

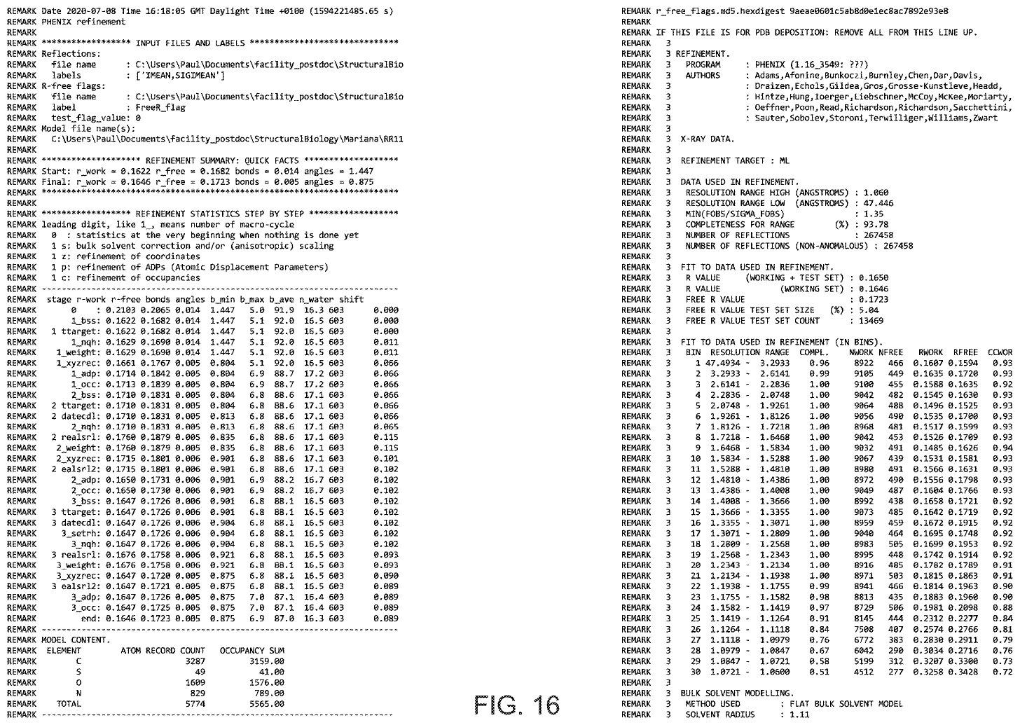

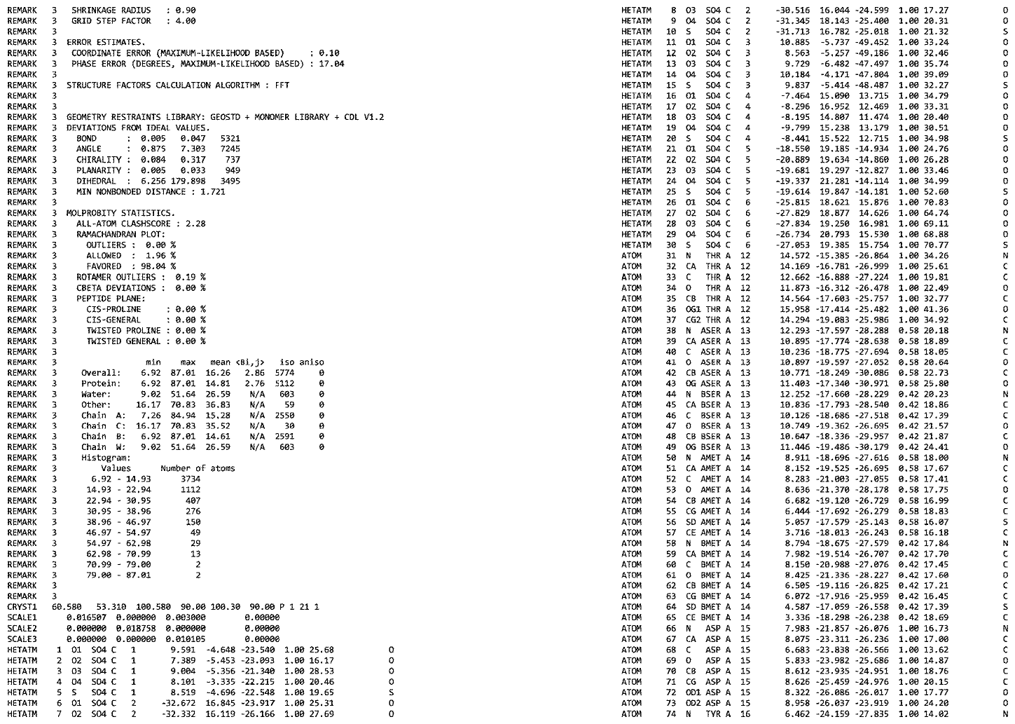

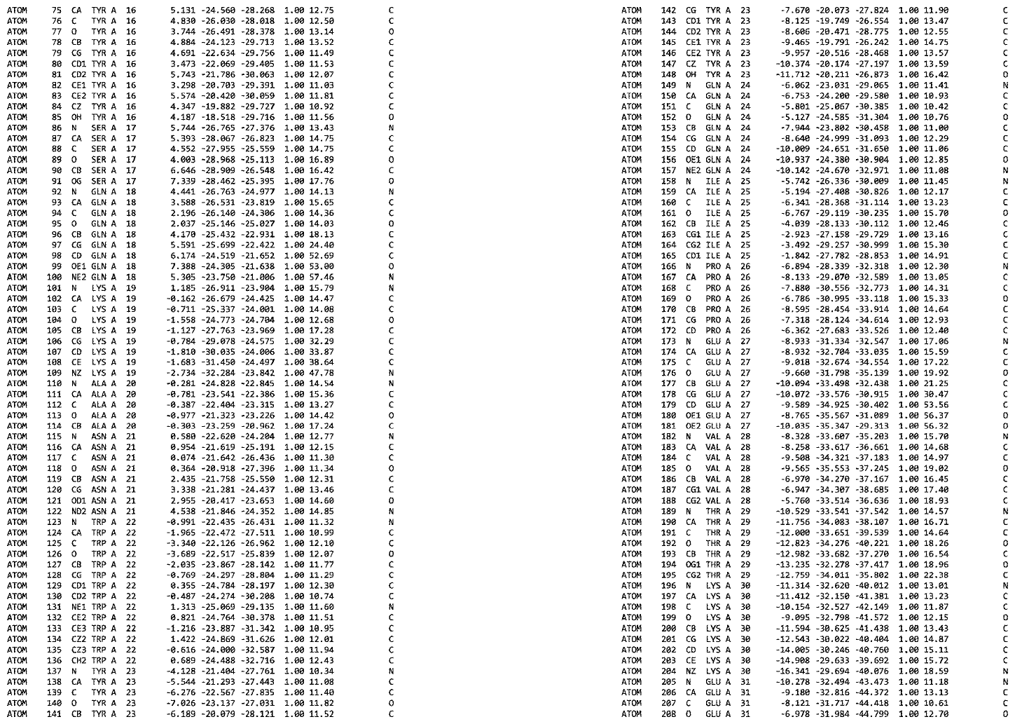

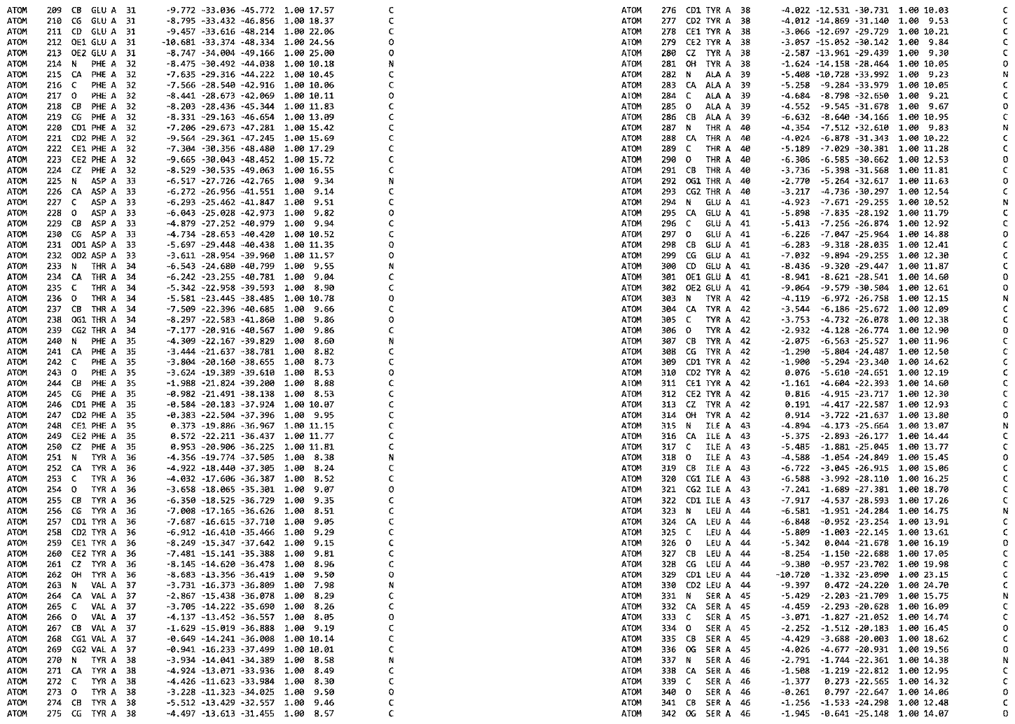

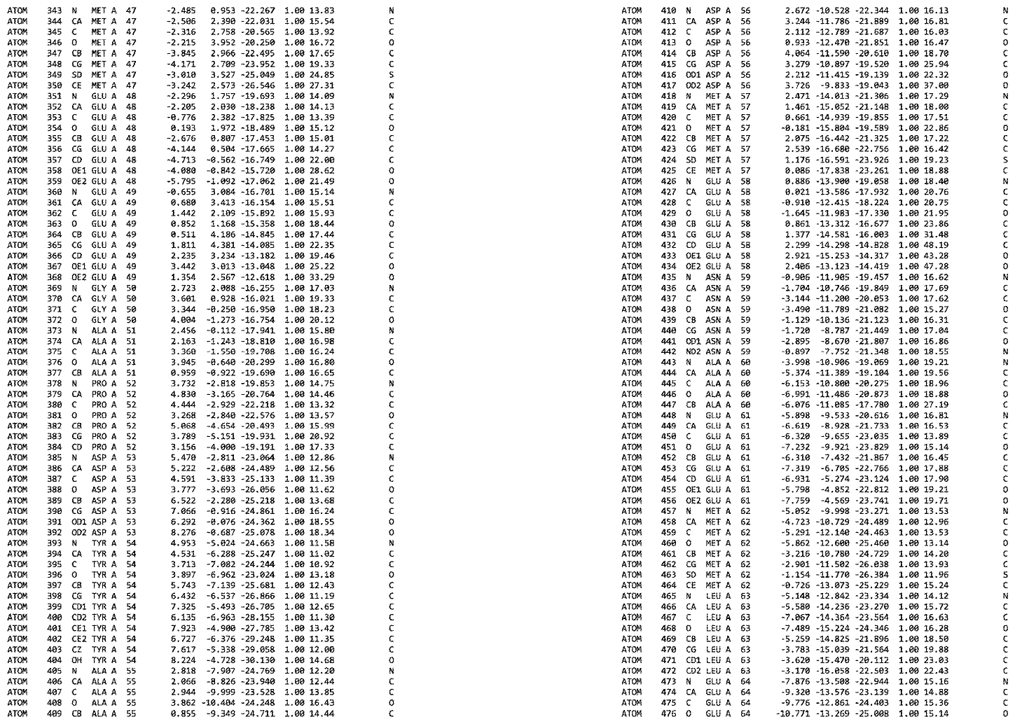

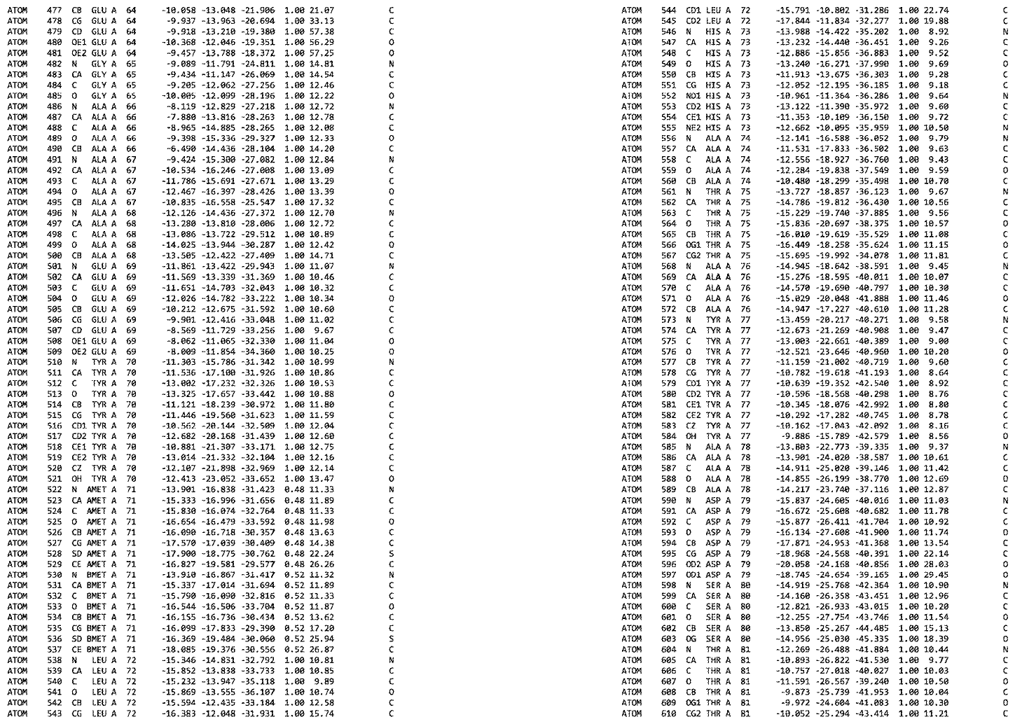

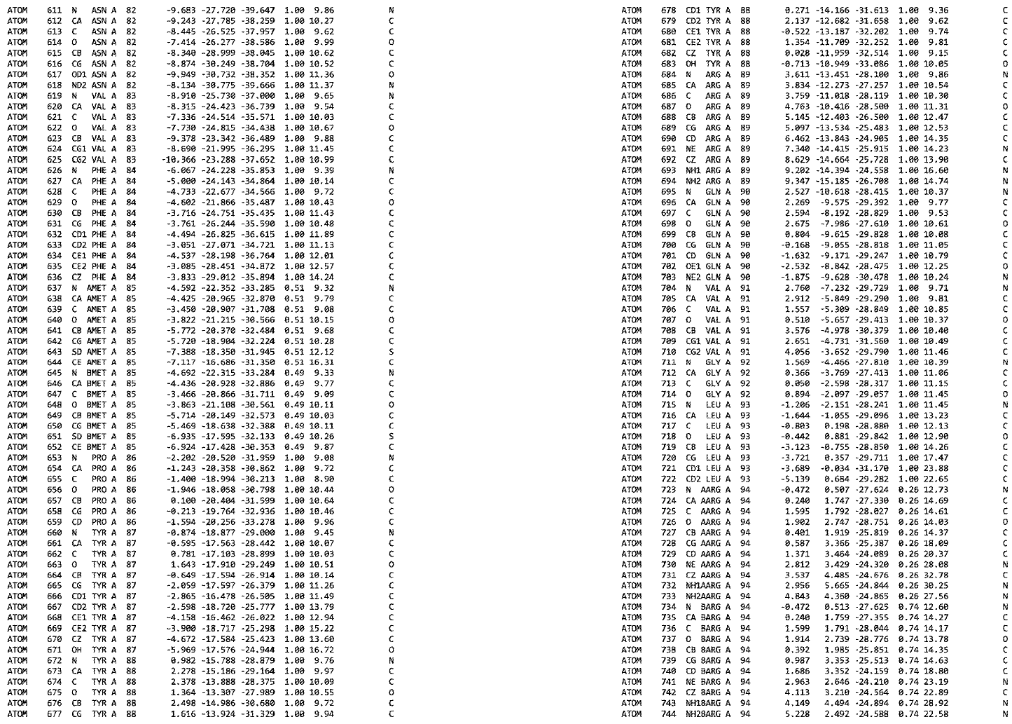

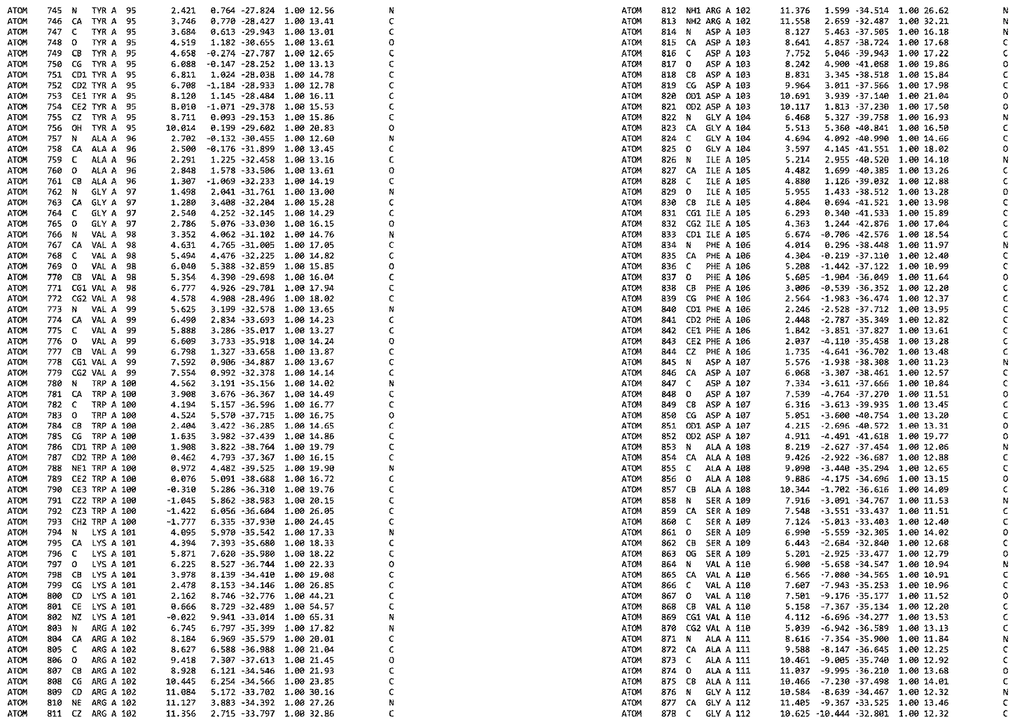

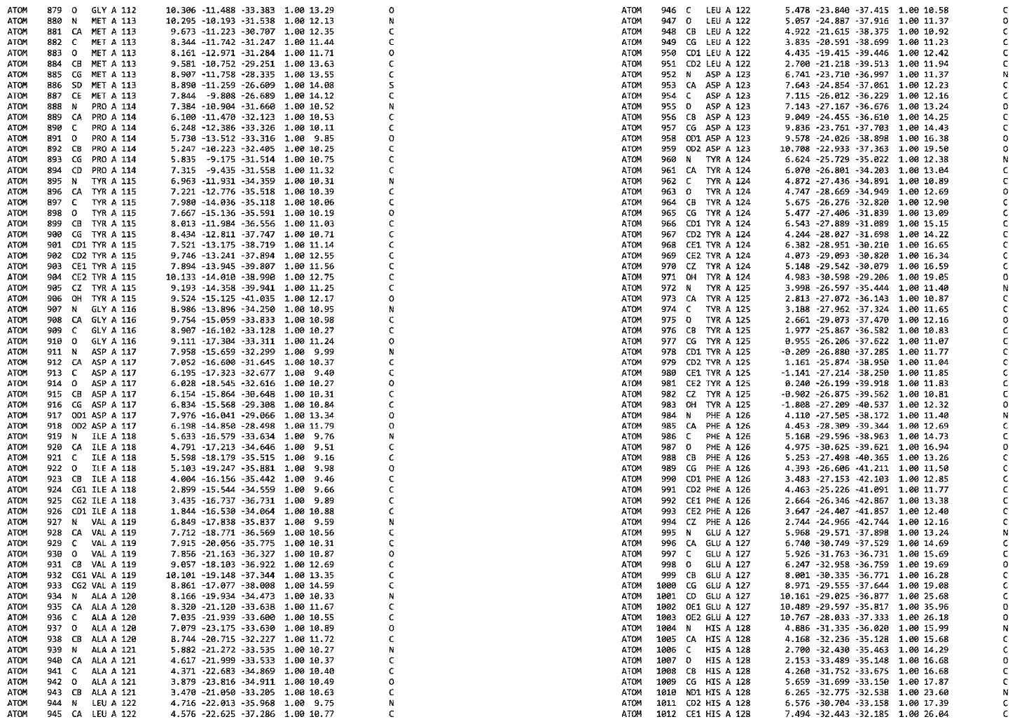

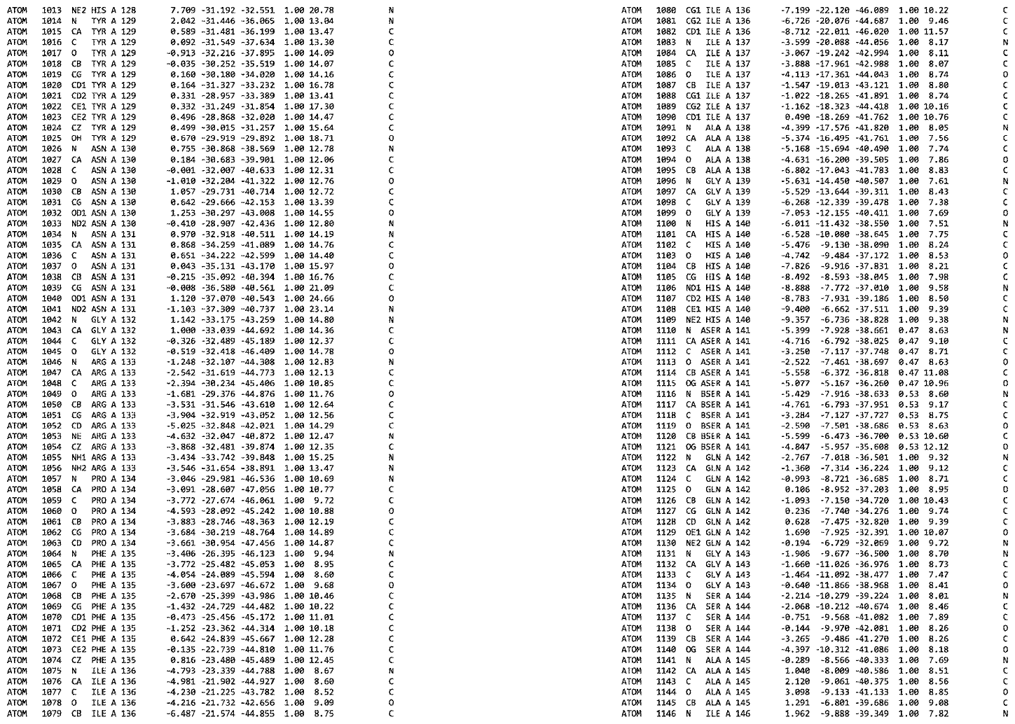

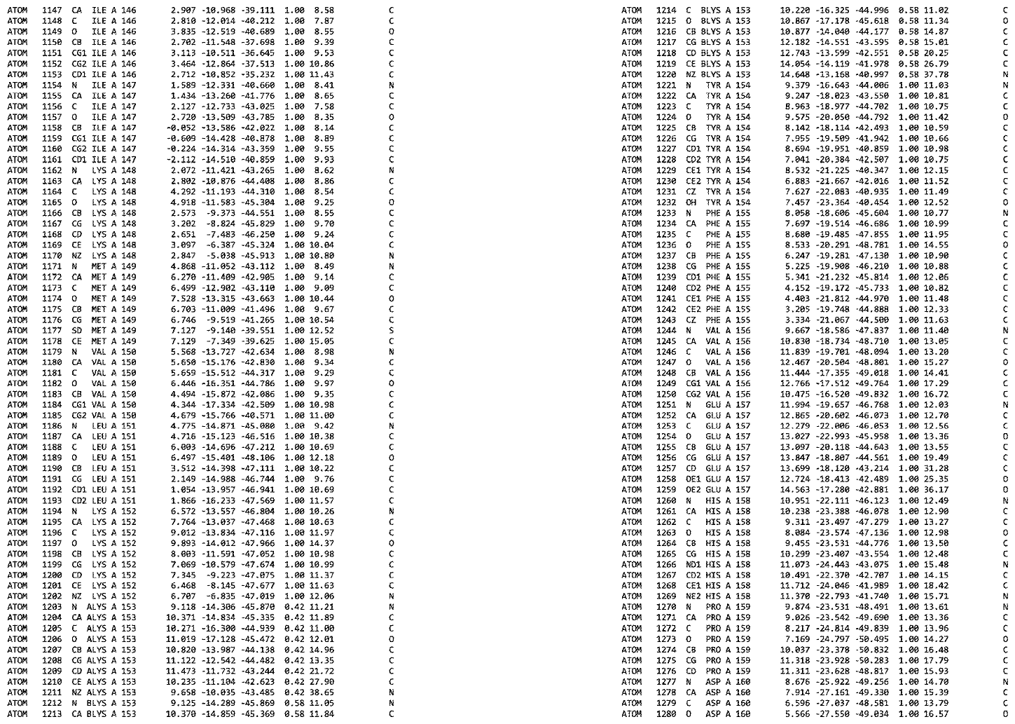

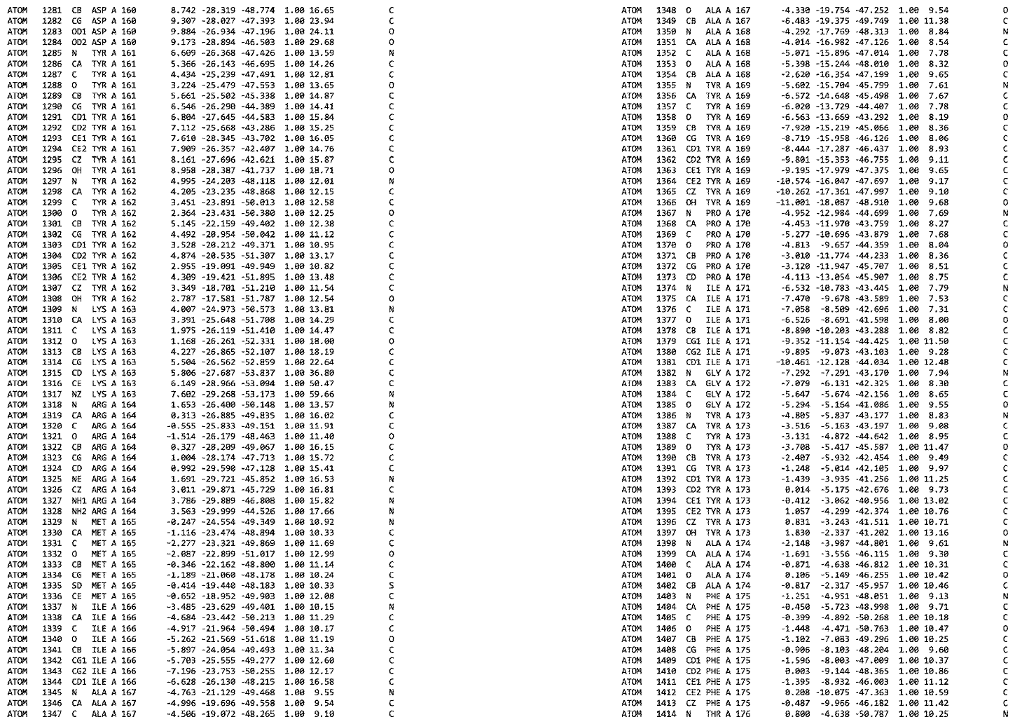

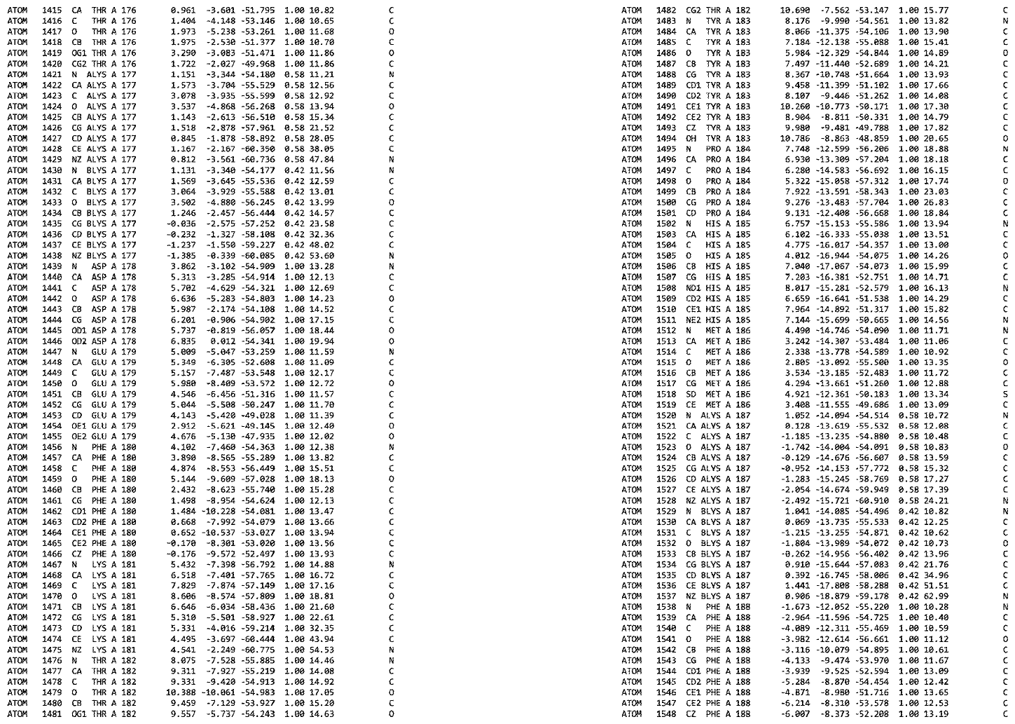

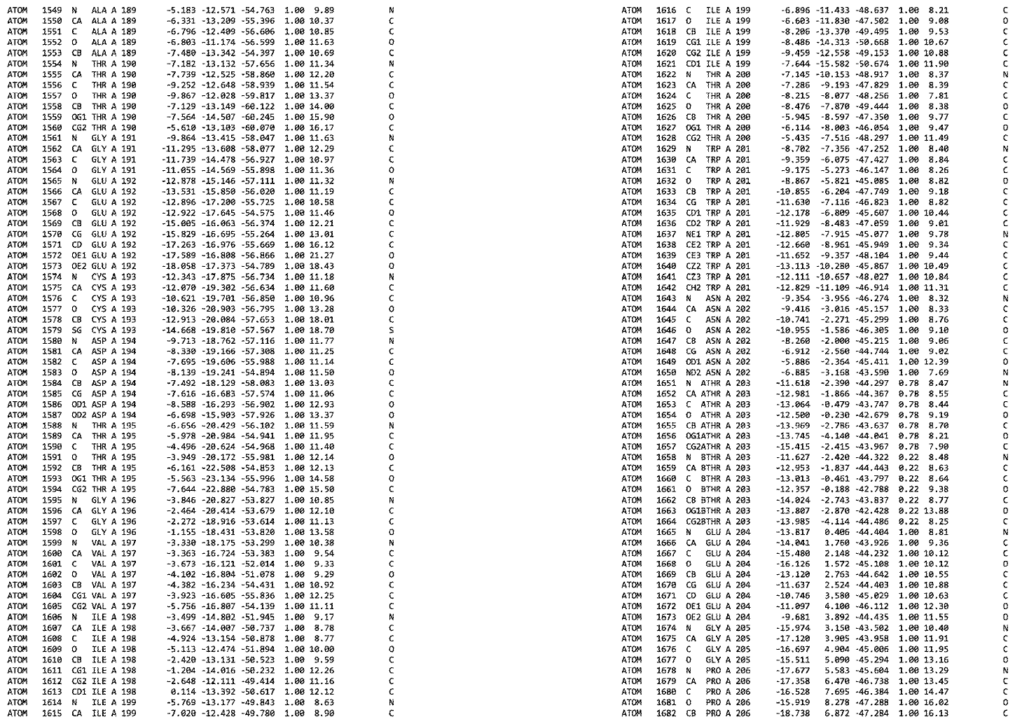

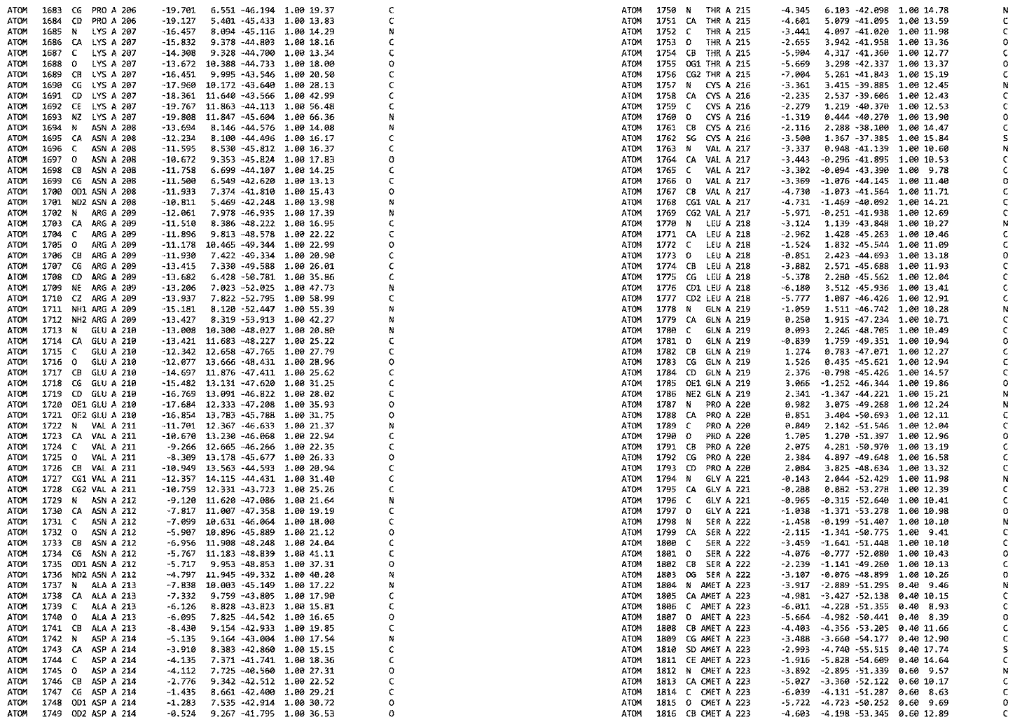

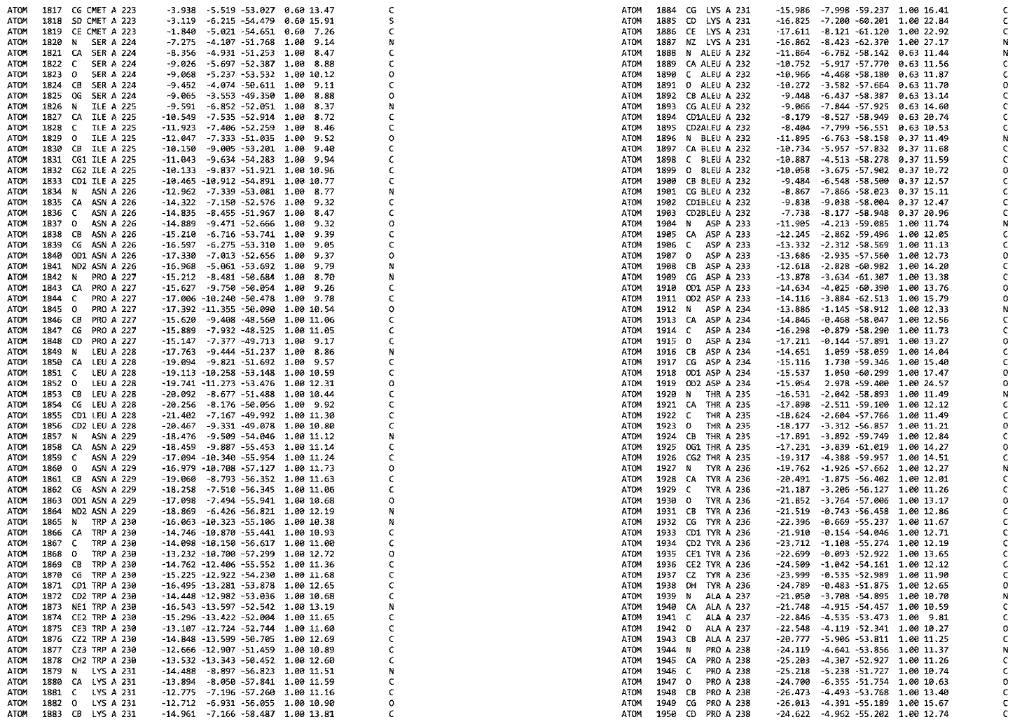

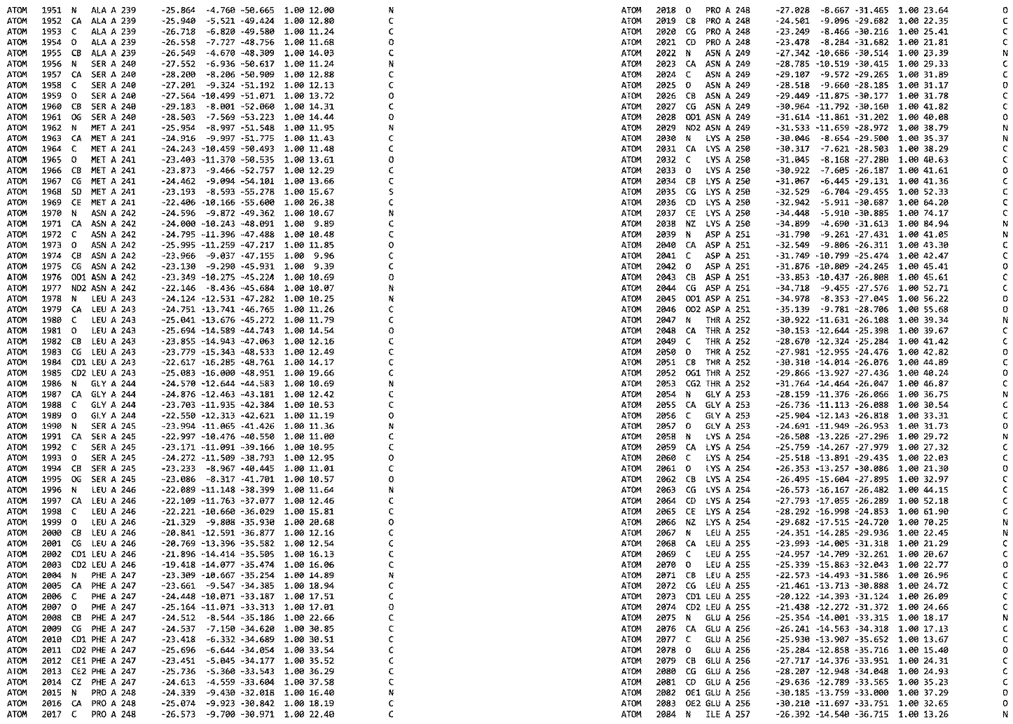

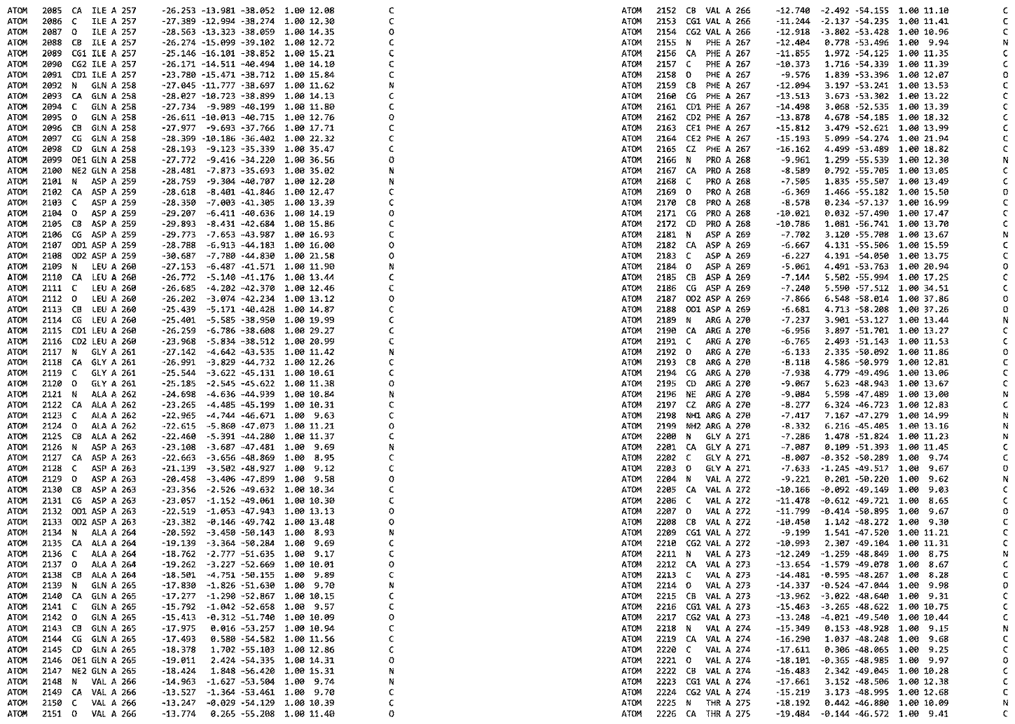

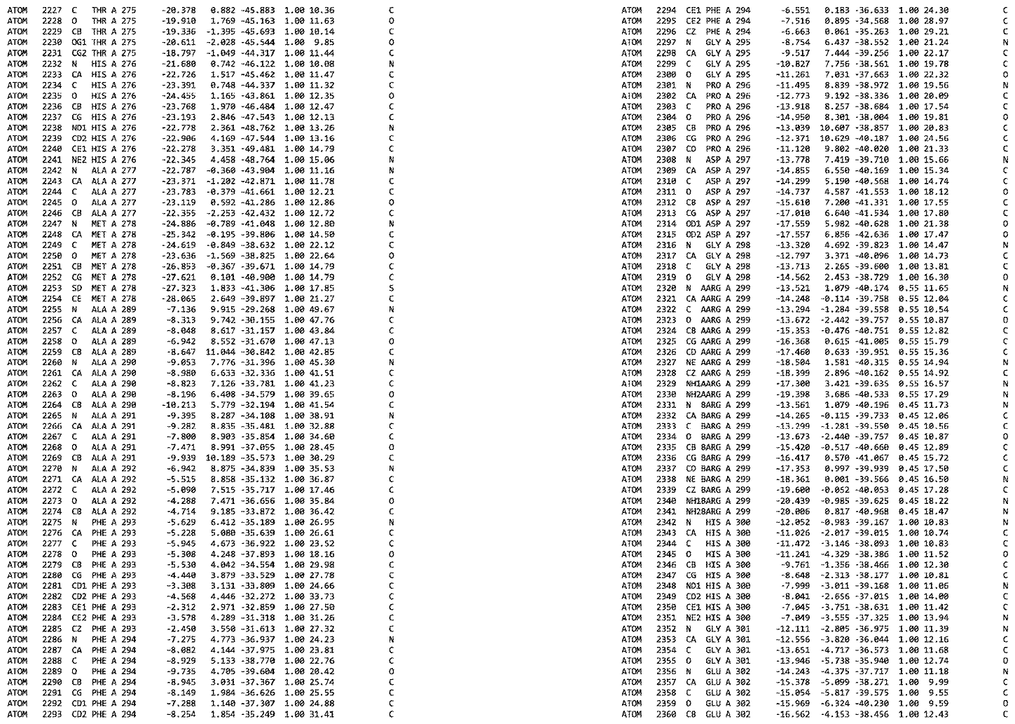

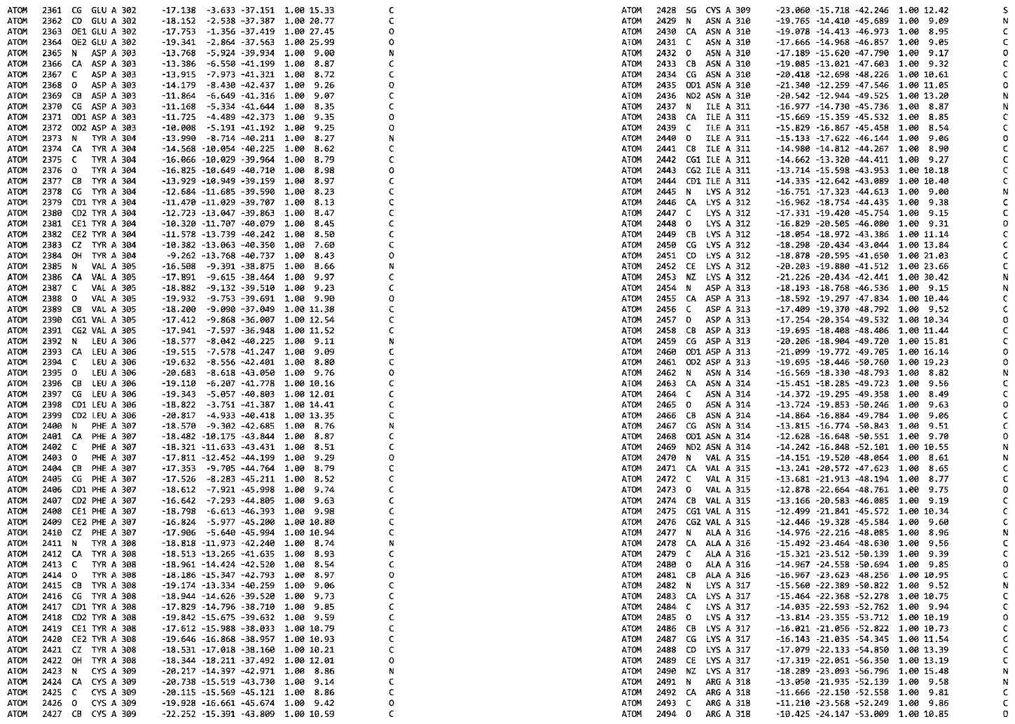

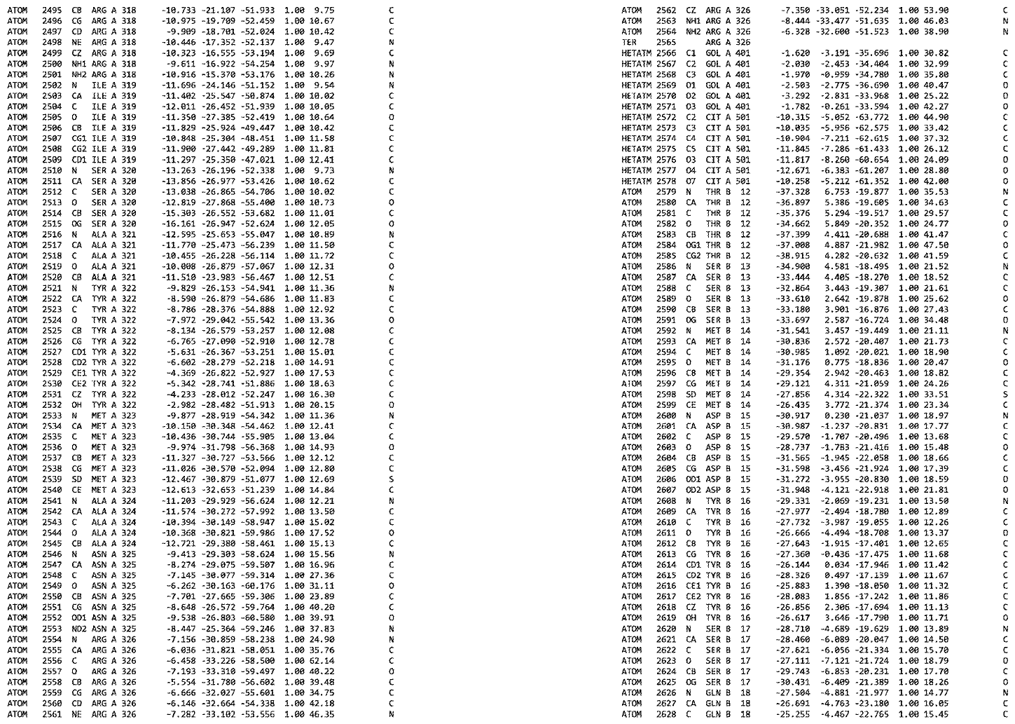

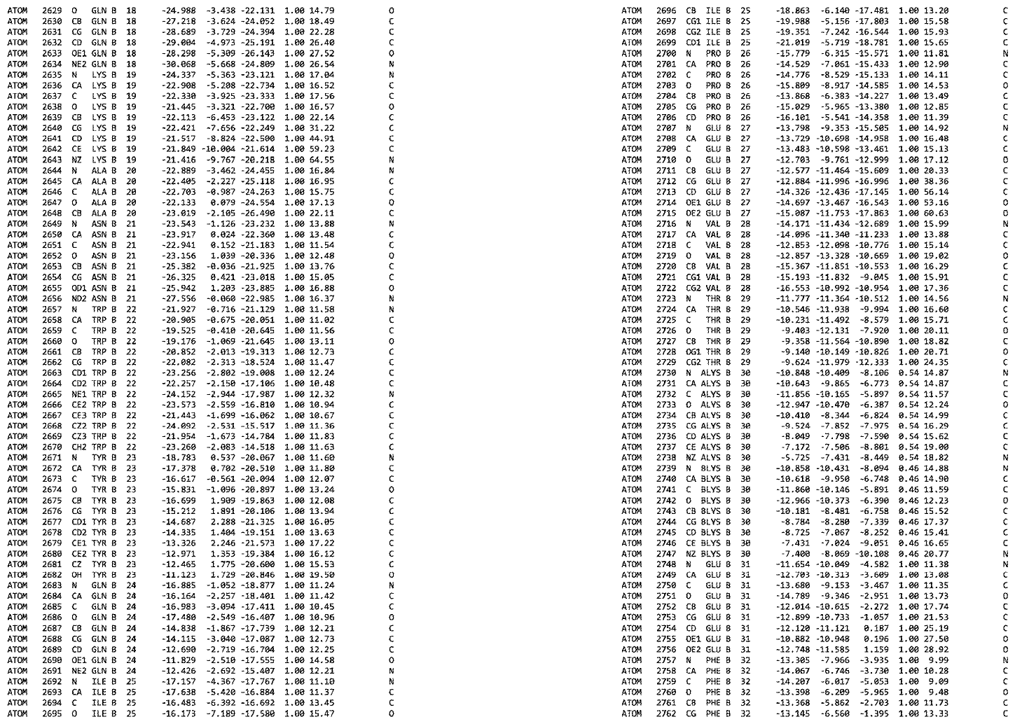

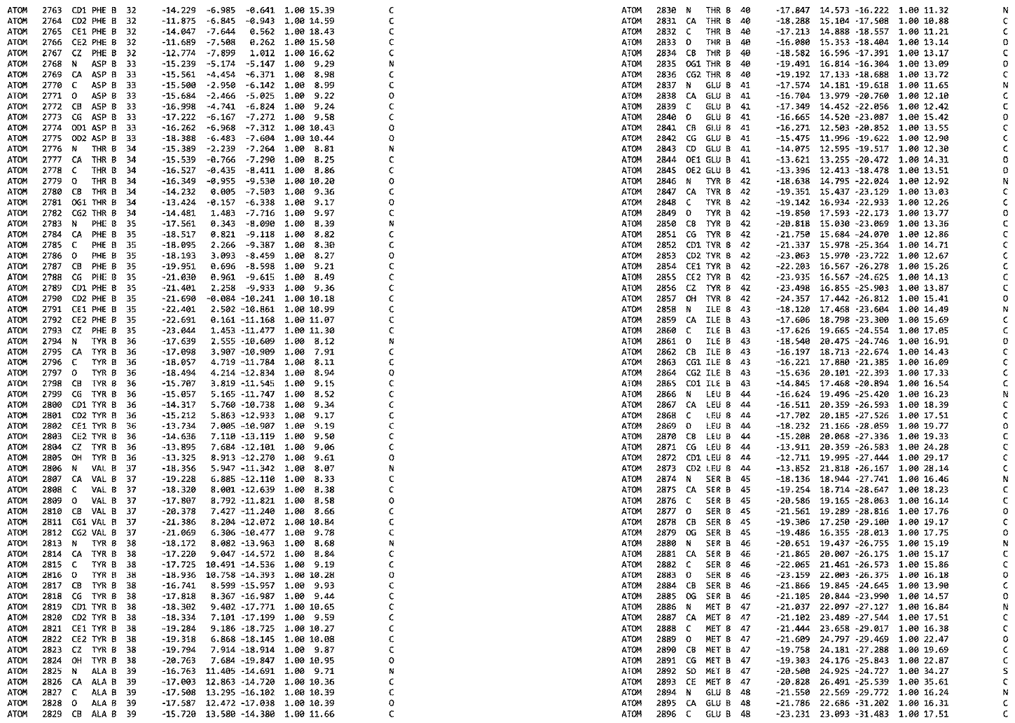

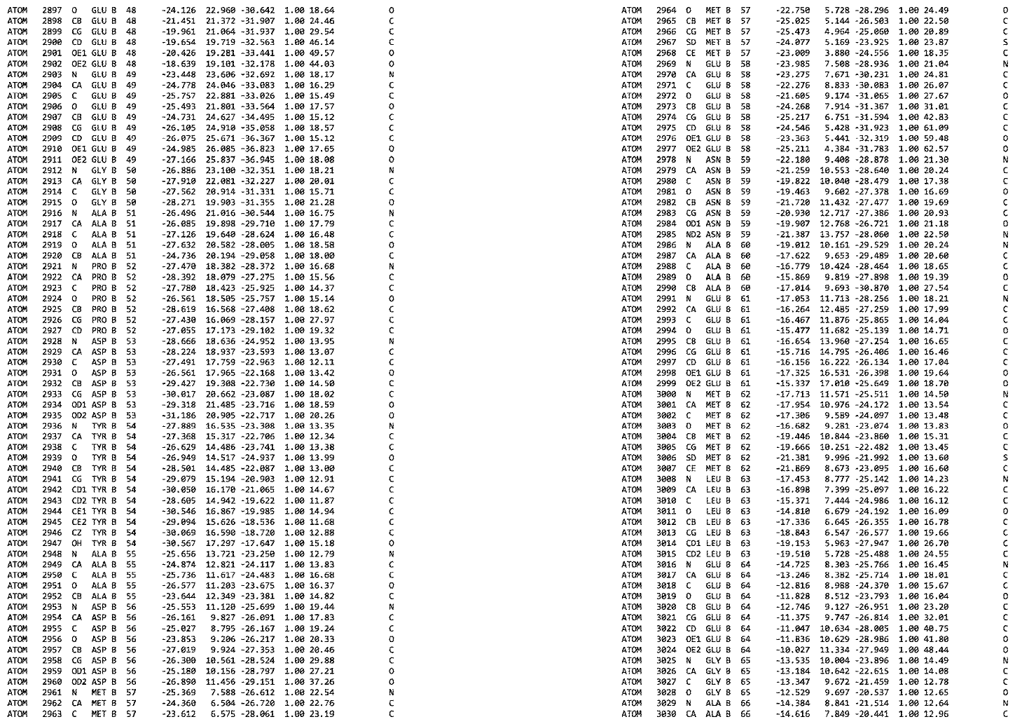

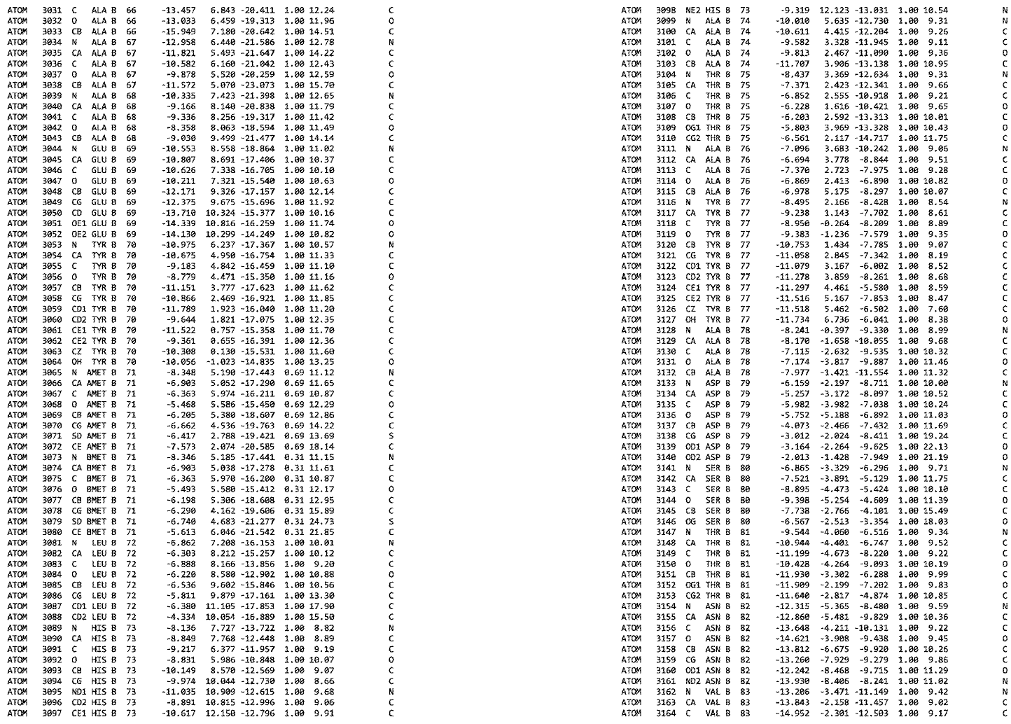

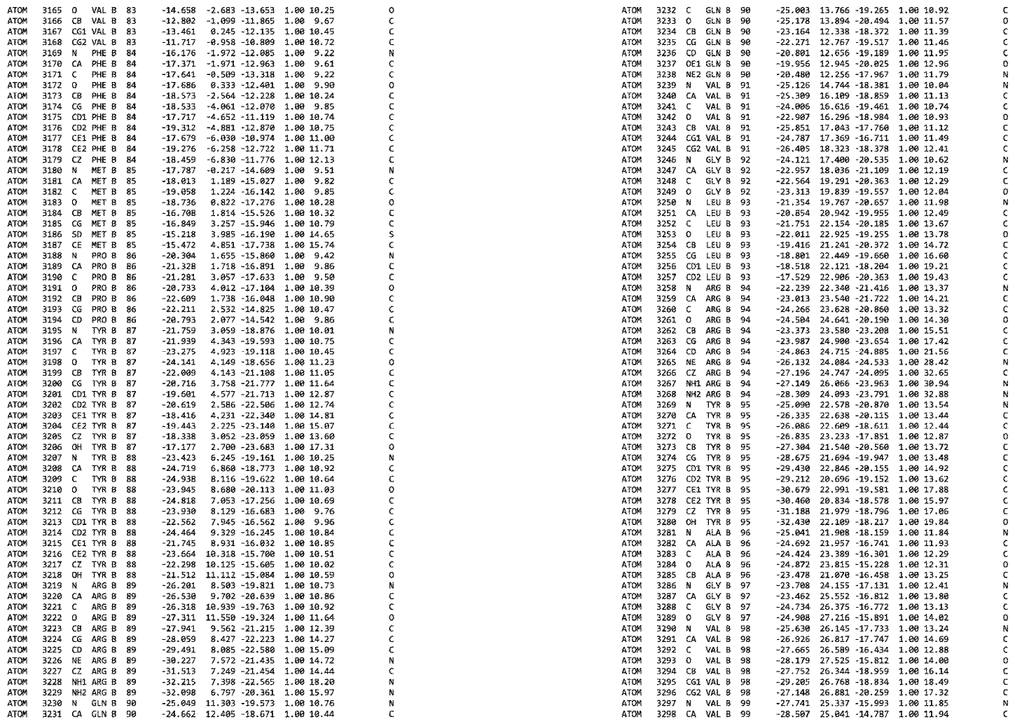

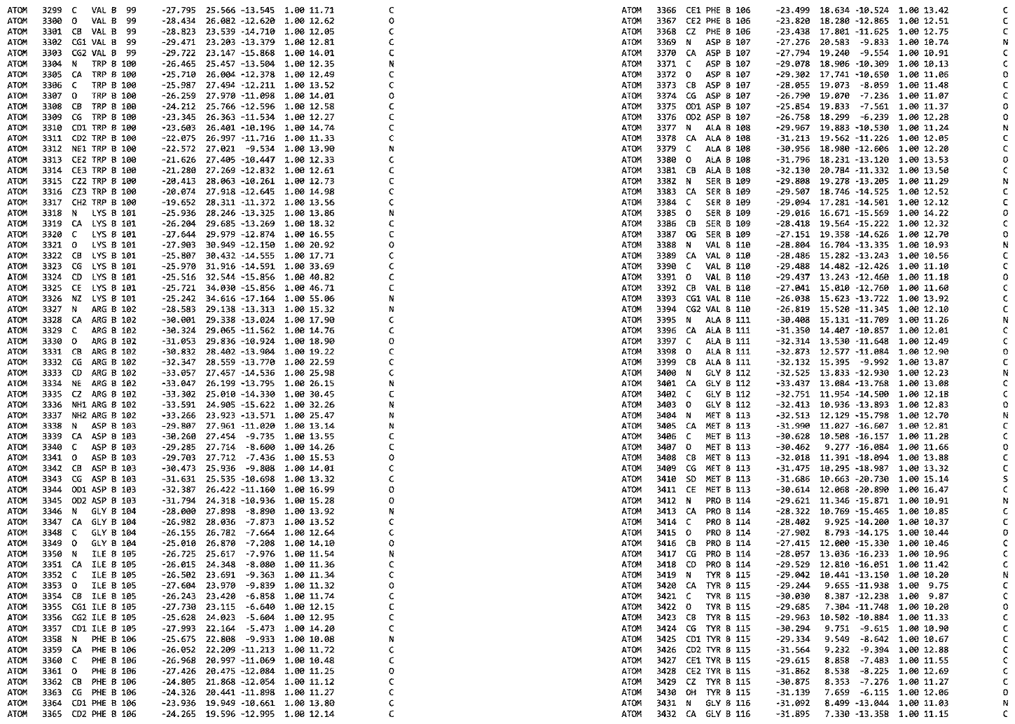

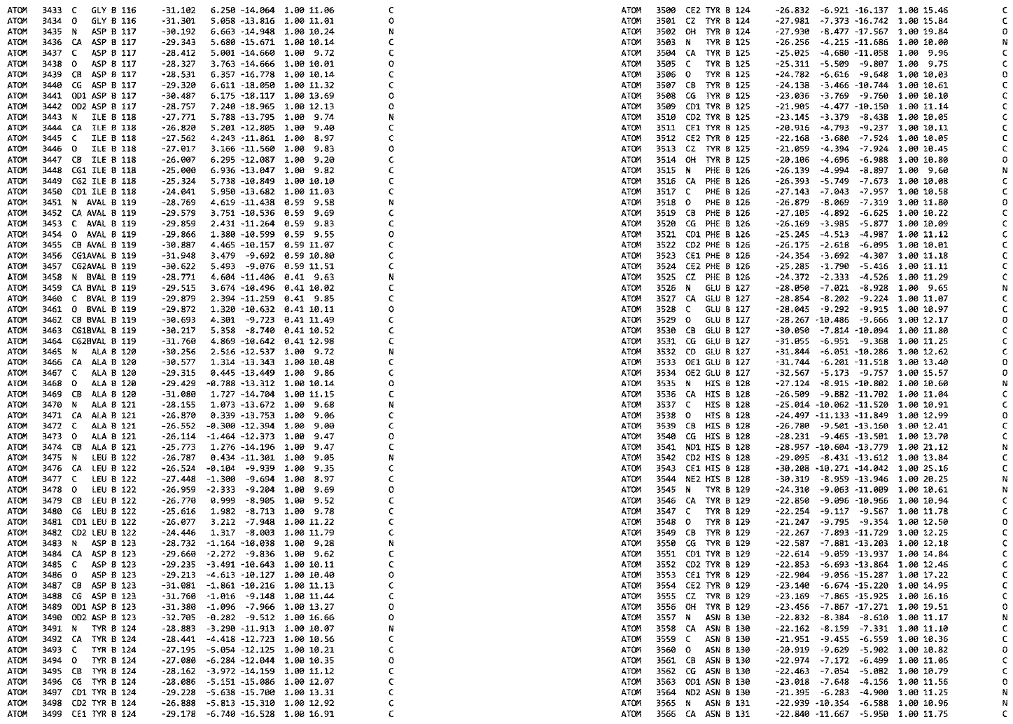

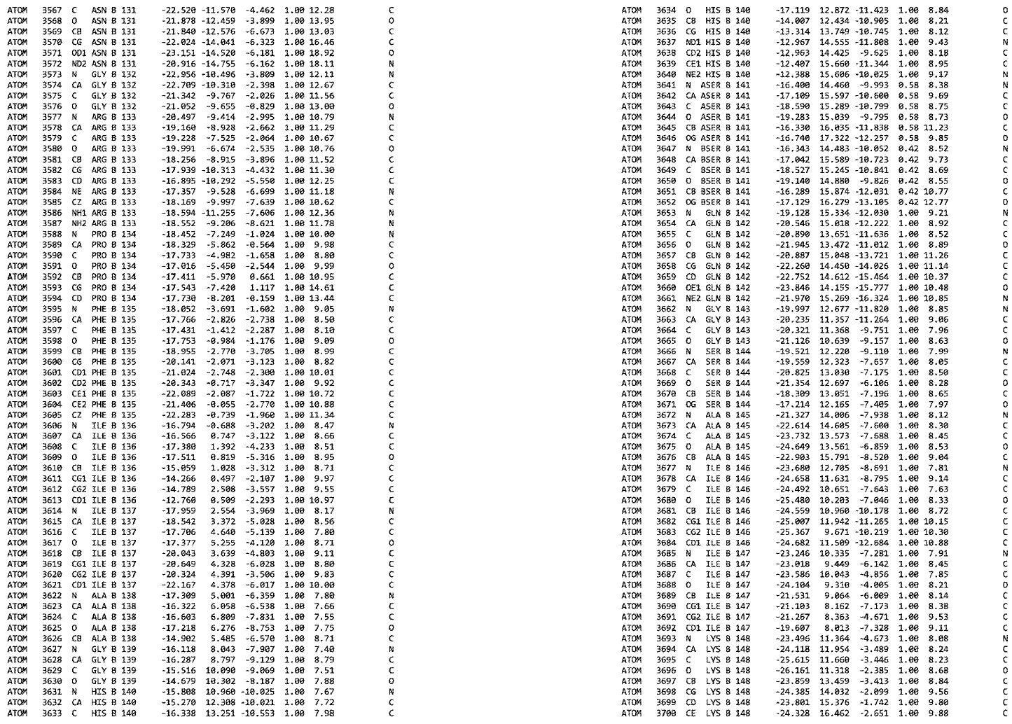

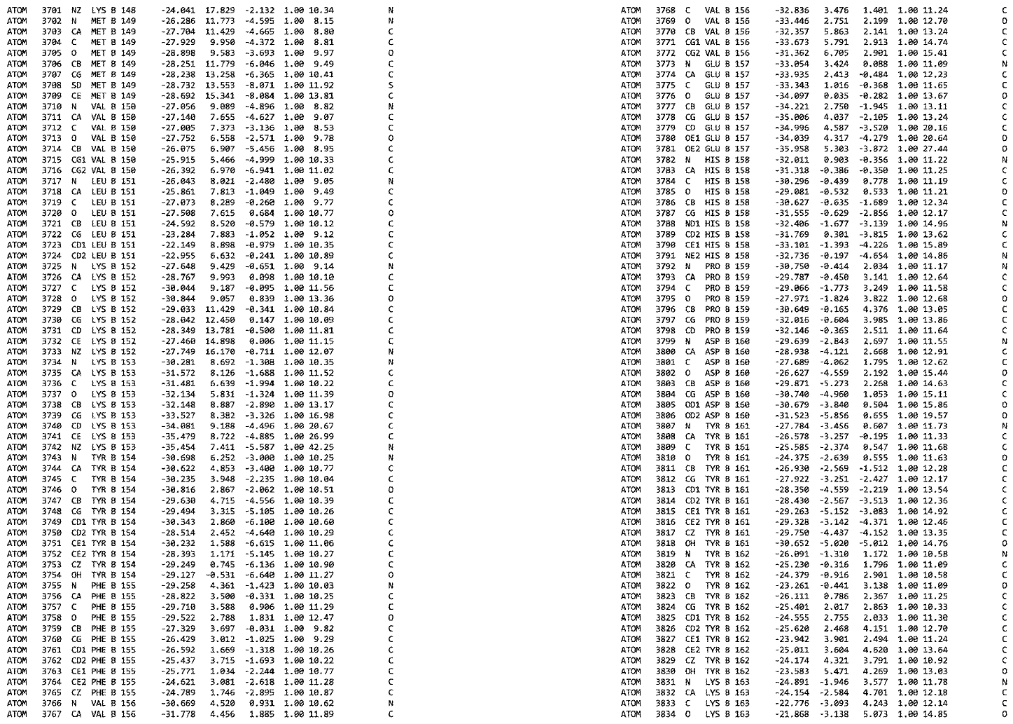

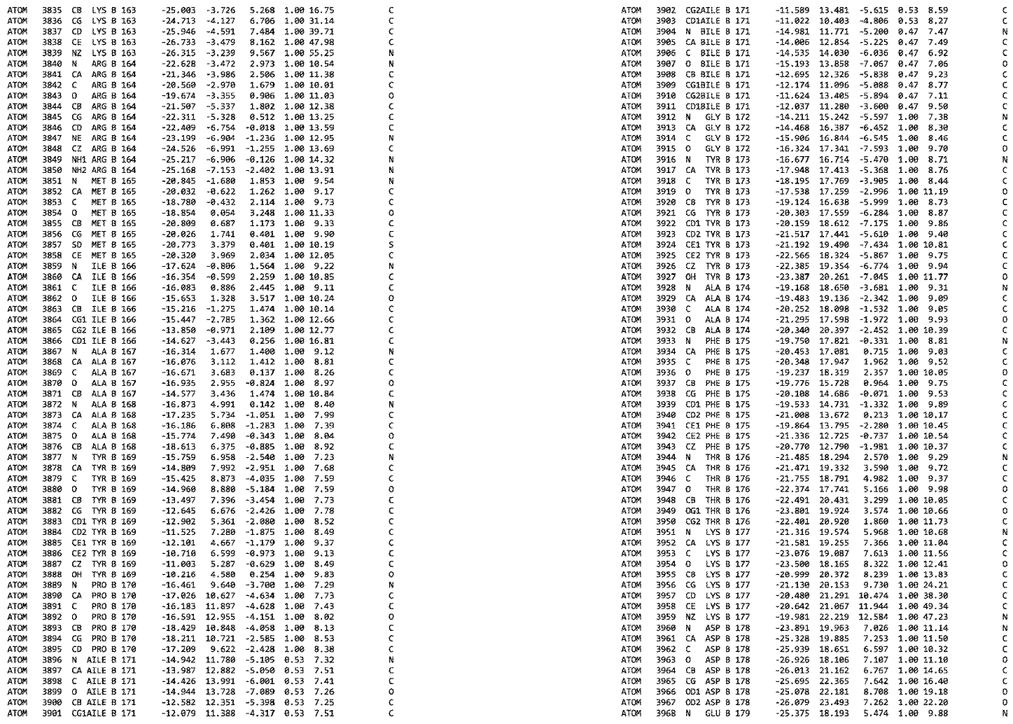

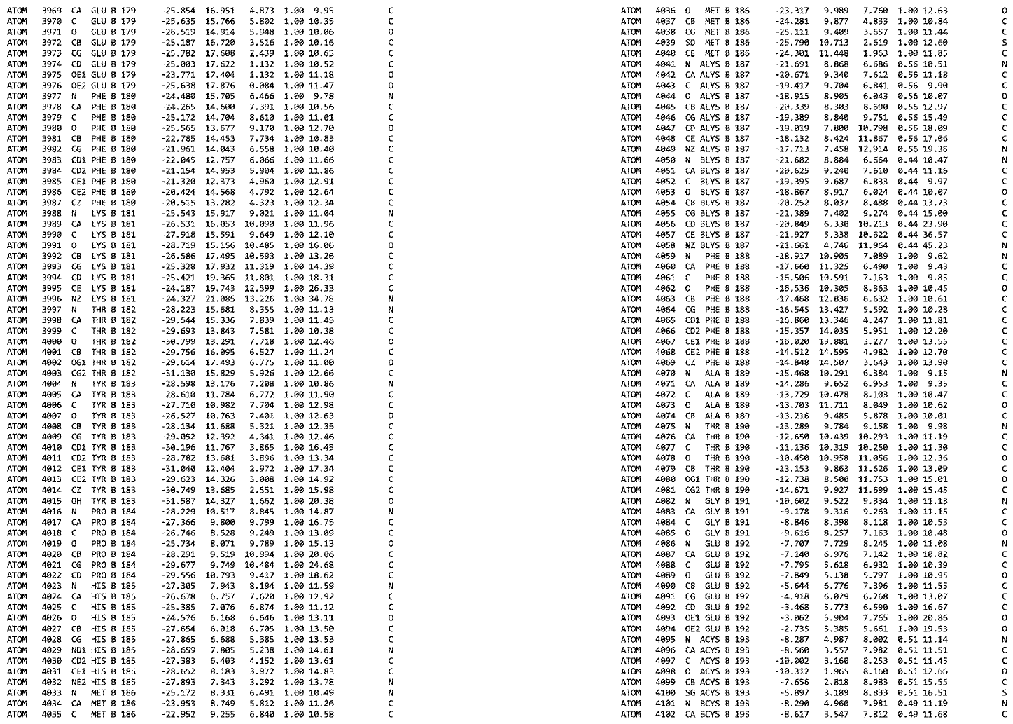

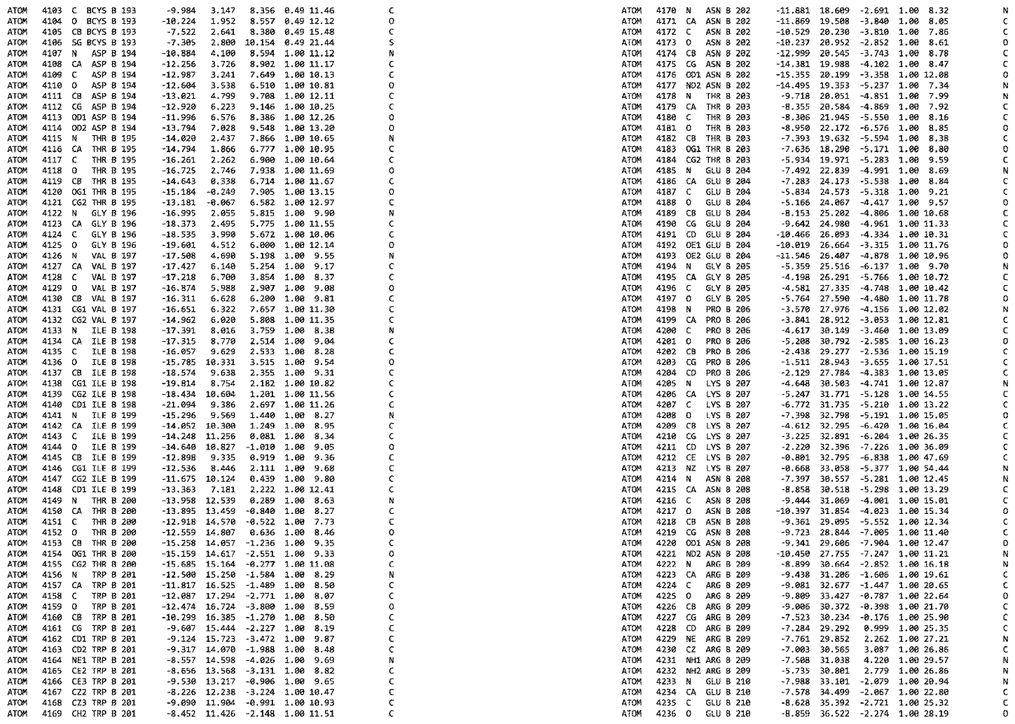

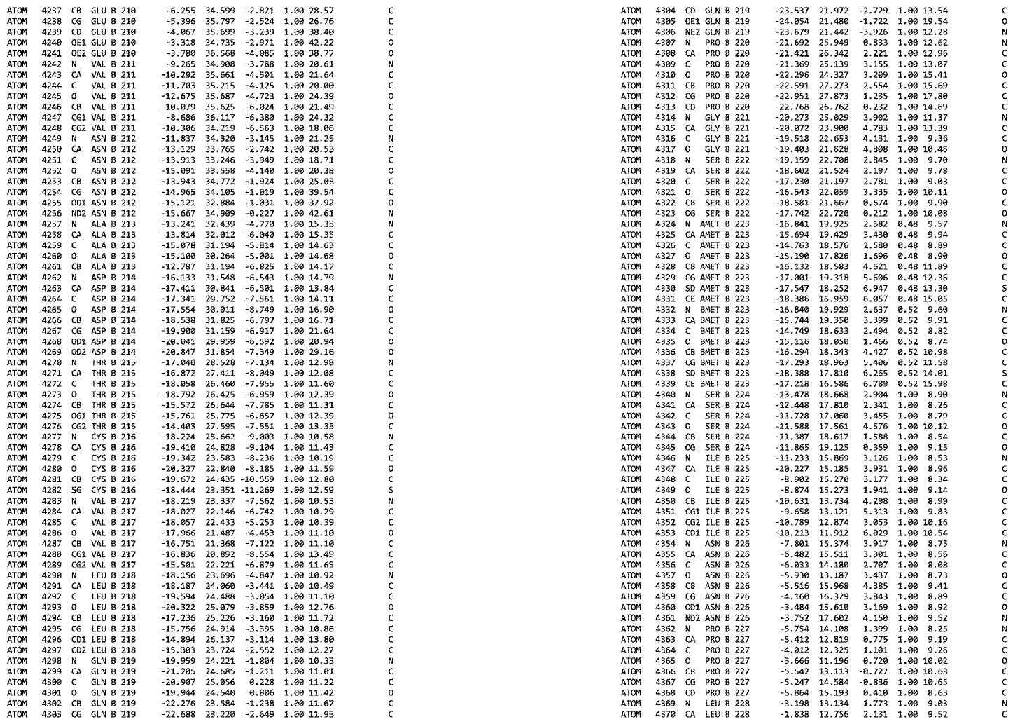

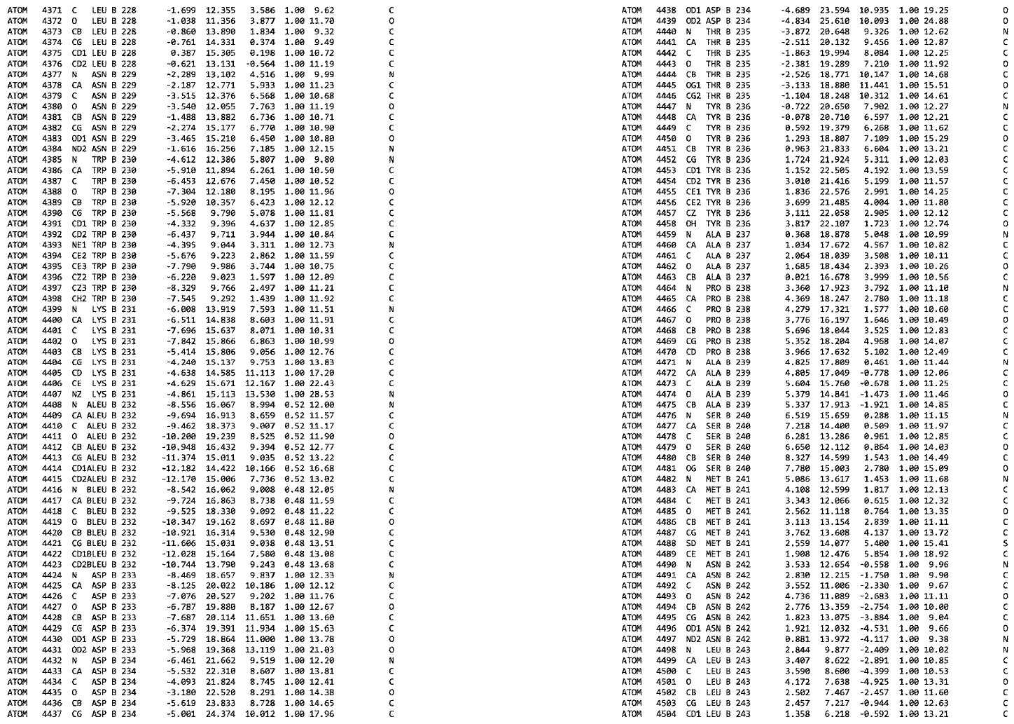

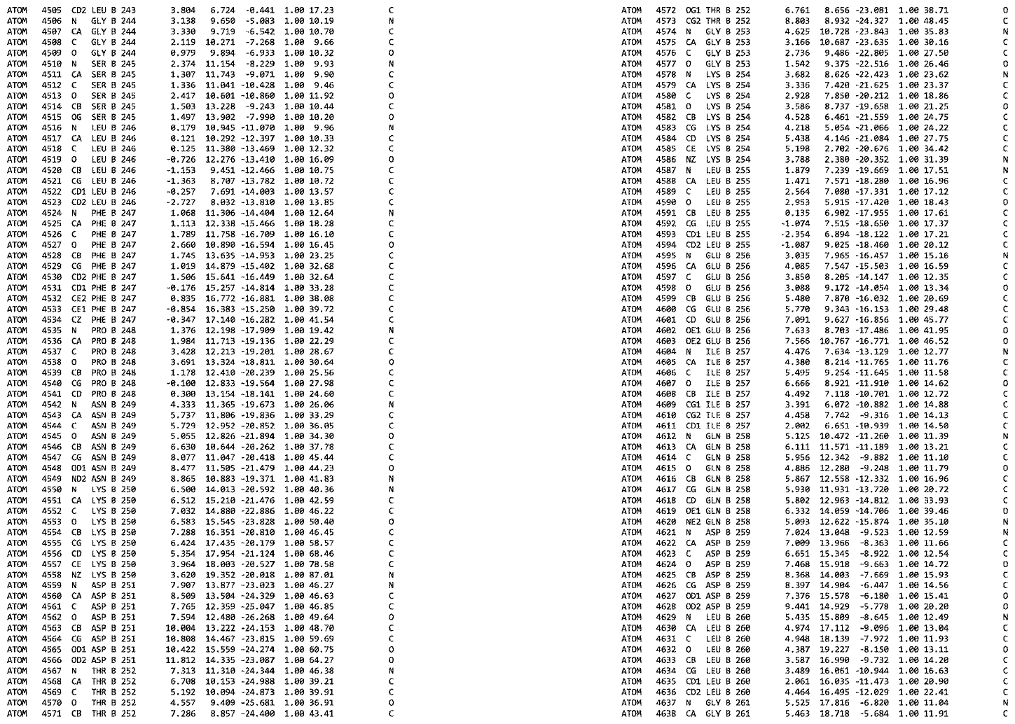

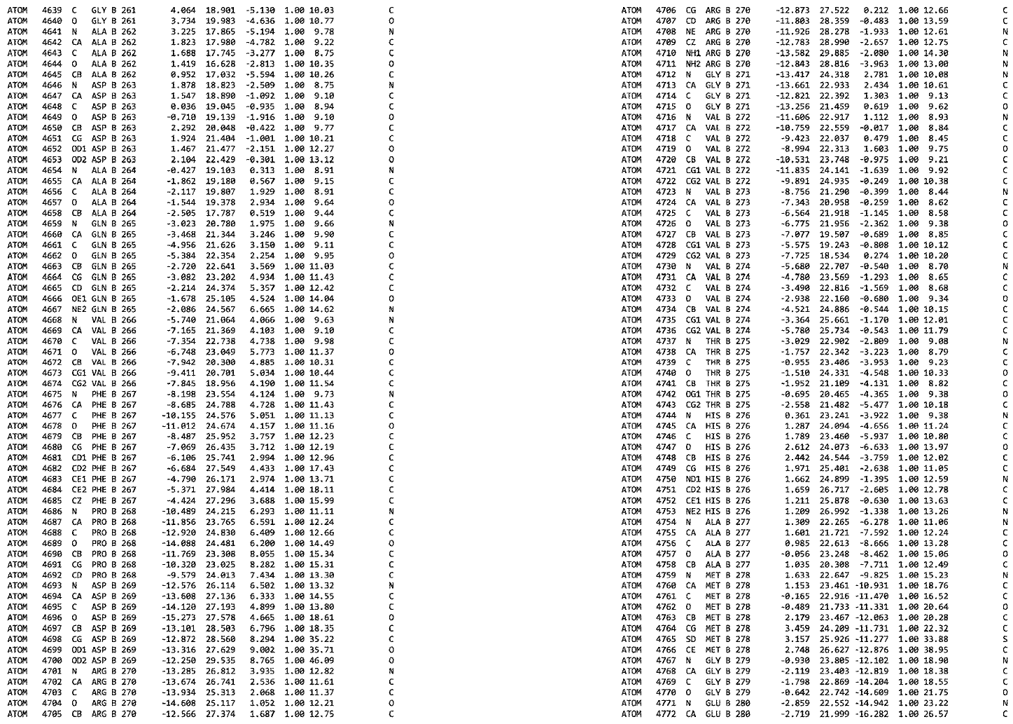

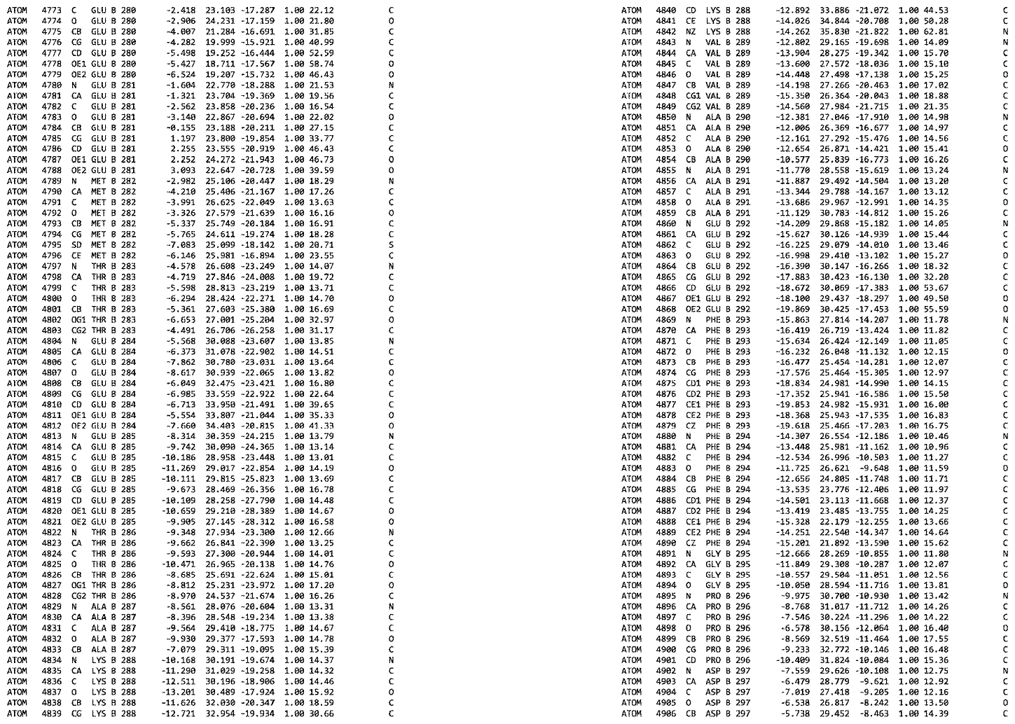

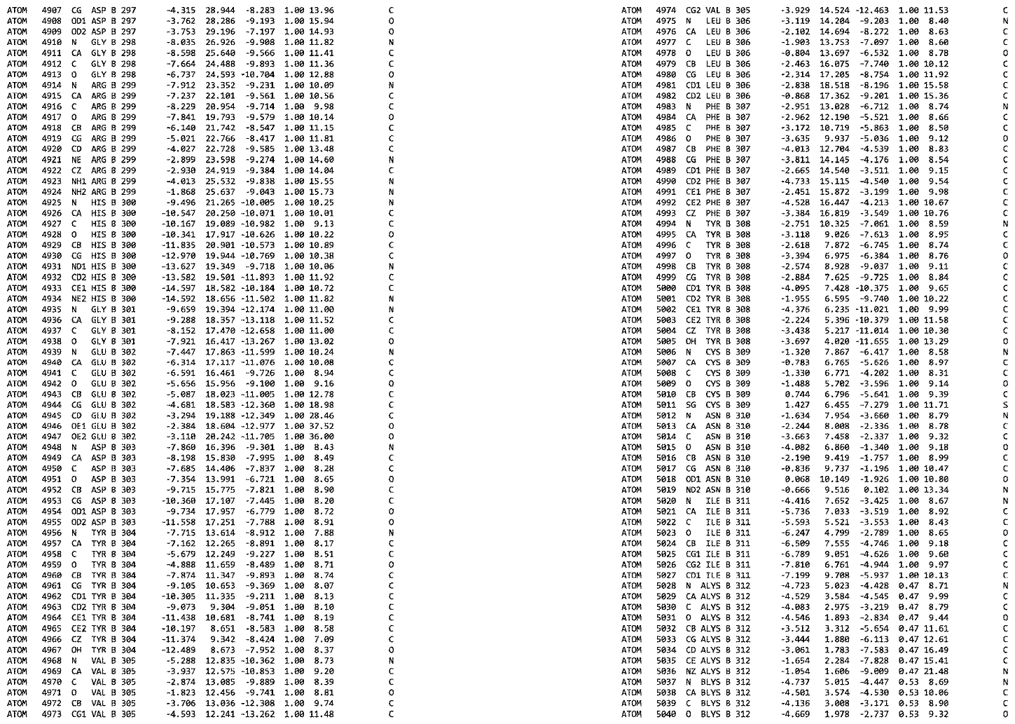

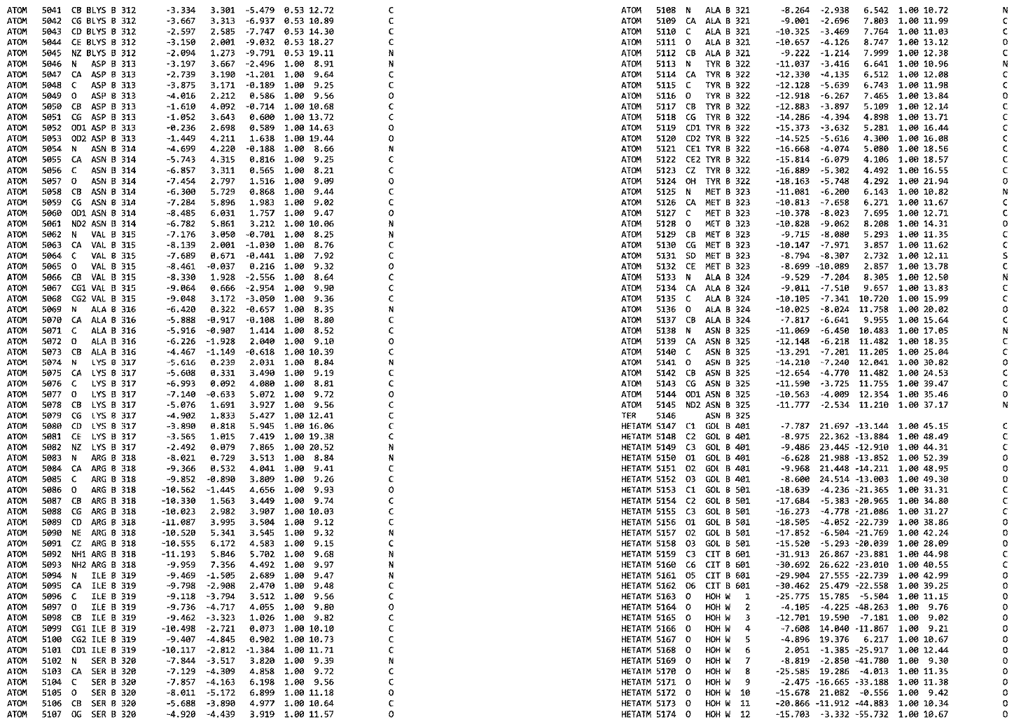

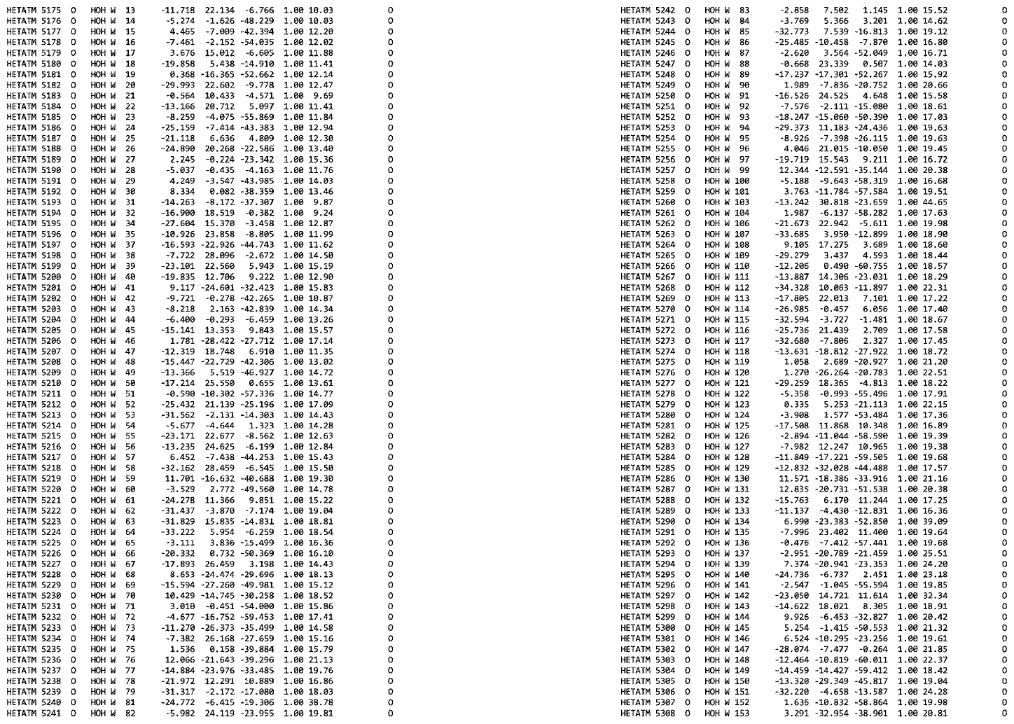

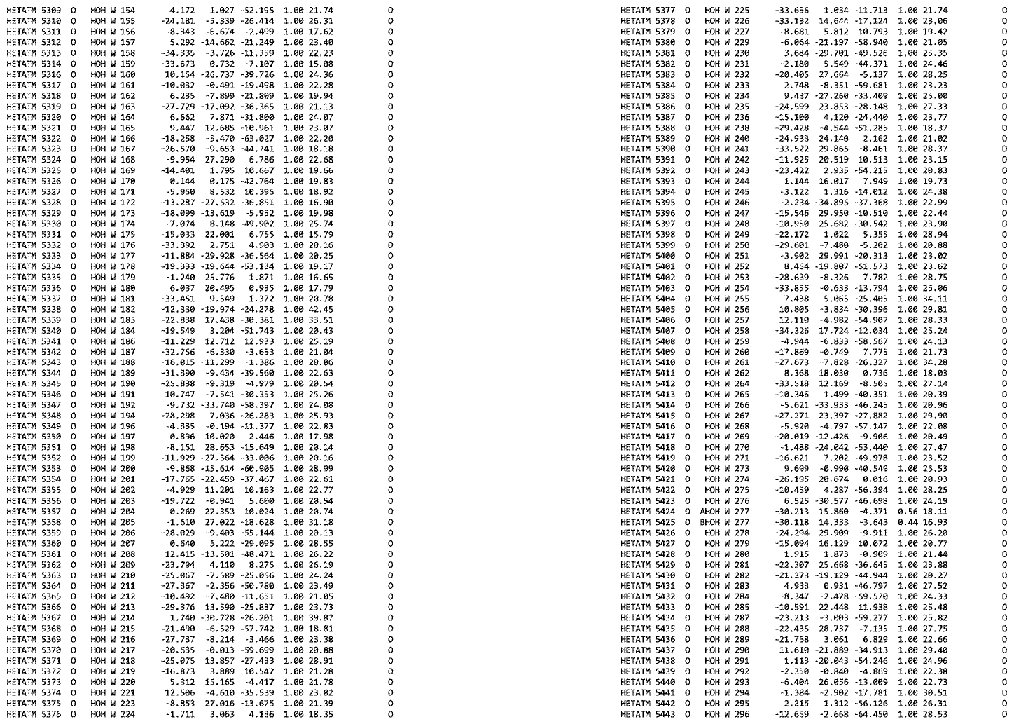

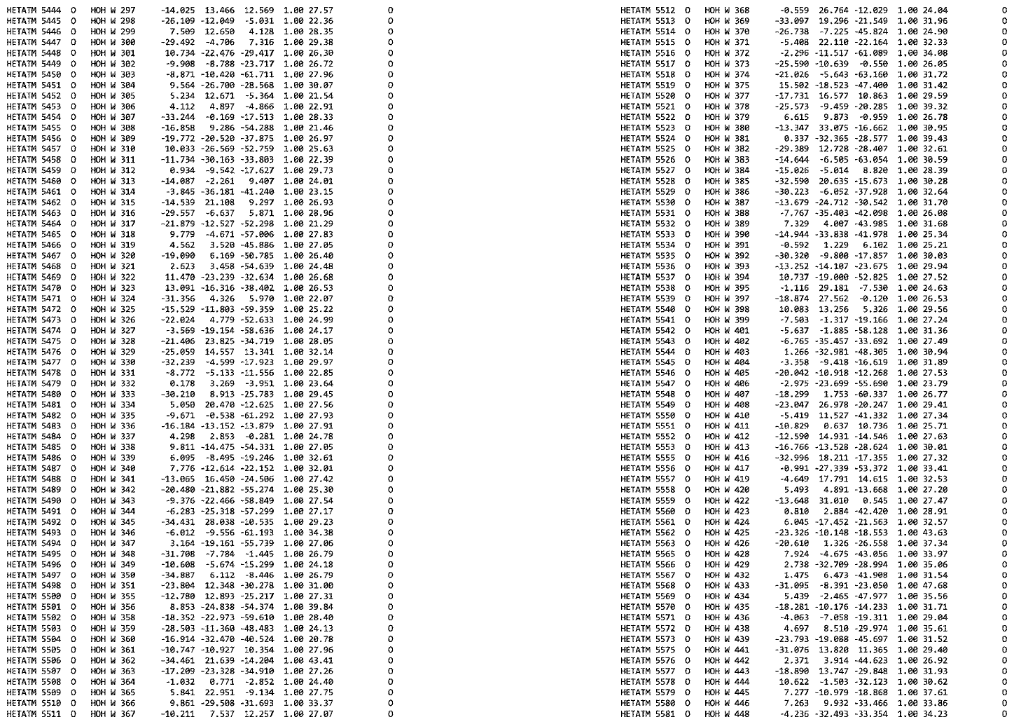

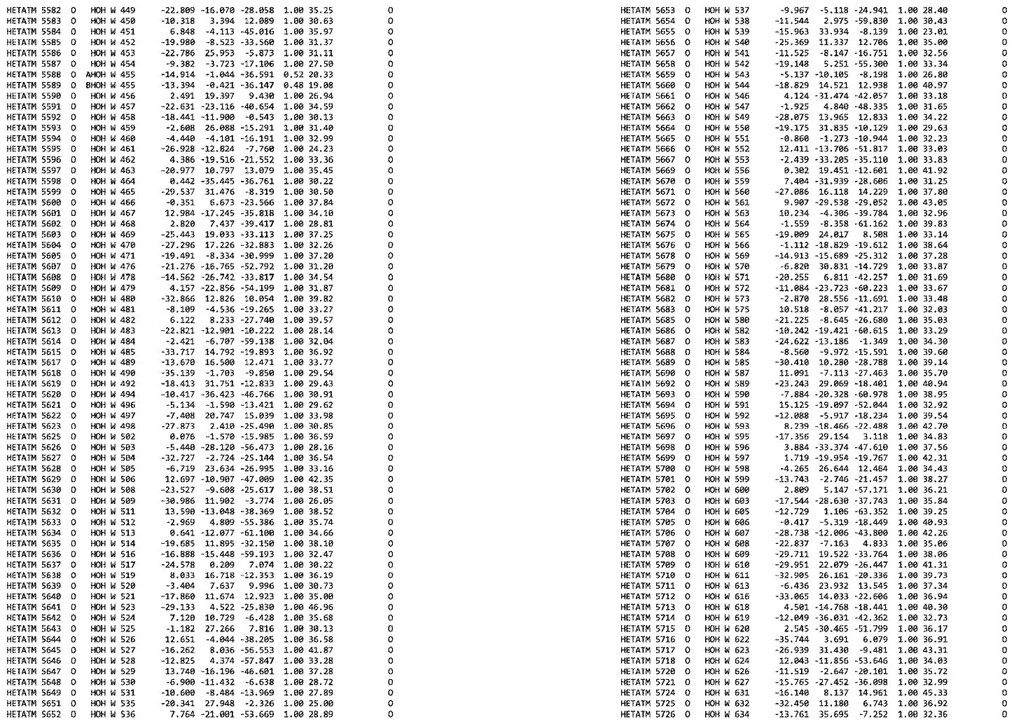

The structure of RR11O2 is shown in FIGS. 7, 8a and 11-14. The atomic co-ordinates are listed in FIG. 16.

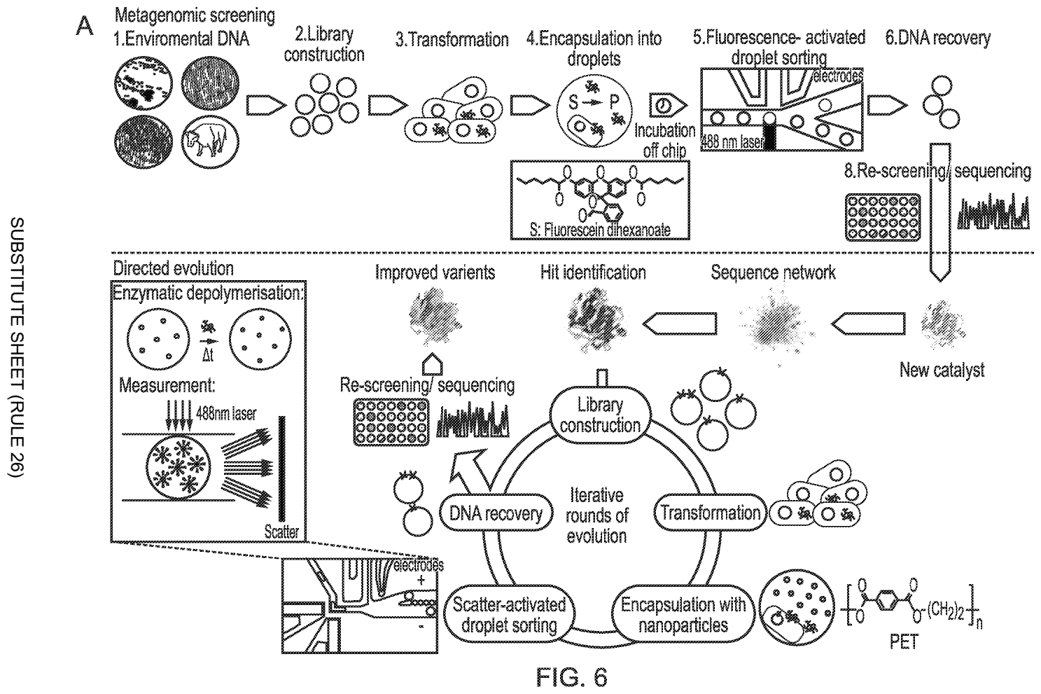

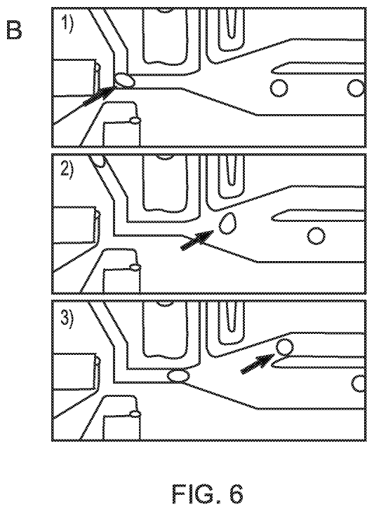

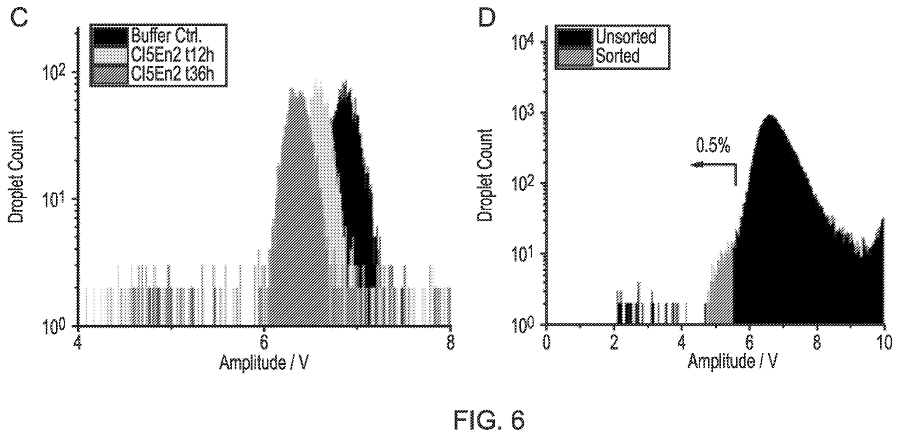

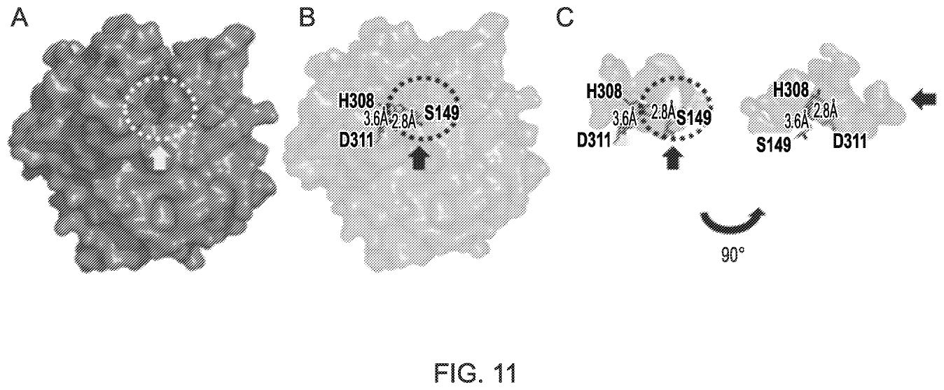

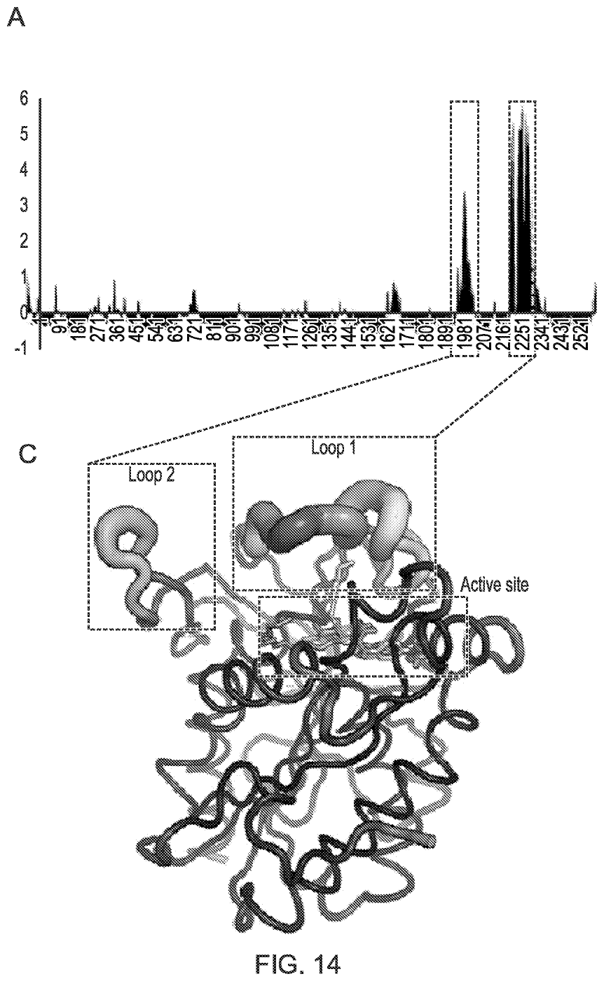

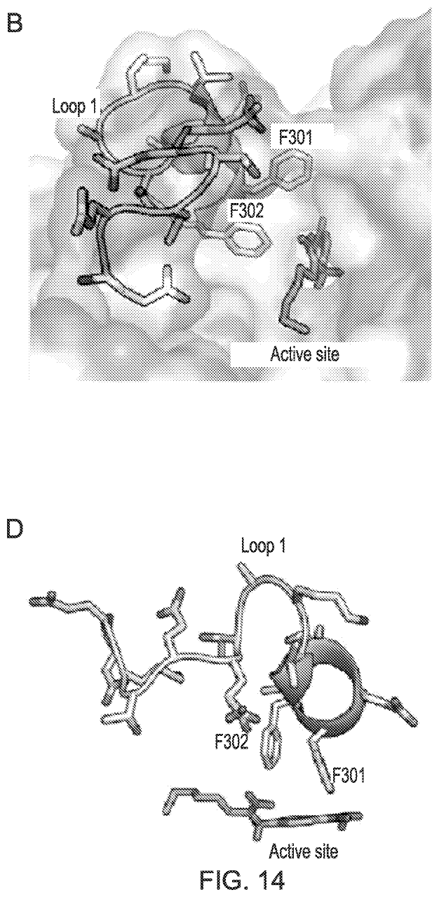

The crystal structure of RR11O2 was determined at the high resolution of 1.1 Å with the space group P 1 21 1, and two molecules in the unit cell. The structure of RR11O2 displays the α/β fold with the catalytic triad composed of S149, D311 and H308 (FIG. 6B) surrounded by an internal cavity with a pocket volume=275.101 volume SA (FIG. 11) close to two flexible loops (FIG. 14), which seems to be associated with the catalytic efficiency displayed by RR11O2 towards the p-NP esters with different lengths. The surface analysis of RR11O2shows a large hollow which provides access for substrates to the active site and catalytic triad which is located at the bottom of the hollow (FIGS. 11A and 14C).

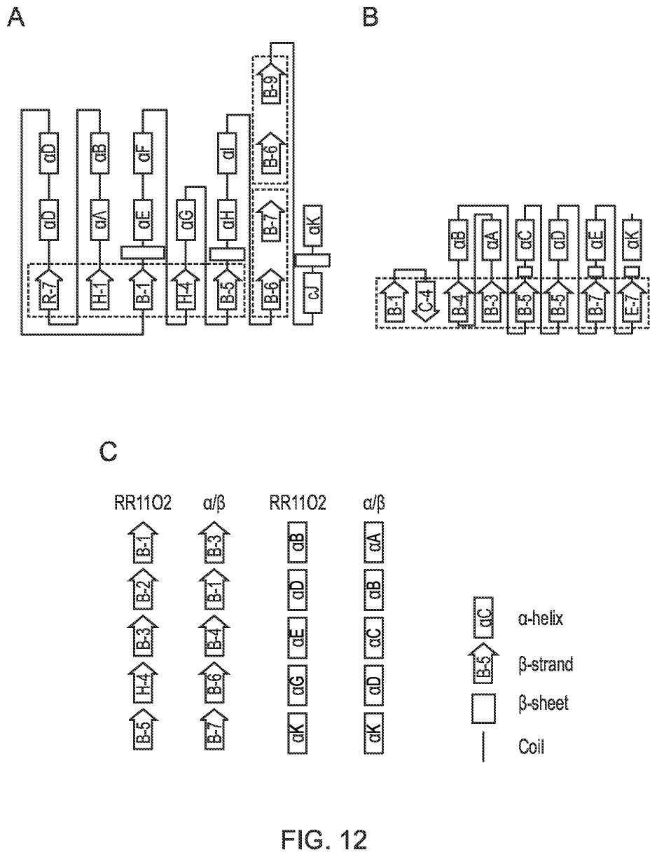

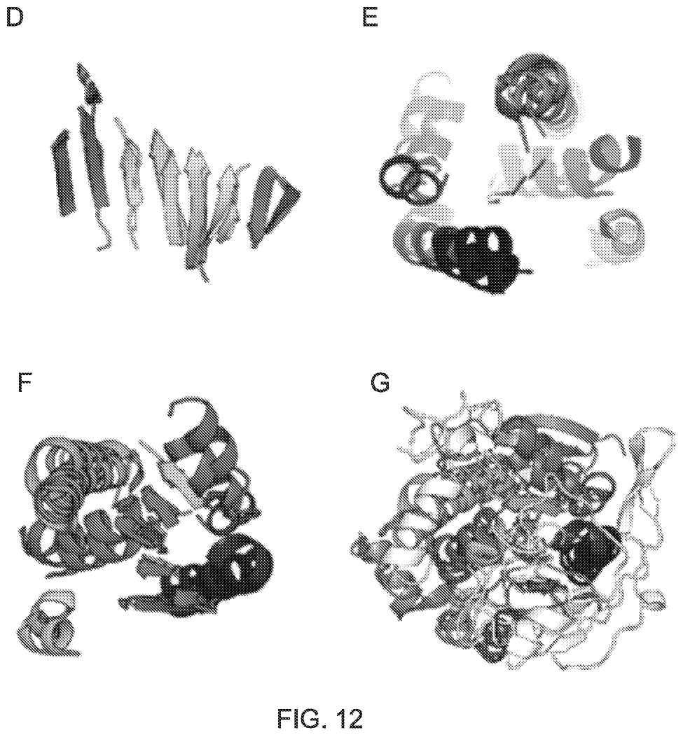

To investigate the degree of structural similarity with others α/β hydrolyses a structural alignment was performed using the Dali server. Although RR11O2 has very low sequence similarity with other members of the α/β family, it was possible to observe the classic α/β fold in RR11O2 and other conserved features with α/β hydrolyses (FIG. 12).

While there are significant differences in the outer loops of RR11O2, the core of RR11O2overlaps with a RMSD of 2.069 Å between the β-sheet of RR11O2 and the β-sheet of a protein (PDB code 4VFO) that displays the traditional α/β hydrolayse fold. In brief, the β-strands of RR11O2 form a smaller β-sheet that aligns with the central β-sheet of the α/β hydrolayse fold (FIG. 12). As with the α/β hydrolayse fold, the central β-sheet of RR11O2 is surrounded by a number of α-helix's but just 5 out of 9 β-strands and 4 out of 9 α-helixes are common to RR11O2 and the other hydrolases.

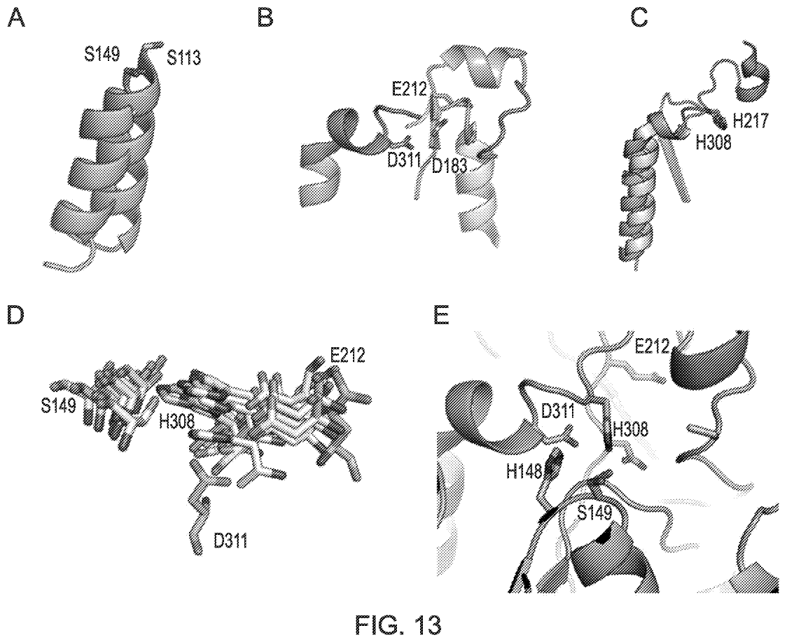

Because of the conservation of key parts of the core structure the positioning of the residues in the catalytic triad are conserved between RR11O2 and the α/β hydrolayse fold (FIG. 13). The catalytic serine (Ser149) and the basic His308 align with the catalytic triad of the hydrolayses identified in the DALI alignment that display the α/β fold. Indeed, the average RMSD between Ser149 and His308 from RR11O2 and the equivalent residues from the top 20 hits of the DALI server is 0.824 Å. Ser149 of RR11O2 is in the conserved motif GXSXG. The third component of the catalytic triad is often an acid (e.g. Asp183 in PDB code 1UFO). However, comparison of RR11O2 with the catalytic triad of structures from the DALI alignment shows that although there is a conserved acidic residue (Glu212) in the correct position, this residue is not correctly orientated. It is likely that this residue can rotate to partake in the catalytic mechanism. However, in order to do this it would have to break its interaction with Arg278.

The identification of the catalytic serine and binding site allows the interesting selectivity profile of RR11O2 for the hydrolysis of esters to be rationalized. The binding pocket consists of 2distinct pockets (A and B) on either side of the catalytic triad. Firstly, there is a covered hydrophobic pocket (pocket A) which is terminated by W108, the conserved hydrophobic clamp. In the structure presented here, the pocket A is occupied by 1 glycerol molecule. This indicates that it is likely that this site will bind small molecules. The second pocket (pocket B) is a deep but open pocket. That is terminated by the flexible loop. The mouth of pocket A could be viewed as too constricted to allow small molecule entry. However, examination of the normalized B-factors from the structure (PDB code 6ZZV) indicate that the loop that covers pocket A is very flexible. Therefore, there is likely to be a significant amount of breathing that would allow pocket A to open so that ligands can enter.

In order to better understand how these substrates bind and how the selectivity of RR11O2 takes place, modelling studies were performed to allow analysis of the binding mode. The linear esters tested in FIG. 8b were docked into the binding site of RR11O2 using GOLD. From these results, binding poses were chosen that were judged to be productive in the reaction mechanism. These productive binding modes were defined as binding modes whereby the carbonyl carbon of the substrates is predicted to be near Ser149 (within 3 Å) and the orientation in relation to the nucleophilic Ser149 is such that it would allow nucleophilic attack on the substrate carbonyl. For the shorter substrates, GOLD can generate a number of productive binding modes where the para-nitrophenol group is either in pocket A or pocket B. However, for longer substrates (e.g. p-NP hexanoate, C6) in order to generate productive binding modes the para-nitrophenol has to bind in pocket A. As the binding mode is likely to be similar for all substrates, it was therefore decided that the correct binding mode is one where para-nitrophenol binds in pocket A (see FIG. 8A). The selectivity data generated shows a high level of hydrolysis for substrates containing from two to eight carbons in the alkoxy chain. Where upon, the activity drops of significantly for para-nitrophenol esters with longer chain than eight carbons. Analysis of the predicted binding modes indicates that esters with alkoxy chain length longer than eight carbons reach outside the pocket identified by PRANK.

### Example 9: Structural Characterization of C15En2-A9S

### Crystallisation of CI5En2

To the purified recombinant protein pExp-bla CI5En2-A9S (9.3 mg of protein) was added 600 units of TEV protease (Sigma-Aldrich, Cat. N° T4455-1KU) to cleave the β-lactamase fusion tag. The cleavage was performed at 4° C. for 16 h and was confirmed via SDS-Page gel, being the fusion tag removed via nickel purification.

After purification, the cleaved and pure CI5En2-A9S protein at final concentration of 6.7 mg/mL in buffer containing 100 mM hepes pH 7.0, 100 mM sodium formate was submitted to initial crystallization trials by using the following commercial kits: JCSG suite (Qiagen, Hilden, Germany), Wizard I and II (Emerald BioSystems, Bainbridge Island, Washington, USA) and Classics (Jena Bioscience, Germany). The first crystals were obtained in 0.8 M Na2Succinate (commercial kit JCSG, well F7), and they were further optimised by changing salt and protein concentration.

We also co-crystallised CI5En2-A9S in the presence of PCL nanoparticles. For that, plates were manually prepared through vapour-diffusion technique using 1 μL: 1 μL and 1 μL: 0.5 μl rate of protein and mother liquor. All crystallisation plates were incubated at 18° C.

### Data Collection and Structure Determination of Apo-CI5En2-A9S

The crystals were submitted to diffraction at the beamline 103 from Diamond Light Source, Oxfordshire, United Kingdom, and images were recorded using a Pilatus 6M detector.

Datasets of apo form CI5En2-A9S and co-crystallised with PCL were indexed and integrated using XDS (KABSCH, 2010). All structures were solved by molecular replacement using Phaser program in CCP4 package. The structure coordinates of Thermobifida fusca cutinase (PDB code 5ZOA), which shares 36.39% of amino acid sequence identity with CI5En2, was used as a model. Refinement of the structures was manually performed using COOT and then subjected to rigid body and restrained refinement cycles using the program REFMAC5. Protein validation parameters were evaluated according to Ramachandran plot. Analysis of the structures and figures were made with Pymol. All statistics from data collection and refinement are shown in the table below:

| Data collection Date | Nov. 26, 2018 | |
|---|---|---|
| Beamline | DIAMOND BEAMLINE I03 | |
| Deposition Session | D_1292113738 | |
| pdb | 7NCQ | |
| Datacollection | | |
| Wavelength | 0.9762 | |
| Resolution range | 106.182-2.476 | (2.519-2.476) |
| Space group | P 62 2 2 | |
| | 122.608 122.608 218.522 90 90 | |
| Unit cell | 120 | |
| Total reflections | 1333237 | (69226) |
| Unique reflections | 35059 | (35059) |
| Multiplicity | 38.0 | (40.5) |
| Completeness (%) | 99.3 | (100.0) |
| Mean I/sigma(I) | 3.8 | (1.0) |
| Wilson B-factor | | |
| R-merge | 3.241 | (65.753) |
| R-meas | 3.284 | (66.600) |
| R-pim | 0.526 | (10.518) |
| CC½ | 0.967 | (0.663) |
| Refinement | | |
| Resolution range | 106.18-2.48 | (2.48-2.540) |
| Reflections used in refinement | 33250 | (2359) |
| Reflections used for R-free | 1742 | (136) |
| R-work | 0.21235 | (0.402) |
| R-free | 0.24235 | (0.484) |

| | |
|---|---|
| CC(work) | 0.952 |
| CC(free) | 0.944 |
| Number of non-hydrogen atoms | 4105 |
| macromolecules | 3919 |
| ligands | 122 |
| solvent | 64 |
| Protein residues | 532 |
| RMS(bonds) | 0.015 |
| RMS(angles) | 1.93 |
| Ramachandran favored (%) | 94.51 |
| Ramachandran allowed (%) | 5.11 |
| Ramachandran outliers (%) | 0.38 |
| Rotamer outliers (%) | 4.05 |
| Clashscore | 4.84 |
| Average B-factor | 60.44 |
| macromolecules | 59.95 |
| ligands | 82 |
| solvent | 49.56 |

Data for the complex structures of CI5En2 with PCL and BHET is provided in the table below:

| Protein | PCL CI5En2-A9S | | BHET CI5En2-A9S | |
|---|---|---|---|---|
| Data collection Date | 15 Jul. 2021 | | 1 May 2021 | |
| Beamline | DIAMOND BEAMLINE I04 | | DIAMOND BEAMLINE I04 | |
| pdb | 7ZJA | | 7ZJ9 | |
| Datacollection | | | | |
| Wavelength | 0.9795 | | 0.97934 | |
| Resolution range | 76.1-1.90 | (1.90-1.93) | 76.55-2.08 | (2.12-2.08) |
| Space group | P 62 22 | | P 62 2 2 | |
| Unit cell | 122.37 122.37 218.64 90.00 | | 124.01 124.01 218.30 90.00 | |
| | 90.00 120.00 | | 90.00 120.00 | |
| Total reflections | 2904275 | (141563) | 2424227 | (123133) |
| Unique reflections | 76626 | (2741) | 60081 | (2939) |
| Multiplicity | 37.9 | (37.8) | 40.3 | (41.9) |
| Completeness (%) | 100.0 | (100.0) | 100.0 | (100.0) |
| Mean I/sigma(I) | 5.2 | (0.2) | 7.6 | (0.3) |
| Wilson B-factor | 27.9 | | 49.62 | |
| R-merge | 0.31 | (6.932) | 0.813 | (3.76) |
| R-meas | 0.318 | (7.115) | 0.825 | (3.81) |
| R-pim | 0.07 | (1.599) | 0.179 | (8.18) |
| CC½ | 0.998 | (0.478) | 0.999 | (0.415) |
| Refinement | | | | |
| Resolution range | 76.09-1.90 | (1.95-1.90) | 76.55-2.08 | (2.135-2.081) |
| Reflections used in | 64289 | (1842) | 56653 | (3773) |
| refinement | | | | |
| Reflections used for R- | 3373 | (81) | 2953 | (208) |
| free | | | | |
| R-work | 0.253 | (0.675) | 0.2221 | (0.436) |
| R-free | 0.679 | (0.675) | 0.27096 | (0.399) |

| | | |
|---|---|---|
| CC(work) | | |
| CC(free) | | |
| Number of non-hydrogen | 4161 | 4101 |
| atoms | | |
| macromolecules | 3934 | 3914 |
| ligands | 104 | 124 |
| solvent | 123 | 63 |
| Protein residues | 532 | 531 |
| RMS(bonds) | 0.013 | 0.014 |
| RMS(angles) | 1.65 | 1.79 |
| Ramachandran favored | 96.21 | 95.83 |
| (%) | | |
| Ramachandran allowed | 3.79 | 3.98 |
| (%) | | |
| Ramachandran outliers | 0 | 0.19 |
| (%) | | |
| Rotamer outliers (%) | 4.27 | 6.57 |
| Clashscore | 8.81 | 7.59 |
| Average B-factor | 54.91 | 68.34 |
| macromolecules | 54.69 | 67.34 |
| ligands | 66.92 | 106.44 |
| solvent | 51.62 | 55.64 |

### PDB Deposit

The structures of Apo-CI5En2-A9S, PCL-CI5En2-A9S and BHET-CI5En2-A9S have been deposited on PDB and can be accessed by using the PDB codes 7NCQ, 7ZJA and 7ZJ9 respectively.

### Results

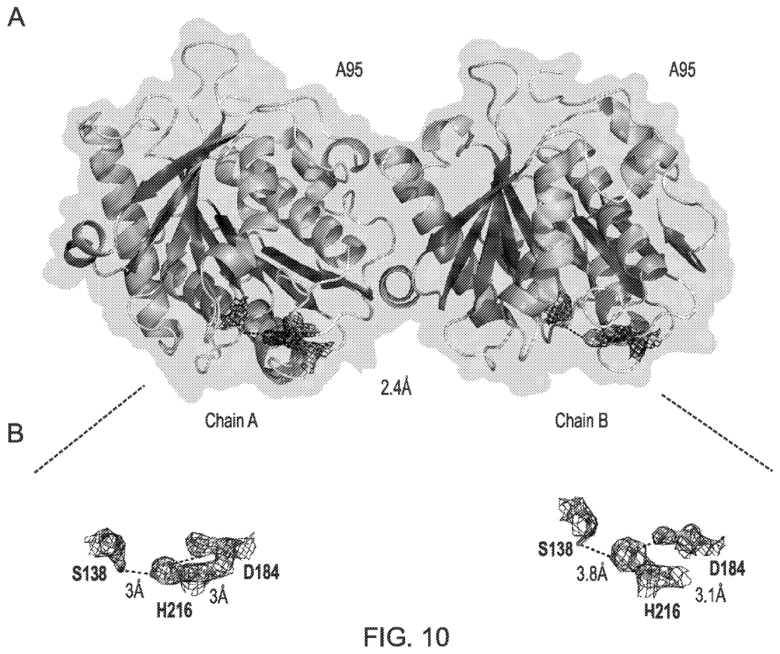

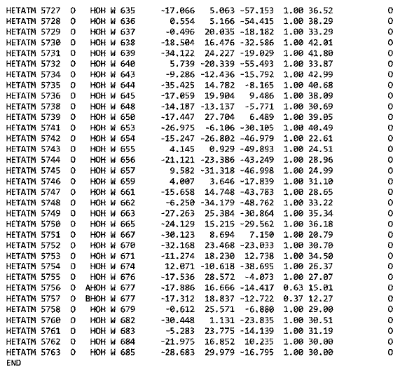

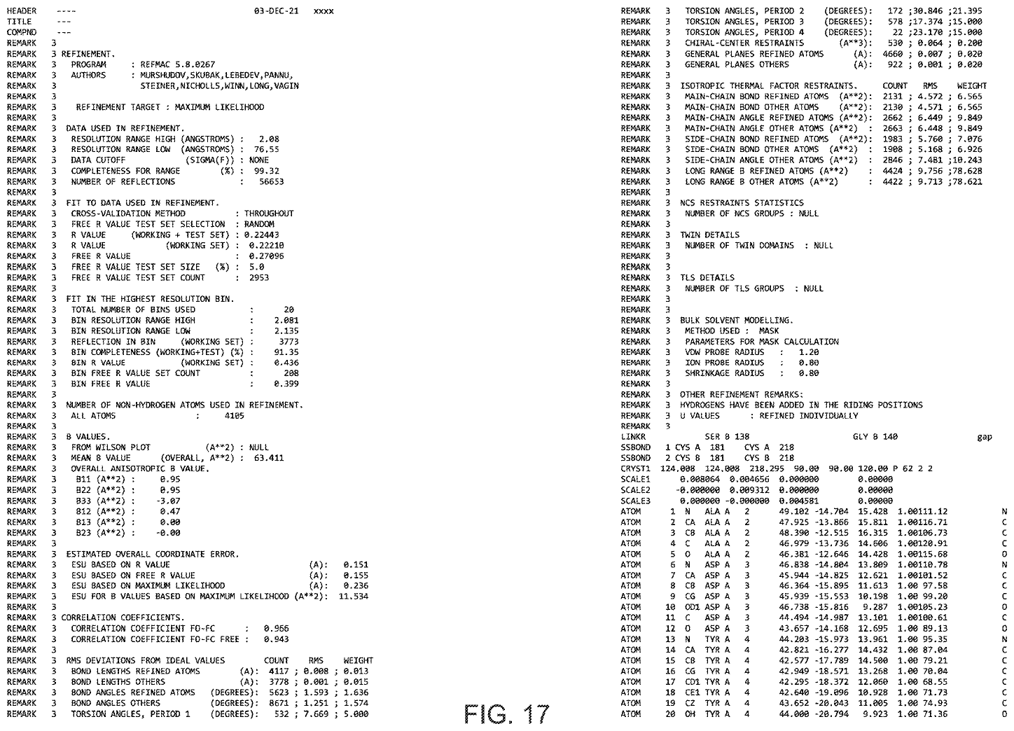

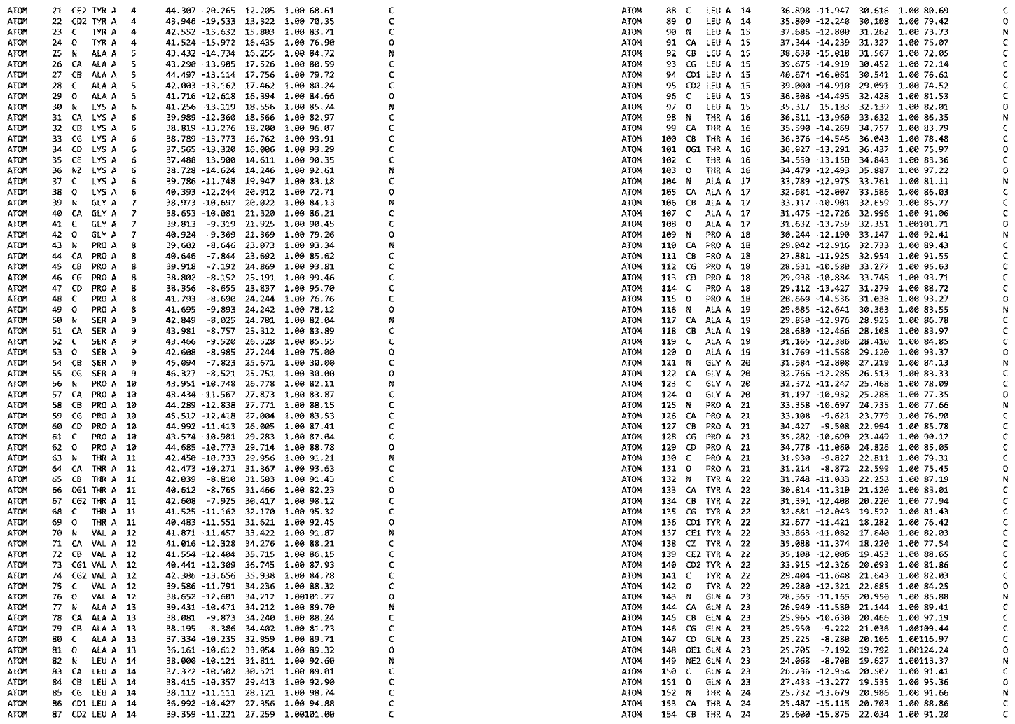

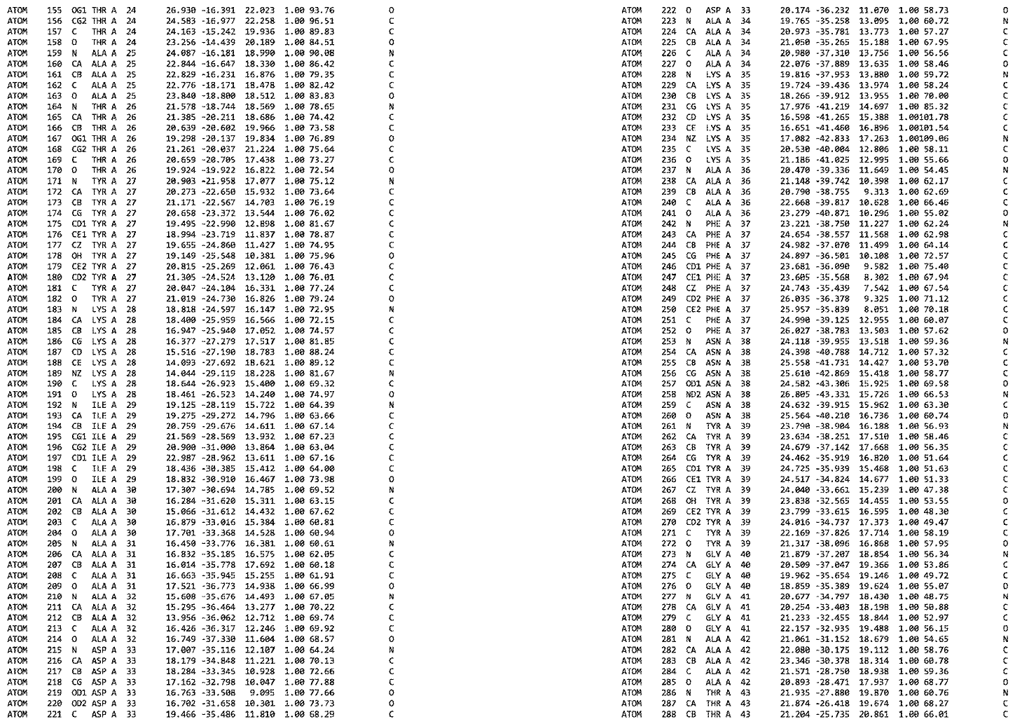

The structure of CI5En2-A9S is shown in FIG. 10. The atomic co-ordinates are listed in FIG. 17.

The project leading to this application has received funding from the European Research Council (ERC) under the European Union's Horizon 2020 research and innovation programme under grant agreements No 695669 and 685474.

### Example 10: Manufacture of Lignin Nanoparticles

The lignin nanoparticles were prepared following a precipitation and solvent evaporation technique as previously described for PET.¹ Briefly, 250 mg lignin powder was dissolved in 1,1,3,3,3,-hexafluoro-2-propanol (5 mL) for at least one hour. This solution was added dropwise (1 mL/min) to double distilled H₂O (50 mL, cooled in an ice bath) until an accumulation of aggregated particles on the water surface was observed. At the same time the water was rigorously stirred using an Ultra Turrax® stirrer at 8000 rpm (IKA, Germany). Then the suspension was filtered using Whatman filter paper (8 μm diameter). Afterwards the remaining solvent was evaporated. No surfactant was needed to prevent sedimentation.

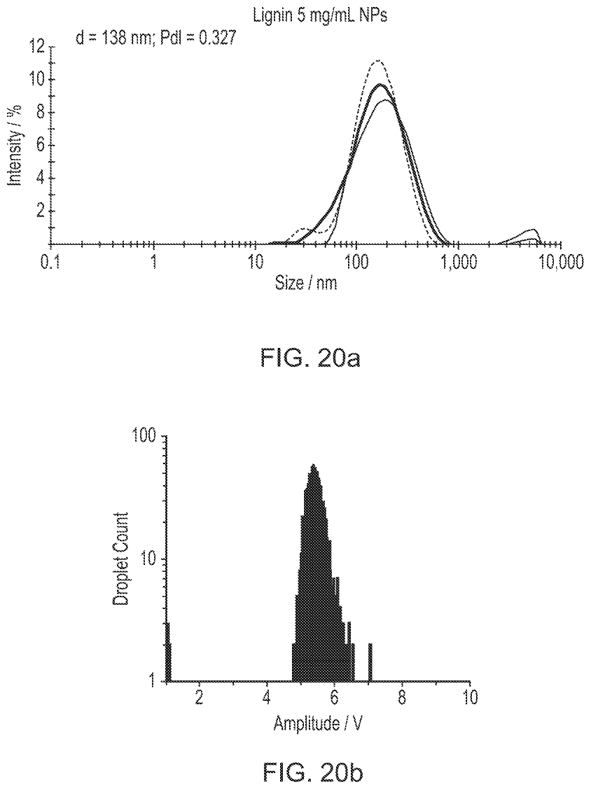

RESULTS: The results are shown in FIG. 20. A) The particle size distribution was evaluated by measuring the hydrodynamic diameter by dynamic light scattering on a Zetasizer NanoZS (Malvern, UK). DLS measurement suggested a narrow size distribution of lignin nanoparticles around 138 nm and monodispersity (Pdl: 0.3). B) When using the nanoparticles, no sedimentation was observed in the microfluidic droplets, even in droplet measurements taken after several days. No surfactant was needed to obtain stabilisation.

### Example 11: Novel Enzymes for nylon Depolymerization

Sequence space exploration was used for the initial discovery of novel nylonases. Homologues of NylC (NCBI access code: Q1EPR5.2) were extracted from MGnify database and used to generate a sequence network composed of 1583 nodes and 69291edges sequences. The first connections of MGYP000645618208 (sequence retrieved from MGnify under the listed code) were identified and the sequence space divided into clusters (from 1 to 3). Potential nylonase candidates were selected from each cluster, including: Ny15 (MGnify code: MGYP000544655629, from cluster 1) and Nyl9 (MGnify code: MGYP000403660814, from cluster 3).

### Enzyme Assays for nylon Nanoparticles

nylon nanoparticles (approximately 0.3 mg of dried powder) were incubated with 10 μM purified and TEV cleaved proteins in Tris-HCl and 100 mM NaCl buffer (pH 8) at 37° C. The reactions were terminated at different time points (t0, t3d and t7d) by heat denaturation. The supernatant obtained by centrifugation (16,100×g, 10 min) was analysed by HPLC (1260 Infinity II, Agilent; Nucleodur C18 analytical EC standard column (5 μm, 4×125 mm, Macherey-Nagel, Germany) to detect the monomer 6-aminohexanoic acid (Ahx). As the mobile phase buffer A (distilled water) and buffer B (acetonitrile) were used at a flow rate of 1 mL/min. Buffer A was kept at 100% at the beginning. After 5 min the mobile phase was changed gradually to 100% B until 10 min and then back to 100% A until 15 min. Ahx was detected at 220 nm by comparison with authentic samples and quantified by comparison to calibration curves.

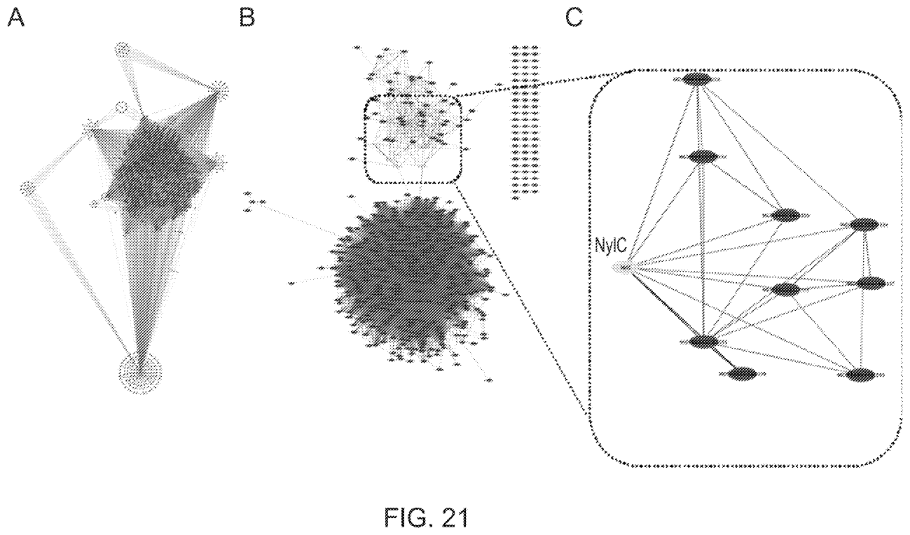

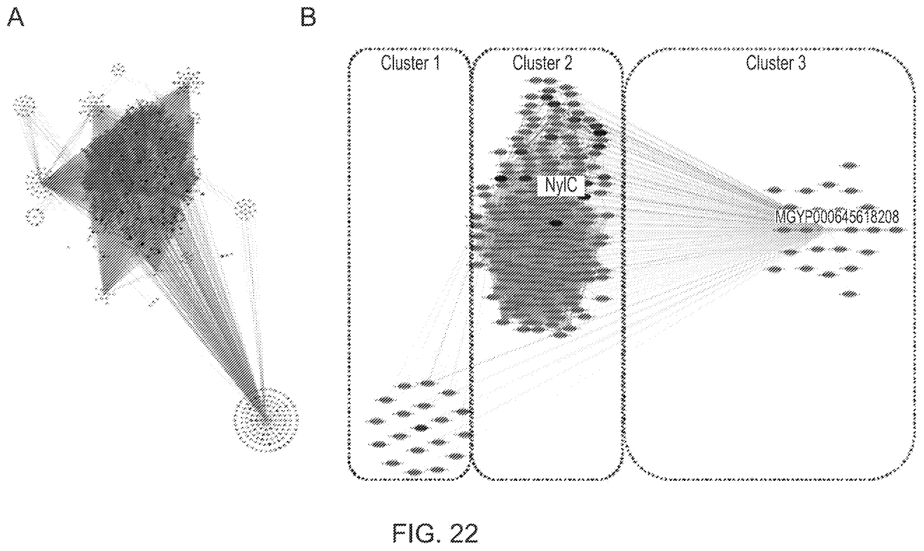

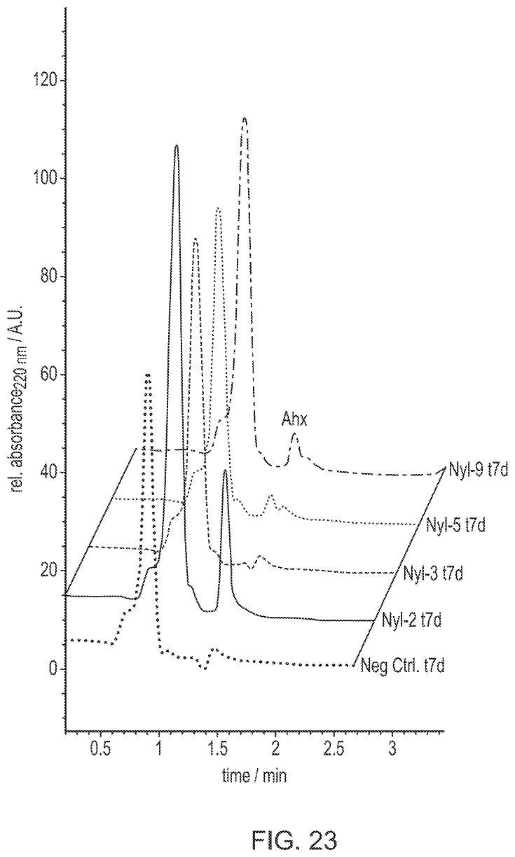

RESULTS: The results are shown in FIGS. 21-28.

The following new nylonases were found:

| Name | DNA SEQ ID NO. | Amino acid SEQ ID NO. |
|---|---|---|
| NYL2 | 7 | 8 |
| NYL5 | 9 | 10 |
| NYL9 | 11 | 12 |
| NYL10 | 13 | 14 |
| NYL11 | 15 | 16 |
| NYL12 | 17 | 18 |
| NYL14 | 19 | 20 |
| NYL18FL | 21 | 22 |
| NYL20FL | 23 | 24 |
| NYL24FL | 25 | 26 |
| NYL25FL | 27 | 28 |
| SOIL1 | 29 | 30 |
| MARINE2 | 31 | 32 |

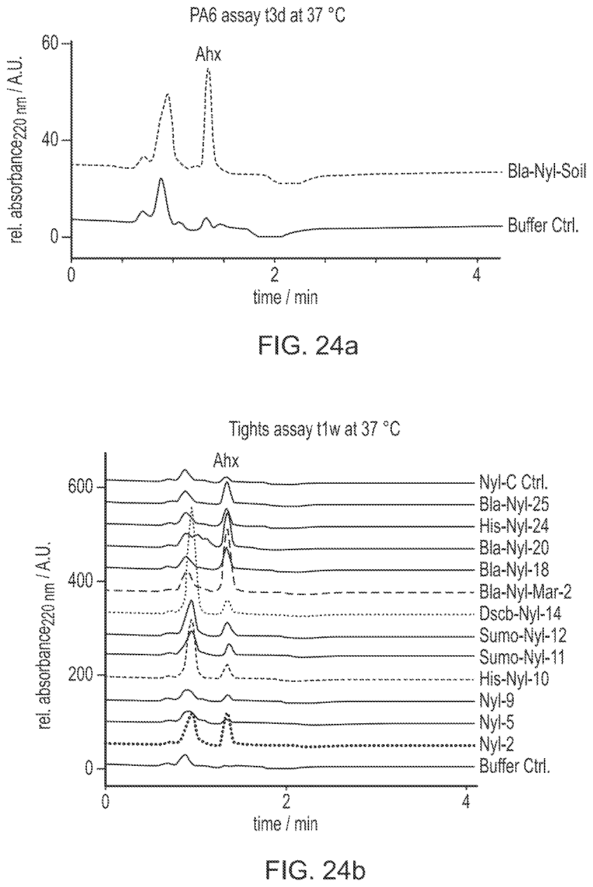

FIG. 24 shows various of the above enzymes tested on either PA6 nanoparticles (NPs) (a) or post-consumer tights samples containing 85% nylon (b). HPLC measurements confirm the generation of the nylon monomer 6-aminohexanoic acid (Ahx).

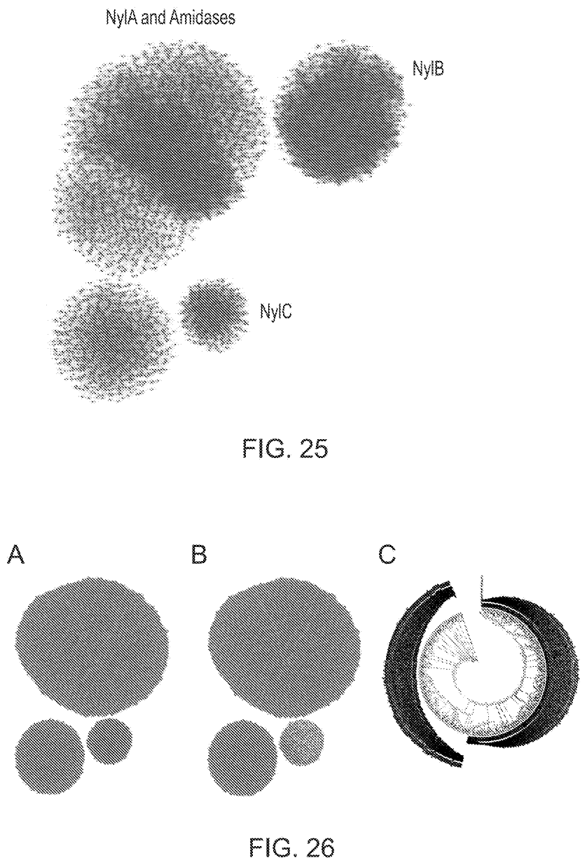

FIG. 25 shows the discovery of some of the new nylonases listed in the table above. In particular, among the new nylonases are Nyl-10 (Uniprot code A0A1C6V3K0), Nyl-11 (Uniprot code A0A138ZXG5), Nyl-12 (Uniprot code A0A1M7HAK8) and Nyl-14 (Uniprot code WP243569406.1). Nyl-10, Nyl-11, Nyl-12 and Nyl14 are far in the sequence space from known nylonases, sharing 49%, 33%, 30% and 56% sequence identity with NylC, respectively.

FIG. 26 shows the discovery of some of the other new nylonases above. In particular, among the new nylonases are Nyl 18-FL (MGnify code MGYP000370831902/2-317), 20-FL (MGnify code MGYP000123679597/3-313), 24-FL (MGnify code MGYP000614730541/11-353) and 25-FL (MGYP000554001943/18-373), sharing 38%, 41%, 30% and 33% sequence identity with NylC, respectively.

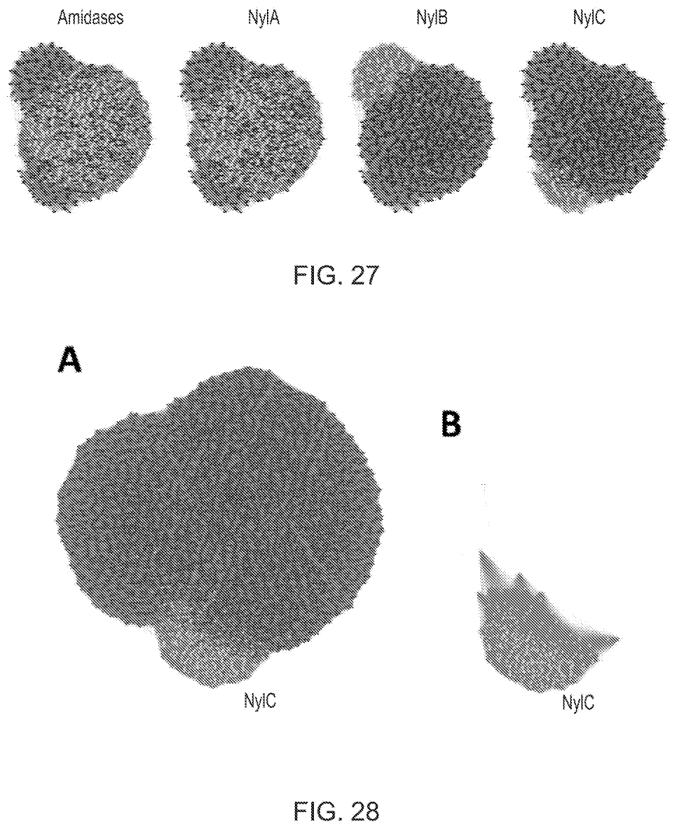

FIG. 27 shows the discovery of Soil1, a new nylonase described at the list above. Soil 1 (MGnify code MGYP000120183263/1-286) shares 20% sequence identity with NylC.

FIG. 28 shows the discovery of Marine2, a new nylonase described at the list above. Marine 2 (MGnify code MGYP000645432643/3-305) shares 19% sequence identity with NylC.

1. A method for screening for an optimised plastic degrading enzyme, the method comprising:
a) encapsulating a gene library with variant gene sequences of the plastic degrading enzyme into a plurality of microfluidic droplets, each microfluidic droplet comprising at least one variant gene sequence of the plastic degrading enzyme and each microfluidic droplet additionally comprising:
i) an expression system for expressing the gene; and
ii) a plastic particle;
b) detecting degradation of the plastic particle in the microfluidic droplet; and
c) selecting microfluidic droplets where plastic degradation has been detected.

2. A method for identifying a plastic degrading enzyme, the method comprising:
a) encapsulating a gene library into a plurality of microfluidic droplets, each microfluidic droplet comprising at least one gene and each microfluidic droplet additionally comprising:
i) an expression system for expressing the gene; and
ii) a plastic particle;
b) detecting degradation of the plastic particle in the microfluidic droplet; and
c) selecting microfluidic droplets where plastic degradation has been detected.

3. The method of claim 1, wherein the gene library is a directed evolution library.

4. The method of claim 1, wherein detecting degradation is by detecting a reduction in light scattering by the plastic particle.

5. The method of claim 1, wherein the plastic particle comprises a polyester, polyamide, polyurethane or polyolefin, optionally wherein the plastic particle comprises any one or more of the following: polyethylene terephthalate (PET), polytrimethylene terephthalate (PTT), polybutylene terephthalate (PBT), polyethylene isosorbide terephthalate (PEIT), polylactic acid (PLA), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly (ethylene adipate) (PEA), polyethylene naphthalate (PEN), polyethylene (PE), polystyrene (PS), polypropylene (PP), polyvinyl chloride (PVC), polyurethane (PUR), polyamides (PA), polycarbonate (PC).

6. The method of claim 1, wherein the method is preceded by a screening method to identify the gene sequence of a plastic degrading enzyme, wherein the screening method comprises:
a) encapsulating a gene library into a plurality of microfluidic droplets, each microfluidic droplet comprising at least one gene and each microfluidic droplet additionally comprising:
i) an expression system for expressing the gene; and
ii) a soluble plastic mimic;
b) detecting cleavage of the soluble plastic mimic in the microfluidic droplet; and
c) selecting microfluidic droplets where cleavage has been detected, optionally wherein the gene library is a metagenomic library.

7-17. (canceled)

18. The method of claim 2, wherein the gene library is a metagenomic library.

19. The method of claim 2, wherein the method further comprises making a gene library with variant gene sequences of the identified plastic degrading enzyme and performing a method for screening for an optimised plastic degrading enzyme, the method comprising:
a) encapsulating a gene library with variant gene sequences of the plastic degrading enzyme into a plurality of microfluidic droplets, each microfluidic droplet comprising at least one variant gene sequence of the plastic degrading enzyme and each microfluidic droplet additionally comprising:
i) an expression system for expressing the gene; and
ii) a plastic particle;
b) detecting degradation of the plastic particle in the microfluidic droplet; and
c) selecting microfluidic droplets where plastic degradation has been detected.

20. The method of claim 2, wherein detecting degradation is by detecting a reduction in light scattering by the plastic particle.

21. The method of claim 2, wherein the plastic particle comprises a polyester, polyamide, polyurethane or polyolefin, optionally wherein the plastic particle comprises any one or more of the following: polyethylene terephthalate (PET), polytrimethylene terephthalate (PTT), polybutylene terephthalate (PBT), polyethylene isosorbide terephthalate (PEIT), polylactic acid (PLA), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly (ethylene adipate) (PEA), polyethylene naphthalate (PEN), polyethylene (PE), polystyrene (PS), polypropylene (PP), polyvinyl chloride (PVC), polyurethane (PUR), polyamides (PA), polycarbonate (PC).

22. The method of claim 2, wherein the method is preceded by a screening method to identify the gene sequence of a plastic degrading enzyme, wherein the screening method comprises:
a) encapsulating a gene library into a plurality of microfluidic droplets, each microfluidic droplet comprising at least one gene and each microfluidic droplet additionally comprising:
i) an expression system for expressing the gene; and
ii) a soluble plastic mimic;
b) detecting cleavage of the soluble plastic mimic in the microfluidic droplet; and
c) selecting microfluidic droplets where cleavage has been detected, optionally wherein the gene library is a metagenomic library.
